(19) United States
(12) Patent Application Publication
(10) Pub. No.: US 2024/0366736 A1
(43) Pub. Date: Nov. 7, 2024

(54) METHOD FOR MANAGING PAIN

(71) Applicant: University of Miami, Miami, FL (US)

(72) Inventors: Roy Levitt, Miami, FL (US); Gerald Z. Zhuang, Miami, FL (US)

(21) Appl. No.: 18/418,736

(22) Filed: Jan. 22, 2024

Related U.S. Application Data
(62) Division of application No. 16/629,477, filed on Jan. 8, 2020, now Pat. No. 11,911,450.
(60) Provisional application No. 62/532,182, filed on Jul. 13, 2017.

(51) Int. Cl. A61K 38/51; A61K 9/00; A61P 25/02; A61P 29/00; C12N 9/88; C12N 15/86

(52) U.S. Cl. CPC A61K 38/51; A61K 9/0019; A61K 9/0085; A61P 25/02; A61P 29/00; C12N 9/88; C12N 15/86; C12Y 402/01001; C12N 2750/14143

(57) ABSTRACT

The invention provides a method of treating or preventing pain in a subject in need thereof. The method comprising administering to the subject an expression vector comprising a nucleic acid sequence encoding carbonic anhydrase (10) or carbonic anhydrase (11) such that the nucleic acid is expressed to produce carbonic anhydrase (10) or carbonic anhydrase (11). Alternatively, the method comprising administering to the subject an expression vector comprising a nucleic acid sequence encoding a carbonic anhydrase (8) fragment such that the nucleic acid is expressed to produce the carbonic anhydrase (8) fragment.

### GRANT FUNDING DISCLOSURE

This invention was made with government support under grant number DE022903, awarded by the National Institutes of Health. The government has certain rights in the invention.

### FIELD OF DISCLOSURE

The invention relates to materials and methods for treating or preventing pain.

### INCORPORATION BY REFERENCE OF MATERIALS SUBMITTED ELECTRONICALLY

This application contains, as a separate part of the disclosure, a Sequence Listing in computer readable form (Filename: 51774A_Seqlisting.txt; Size: 73,7002 bytes; Created: Jul. 13, 2018), which is incorporated by reference in its entirety.

### BACKGROUND

Persistent pain costs about $650 billion annually in health care costs and lost productivity. A National Health Interview Survey conducted in 2012 estimated that nearly 50 million American adults suffer from significant chronic pain or severe pain. Press release, American Pain Society, Aug. 18, 2015, found at americanpainsociety.org/about-us/press-room/nih-study-shows-prevalence-of-chronic-or-severe-pain-in-u-s-adults. Other studies suggest the burden of pain is higher.

Currently available treatments for chronic pain are associated with disadvantages that leave most patients inadequately treated. Current local anesthetics, for example, are short acting and disabling due to complete sensory and motor blockade. Opioid drugs, including morphine, are the primary treatment for, e.g., post-operative and chronic pain conditions. Long-term opioid use in treating chronic, non-cancer pain has increased dramatically over the past few decades, and opioid abuse, tolerance and dependence are major public health concerns. Indeed, side-effects of opioid administration (e.g., tolerance, drug dependence/addiction, respiration depression, constipation, nausea, pruritis, sedation, and mood swings), limit opioids' therapeutic potential, and the absence of suitable alternatives has led to an epidemic of opioid overuse, abuse, and life-threatening complications. In the United States, prescription opioid abuse costs alone were estimated at about $55.7 billion in 2007. Almost half this cost was attributed to workplace costs (e.g., lost productivity), 45% to healthcare costs (e.g., abuse treatment), and 9% to criminal justice costs. Birnbaum et al., Pain medicine 2011;12:657-67.

Despite a considerable amount of research into pain medications, there remains a need for therapeutic options that provide analgesia while minimizing (or avoiding) the adverse effects associated with opioid use.

### SUMMARY

The disclosure provides a method of treating or preventing pain in a subject (e.g., human) in need thereof. In one aspect, the method comprises administering to the subject an expression vector (e.g., a viral vector, such as an adeno-associated viral vector or a herpes simplex viral vector) comprising a nucleic acid sequence encoding carbonic anhydrase 10 or carbonic anhydrase 11 such that the nucleic acid is expressed to produce carbonic anhydrase 10 or carbonic anhydrase 11. Alternatively, the expression vector (e.g., a viral vector, such as an adeno-associated viral vector or a herpes simplex viral vector) comprises a nucleic acid sequence encoding carboxyl anhydrase 8 or a carbonic anhydrase 8 fragment. Human and mouse versions of carbonic anhydrases are contemplated (CA8/Car8, CA8/Car8 fragments, CA10/Car10 and/or CA11/Car11). It will be appreciated that descriptions herein relating to human versions of carbonic anhydrases (e.g., CA8, CA8 fragments, CA10 and/or CA11) also apply to the mouse versions (Car8, Car8 fragments, Car10, and/or Car11). The nucleic acid sequence encoding the fragment of carbonic anhydrase 8 (CA8 human or Car8 mouse) comprises (or consists essentially of or consists of) the first three exons of the carbonic anhydrase 8 coding sequence. Optionally, the nucleic acid sequence encoding the fragment of carbonic anhydrase 8 is alternative transcript CA8-204, comprising the first three exons with an elongated exon 3 and retained intron. In various aspects, the carbonic anhydrase is an antagonist of ITPR1-activation (pITPR1) and ITPR1-mediated intracellular calcium release.

In various aspects, the pain is neuropathic pain, cancer pain, or inflammatory pain. In various aspects, the method comprises administering the expression vector to the dorsal root ganglion of the subject or administering the expression vector via intraarticular injection, intradermal delivery, or intra-oral delivery.

The foregoing summary is not intended to define every aspect of the invention, and additional aspects are described in other sections, such as the Detailed Description. The entire document is intended to be related as a unified disclosure, and it should be understood that all combinations of features described herein are contemplated, even if the combination of features are not found together in the same sentence, or paragraph, or section of this document. In addition, the invention includes, as an additional aspect, all embodiments of the invention narrower in scope in any way than the variations specifically mentioned above. With respect to aspects of the invention described or claimed with “a” or “an,” it should be understood that these terms mean “one or more” unless context unambiguously requires a more restricted meaning. With respect to elements described as one or more within a set, it should be understood that all combinations within the set are contemplated. If aspects of the invention are described as “comprising” a feature, embodiments also are contemplated “consisting of” or “consisting essentially of” the feature.

Although the applicant(s) invented the full scope of the claims appended hereto, the claims appended hereto are not intended to encompass within their scope the prior art work of others. Therefore, in the event that statutory prior art within the scope of a claim is brought to the attention of the applicants by a Patent Office or other entity or individual, the applicant(s) reserve the right to exercise amendment rights under applicable patent laws to redefine the subject matter of such a claim to specifically exclude such statutory prior art or obvious variations of statutory prior art from the scope of such a claim. Variations of the invention defined by such amended claims also are intended as aspects of the invention. Additional features and variations of the invention will be apparent to those skilled in the art from the entirety of this application, and all such features are intended as aspects of the invention.

### BRIEF DESCRIPTION OF THE FIGURES

FIGS. 1A-1C are bar graphs illustrating relative pITPR1 density (FIG. 1A), relative V5 density (FIG. 1B), and relative pITPR1 density (FIG. 1C). Overexpression of Car10/CA10 in HEK293 cells inhibits forskolin-induced ITPR1 phosphorylation (pITPR1). HEK293 cells were transfected with AAV-V5 vectors expressing murine (Car10) and human (CA10). Western blot analysis demonstrates that forskolin increases pITPR1 levels in a dose-dependent manner (FIG. 1A). Following V5-Car10 and V5-CA10 vector transfection, CA10 and Car10 protein overexpression was demonstrated using V5 tag on western blotting (FIG. 1B). Car10 and CA10 overexpression reduced ITPR1 phosphorylation in response to 1 micromolar forskolin stimulation in HEK293 cells (FIG. 1C). N=6, data are presented as means ±SEM ** P<0.01, *** P<0.001, one-way ANOVA.

FIG. 2 is a bar graph illustrating that overexpression of AAV-V5-Car10 and AAV-V5-CA10 in HEK293 cells inhibited 1 μM ATP-induced cytoplasmic calcium release (as indicated by Fura-2 (340/380 ratio) (y-axis). Calcium imaging data demonstrated that car10 and CA10 protein overexpression inhibited 1 μM ATP-induced cytoplasmic calcium release in HEK293 cells when compared to empty vector control. Car10 and CA10 overexpression significantly inhibited free calcium release (P<0.001). N=4 coverslips and a total of 200 cells per sample ** P<0.01, *** P<0.001, by two way ANOVA followed by Bonferroni test.

FIG. 3 is a bar graph illustrating paw withdrawal latency (seconds) (y-axis) on various days of study (x-axis), demonstrating that gene transfer of V5-Car10 and V5-CA10 results in thermal anti-hyperalgesia in a carrageenan inflammatory pain mouse model. Thermal responses following overexpression of V5-Car10 and V5-CA10 via sciatic nerve injections of saline, AAV8-null, AAV8-V5-Car10 and AAV8-V5-CA10 viral particles (1.06E¹⁴ viral particles and 1.66E¹⁴ viral particles, respectively) in C57BL/6J mice. Basal thermal latencies increased by day 15 after injection of AAV8-V5-Car10 and AAV8-V5-CA10 viral particles but not after saline or viral particles containing empty vector. Following carrageenan injections at the end of day 16, the saline and AAV8-null containing viral particles showed a significant reduction from baseline thermal latencies. However, AAV8-V5-Car10 and AAV8-V5-CA10 injected groups did not differ from baseline (N=8. * P<0.05, ** P<0.01, *** P<0.001, by two way ANOVA followed by Bonferroni Post-hoc test).

FIG. 4 is a bar graph illustrating paw withdrawal latency (seconds) (y-axis) on various days of study (x-axis), demonstrating that gene transfer of V5-Car10 and V5-CA10 results in thermal anti-hyperalgesia in a Complete Freund's adjuvant (CFA) chronic inflammatory pain mouse model. Thermal responses are shown following sciatic nerve injections of saline, AAV8-null, AAV8-V5-Car10 and AAV8-V5-CA10 viral particles (1.06E¹⁴ viral particles and 1.66E¹⁴ viral particles, respectively). Basal thermal latencies increased by day 15 after injection of AAV8-V5-Car10 and AAV8-V5-CA10 viral particles, but not after saline or viral particles containing empty vector. Following CFA injections at the end of day 16 (after thermal latencies were measured), all groups had a significant reduction from baseline. Starting from Day 24 the AAV8-V5-Car10 and AAV8-V5-CA10 injected groups showed analgesia (thermal latencies above baseline, similar to Days 15 and 16 after viral injections), while the control groups were unchanged from prior to CFA injection. (N=8. * P<0.05, ** P<0.01, *** P<0.001, by two way ANOVA followed by Bonferroni Post-hoc test).

FIG. 5 is a bar graph illustrating paw withdrawal latency (seconds) (y-axis) on various days of study (x-axis), demonstrating that gene transfer of V5-Car10 and V5-CA10 prevents mechanical hyperalgesia in a neuropathic (Chung) mouse pain model. Mechanical withdrawal thresholds are shown following sciatic nerve injections of saline, AAV8-null, AAV8-V5-Car10 and AAV8-V5-CA10 viral particles (1.06E¹⁴ viral particles and 1.66E¹⁴ viral particles, respectively) in C57BL/6J mice. AAV8-V5-Car10 increased mechanical withdrawal thresholds above baseline (analgesia) on Day 12 after viral injections, and this was maintained through Day 22 despite spinal nerve ligation on Day 19. There was no similar increase in withdrawal thresholds in the other groups. (N=8. * p<0.05, ** p<0.01, *** p<0.001, by two way ANOVA followed by Bonferroni test).

FIG. 6 is a bar graph illustrating the relationship between paw withdrawal latency (seconds) (y-axis) at approximately 30 minutes after the morphine dose was given (intraperitoneal dosing) (x-axis). Elevations were significant when compared with saline at 10, 30, and 60 mg/kg doses.

FIG. 7 is a graph illustrating the relationship between paw withdrawal latency (seconds) (x-axis), to morphine equivalents oral dose in a 60 kg human (intraperitoneal dosing assessed at approximately 30 minutes after dosing; data are extrapolated to human equivalent dosing using allometric conversion) (y-axis). The elevations in the paw withdrawal latency after transfer of V5-Car10 and V5-CA10 produces profound analgesia in these mouse models as described in FIGS. 2 through 5 when compared to the human oral equivalent dose.

FIG. 8 is an amino acid sequence encoding CA10 (SEQ ID NO: 9).

FIG. 9 is a nucleotide sequence encoding CA10-203 (SEQ ID NO: 12).

FIG. 10 is a nucleotide sequence encoding a CA8 fragment, CA8-204 (SEQ ID NO: 13).

FIG. 11 is a bar graph illustrating paw withdrawal latency (seconds) (y-axis) on various days of study (x-axis), demonstrating the analgesic effect of expression of V5-CA8WT (left bar=post-SNL CA8WT; right bar=post-SNL CA8WT) and V5-CA8MT (CA8MT represents the S100P point mutation that destabilizes the protein and leads to rapid degradation by the proteasome; Turkmen S, et al., PLOS Genet 5, e1000487) (center bar=post-SNL CA8MT) in a neuropathic (Chung) mouse pain model. Mechanical withdrawal thresholds are shown following sciatic nerve injections of AAV viral particles (approximately 1.0 X 1014 viral particles) in C57BL/6J mice. AAV8-V5-CA8WT increased mechanical withdrawal thresholds above baseline (analgesia) by Day 7 after administration, and this was maintained through Day 38 despite spinal nerve ligation on Day 3. There was no similar increase in withdrawal thresholds after administration of an expression vector encoding of AAV8-V5-CA8MT. (N=8. * p<0.05, ** p<0.01, *** p<0.001, by two way ANOVA followed by Bonferroni test). The results of the study are surprising. First, it was unclear whether CA8 would function against the mouse ITPR1 target. These data show human proteins function in mouse cells/tissue; and that in vitro bioassays described herein can be used to test pharmacodynamics of CA analgesic peptides and variants. Additionally, neuropathic pain is believed to arise from different mechanisms than inflammatory pain. Moreover, medications that work well for inflammatory pain do not produce anti-hyperalgesia or anti-allodynia in neuropathic pain models. Surprisingly CA8 is effective in preventing and treating neuropathic pain.

FIG. 12A is a bar graph illustrating gene expression for CA8 fragments encoded by the first three exons of the CA8 coding sequence: an alternative splice fragment “CA ALT G” (“G” allele at SNP rs6471859) and wildtype CA8 “CA ALT C” (C allele for SNP rs6471859) as measured by qPCR in NBL cells. The wildtype CA8 splices normally, and NBL cells produce this fragment encoded by the first three exons of CA8, almost exclusively. Expression of the “G” allele at SNP rs6471859 leads to alternative splicing, and there is almost no detectable CA8-204 product with an extended exon three with a retained intron in NBL cells. Thus, essentially all CA8 transcript and protein fragment encoded by the first three exons of CA8 is produced and stable in NBL cells. FIG. 12B is a bar graph demonstrating that this CA8 fragment (encoded by the first three exons only) inhibits ATP-stimulated calcium release in NBL cells. The wildtype CA8 (C allele for SNP rs6471859) splices normally, and this fragment of the wildtype gene product is produced in NBL cells. Expression of the “G” allele at SNP rs6471859 leads to alternative splicing, and production of CA8-204 protein is minimal in NBL cells. This fragment mediates no inhibition in these cells as compared to the vectors expressing CA8-201 (wildtype protein, left bar) or the truncated CA8 fragment produced by the “C” allele (right bar).

FIG. 13A is a bar graph illustrating expression of CA8 fragments in HEK293 cells as measured by qPCR. Expression of the “G” allele at SNP rs6471859 leads to alternative splicing in these cells, producing the CA8-204 product with an extended exon 3 with a retained intron in HEK293 cells (left bar). There is essentially no detectable wildtype CA8 (C allele for SNP rs6471859) in HEK293 cells (right bar), which produces a fragment corresponding to the first three exons of CA8. Thus, essentially all vector expression in HEK293 cells is the CA8-204 alternative transcript. FIG. 13B is a bar graph demonstrating that CA8-204 inhibits ATP-stimulated calcium release in HEK293 cells. The vector expressing the “G” allele at SNP rs6471859 leads to alternative splicing and production of CA8-204 protein found in HEK293 cells, which inhibits ATP-induced calcium release compared to the vectors expressing wildtype CA8 (CA8-201) and the fragment coded for by the “C” allele.

FIG. 14 is a diagram of an exemplary HSV expression vector for CA8 delivery. TrkAp-CA8-Flag, Nav1.7p-CA8-204-Flag, Nav1.8p-CA8-204-Flag, and (not shown) Tet-Advillin-CA8-204-Flag) cassettes may be incorporated into the ICP4 locus. The base vector is optionally deleted for ICP0, ICP4 IE regulatory genes, Joint region, ICP27, ICP47, ICP22 IE genes “TAATGARAT”, and the UL41 vhs gene. A gB: N/T mutation may be present (which in various embodiments enhances cell entry), and BAC sequences are located between loxP sites in intergenic regions. The vector construct is also suitable for delivery of CA8 fragments.

FIG. 15 is a diagram of an exemplary HSV expression vector for CA8 delivery. TrkAp-CA8-Flag, Nav1.7p-CA8-Flag, Nav1.8p-CA8-Flag, and (not shown) Tet-Advillin-CA8-Flag cassettes may be incorporated into the ICP4 locus. The base vector is deleted for ICP0, ICP4 IE regulatory genes, Joint region, ICP27, ICP47, ICP22 IE genes “TAATGARAT”; and UL41 vhs gene. A gB: N/T mutation enhances cell entry, and BAC sequences are located between loxP sites in intergenic regions.

FIG. 16 is a diagram of an exemplary HSV expression vector for CA10 Biotherapeutic delivery. Diagram of TrkAp-CA10-Flag, Nav1.7p-CA10-Flag, Nav1.8p-CA10-Flag, and (not shown) Tet-Advillin-CA10-Flag cassettes may be incorporated into the ICP4 locus. The base vector is deleted for ICP0, ICP4 IE regulatory genes, Joint region, ICP27, ICP47, ICP22 IE genes “TAATGARAT”; and UL41 vhs gene. A gB: N/T mutation enhances cell entry, and BAC sequences are located between loxP sites in intergenic regions.

FIG. 17 is a diagram of an exemplary HSV expression vector for CA11 delivery. TrkAp-CA11-Flag, Nav1.7p-CA11-Flag, Nav1.8p-CA11-Flag, and (not shown) Tet-Advillin-CA11-Flag cassettes that represent inducible promoter systems may also be incorporated into the ICP4 locus. The base vector is deleted for ICP0, ICP4 IE regulatory genes, Joint region, ICP27, ICP47, ICP22 IE genes “TAATGARAT”; and UL41 vhs gene. A gB: N/T mutation enhances cell entry, and BAC sequences are located between loxP sites in intergenic regions.

FIG. 18 is a bar graph illustrating NGF-induced intracellular calcium release (y-axis) in NBL cells in response to various doses of NGF (1, 10, 100 ng/mL) (x-axis). HEK293 cells also respond to NGF with increased intracellular calcium release (data not shown).

FIG. 19 is a bar graph illustrating that NGF-induced intracellular calcium release (y-axis) in NBL cells is nearly completely by overexpression of wildtype CA8, but not overexpression of a mutant form of CA8. ATP induced intracellular calcium release is also inhibited by CA10 overexpression [data not shown].

FIG. 20 is a bar graph illustrating blockage of NGF-induced intracellular calcium release by the selective ITPR1 inhibitor 2-APB in NBL cells. Thus, NGF signaling in NBL cells is almost exclusively through ITPR1 and nearly completely inhibited by this ITPR1 selective inhibitor. Similar inhibition by 2-APB was also observed in HEK293 cells [data not shown].

FIG. 21A is a schematic of the 5HT-3 receptor promoter and comparison of mouse (SEQ ID NO: 14) and human sequences (SEQ ID NO: 15) (Journal of Neuroscience 15, Aug. 1998, 18 (16) 6186-6194).

FIG. 21B provides the 5HT-3 receptor promoter sequence (SEQ ID NO: 16).

FIG. 22 provides the sequence of NPY-Y1 Receptor Promoter-exon 1A (SEQ ID NO: 17) (JBC Vol. 270, No. 45, Issue of November 10, pp. 27272-27276, 1995).

FIG. 23 provides the sequence of NPY-Y1 Receptor Promoter-exon 1B (SEQ ID NO: 18) (JBC Vol. 270, No. 45, Issue of November 10, pp. 27272-27276, 1995).

FIG. 24 provides the sequence of NPY-Y1 Receptor Promoter-exon IC (SEQ ID NO: 19) (JBC Vol. 270, No. 45, Issue of November 10, pp. 27272-27276, 1995).

FIG. 25A provides the sequence of mouse (SEQ ID NO: 20), rat (SEQ ID NO: 21), and human (SEQ ID NO: 22) Nav1.8 Promoter SCN10A (J Neurochem. 2008 August; 106 (3): 1209-1224).

FIG. 25B provides the sequence of human Nav1.8 Promoter SCN10A (SEQ ID NO: 23).

FIG. 26 provides the sequence of Nav1.7 Promoter SCN9A (SEQ ID NO: 24).

FIG. 27A provides the sequence of human Trk-A promoter (SEQ ID NO: 25) (J. Biol. Chem. 1998, 273:39-44). Potential DNA binding protein binding sites are marked by boxes.

FIG. 27B provides the sequence of Trk-A promoter (SEQ ID NO: 26).

FIG. 28 provides the sequence of an Advillin promoter (SEQ ID NO: 27).

FIG. 29 provides the sequence of CGRP Receptor promoter (SEQ ID NO: 28).

FIG. 30 provides the sequence of GRIN3A promoter (SEQ ID NO: 29).

FIG. 31 is a graph showing the gene transfer of CA fragment CA8^{204C} in an inflammatory pain animal model produced analgesia anti-hyperplasia.

FIG. 32 is a graph showing the gene transfer of CA fragment CA8^{204G} in an inflammatory pain animal model produced analgesia anti-hyperplasia.

FIG. 33 depicts a gel showing that RYR1 binds to CA10 (Car10) as demonstrated by immunoprecipitation (IP) and western blotting (WB) detection.

FIG. 34A and 34B are gels and provide bar graphs showing that Car10 and CA10 inhibited forskolin-induced pITPR1. FIG. 34A provides results for untreated controls (no forskolin). FIG. 34B provides results from treatment with 1 μM forskolin.

FIG. 35 is a bar graph showing that expression of V5-Car10 and V5-CA10 in HEK293 cells inhibits ATP-induced cytoplasmic calcium release.

FIG. 36 is a bar graph showing that 5HT-induced RYR-dependent calcium release in NBL cells is inhibited by ryanodine.

FIG. 37 is a bar graph showing that overexpression of V5-Car10 and V5-CA10 in NBL cells inhibits 50 μM 5HT-induced cytoplasmic calcium release.

FIG. 38 shows construction of pCMV-N-flag-CA8-204^{G} and pCMV N-flag-CA8-204^{C}.

FIG. 39 shows construction of pAAV-flag-CA8-204^{G} (ALT G) and pAAV-flag-CA8-204^{C} (ALT C).

FIG. 40 shows that CA8-204^{G} inhibition of calcium release (Ca²⁺ Fura2 imaging) in NBL cells.

FIG. 41 show that differential tissue expression of CA8 ALT (G) (CA8-204G and CA8 ALT (C) (CA8-204C) in HEK293 and NBL cells.

FIG. 42A and FIG. 42B are graphs showing that CA8-204^{C} (FIG. 42B) and CA8-204^{G} (FIG. 42A) fragments have variable tissue expression.

FIG. 43 shows that CA8-204^{C} fragment inhibits ATP stimulated calcium release in NBL cells.

FIG. 44 is a gel showing that CA8-204^{G} or CA8-204^{C} peptide fragments are 28 or 26 kDa as expressed selectively in HEK293 or NBL cells.

FIG. 45 shows that CA8 204^{C} inhibits forskolin induced phosphorylation of pITPR1.

### DETAILED DESCRIPTION

In various aspects, the disclosure relates to materials and methods, which provide safe and effective analgesia. This disclosure is the first to show that Carbonic anhydrase 10 (CA10 (human) and Car10 (rodent)) produces analgesia and prevents hyperalgesia, e.g., in chronic neuropathic and inflammatory pain models. In at least one aspect, the materials and methods relieve pain with minimal interference with motor and other sensory functions, thereby improving quality-of-life while minimizing the need for opioid use.

In one aspect, the disclosure provides a method of treating or preventing pain in a subject in need thereof. The method comprises administering to the subject an expression vector comprising a nucleic acid sequence encoding carbonic anhydrase 10 or carbonic anhydrase 11 such that the nucleic acid is expressed to produce carbonic anhydrase 10 or carbonic anhydrase 11 in the subject. In an alternative embodiment, the method comprises administering to the subject an expression vector comprising a nucleic acid sequence encoding a fragment of carbonic anhydrase 8 such that the nucleic acid is expressed to produce the fragment of carbonic anhydrase 8 in the subject. The nucleic acid sequence encoding fragment of carbonic anhydrase 8 comprises the first three exons of CA8, and optionally the nucleic acid sequence encoding the CA8 fragment is CA8-204 described herein. In various embodiments, the expression vector is a viral vector, such as an adeno-associated viral vector or a herpes simplex viral vector.

Aspects of the invention are described further below. The use of section headings are merely for the convenience of reading, and not intended to be limiting per se. The entire document is intended to be viewed as a unified disclosure, and it should be understood that all combinations of features described herein are contemplated.

### Carbonic Anhydrases

Carbonic anhydrase 10 (CA10) is a member of the carbonic anhydrase (CA) super gene family and one of three catalytically inactive CA isoforms. While CA10 retains a central carbonic anhydrase motif, it lacks the catalytic zinc coordinating residues critical for enzymatic activity. The functions of CA10 remain were previously unknown. Sequence comparison revealed 100% identity between humans (Homo sapiens), rat (Rattus norvegicus), and mouse (Mus musculus) CA10 proteins, and 90% identity at the amino acid level with zebra fish (Danio rerio). There are nine transcripts encoding human CA10, resulting in seven functional isoforms. Nucleic acid and amino acid sequences of human CA10 are set forth in Genbank Accession Nos. NM_020178 (SEQ ID NO: 3); NP_064563 (SEQ ID NO: 4); NM_001082534 (SEQ ID NO: 5); NP_001076003 (SEQ ID NO: 6); NM_001082533 (SEQ ID NO: 7) and NP_001076002 (SEQ ID NO: 8); the amino acid sequence of human CA10 is also provided in UniProtKB Q9NS85 (SEQ ID NO: 9).

In various aspects, the expression vector comprises a nucleic acid sequence encoding a peptide comprising at least 90% identity (e.g., at least 95% identity or 100% identity) to SEQ ID NO: 2. In various aspects, the expression vector comprises a nucleic acid sequence having at least 90% identity (e.g., at least 95% or 100% identity) to SEQ ID NO: 1. As used herein, “at least 90% identity” and similar terms encompass any integer from, e.g., 90% to 100%, such as 90%, 91%, 92%, 93%, 94%, 95%, 96%, 97%, 98% or 99% and the like. Also, the term “at least [percentage] identity” encompasses any percentage that is greater than or equal to the number of identical nucleotides or amino acids divided by the total number of nucleotides or amino acids ([at least percentage identity]≥[number of identical nucleotides or amino acids]/[total number of nucleotides or amino acids]). The calculation of percent identity of aligned amino acids (or nucleotides) of two or more sequences is well understood in the art and is determined conventionally using known computer programs. For example, alignment of two or more sequences to determine percent sequence identity is optionally performed using the algorithm described by Altschul et al. (Nucleic Acids Res., 25:3389-402 (1997)) as incorporated into BLAST (basic local alignment search tool) programs, available on the National Center for Biotechnology Information website. The gene product exhibits at least one carbonic anhydrase (CA10) activity, such as analgesia or antagonist of ITPR1-activation (pITPR1) and ITPR1-mediated intracellular calcium release.

It is surprising that CA10 and Car10 display the activities described herein (e.g., analgesia) despite the considerable sequence divergence between CA8 and CA10. The amino acid sequence of CA10 demonstrates an overall percent identity of only 25-57% to other CA isozymes, with the highest percent identity to a CA1l. Specifically, CA10 (NP_001076003.1) exhibits only about 33% identity with CA8 (NP_001308766.1) at the amino acid level. Indeed, CA10 most closely resembles CA11 compared CA8 or any other family member at the amino acid level, with CA10 demonstrating sequence identity of 58% with CA11 and 33% with CA8. CA10 lacks two out of three zinc-ligand binding histidine residues, suggesting a lack of CA enzymatic activity. CA11 also lacks zinc-ligand binding sites, suggesting a lack of enzymatic activity. Similar to CA8, CA10 was found to inhibit forskolin-induced phosphorylation of ITPR1 (pITPR1); and ATP-induced ITPR1 mediated intracellular calcium release in HEK293 and NBL cells. CA8 is thought to be an allosteric inhibitor of IP3 ligand binding and activation of ITPR1 leading to intracellular calcium release. Allosteric inhibition of ITPR1 activation was previously believed to depend on CA8 binding with ITPR1 (Hirota J, et al., Biochem J 372, 435-441). In distinct contrast to CA8, CA10 and Car10 do not bind ITPR1 in IP-westerns. Surprisingly, despite the lack of binding with ITPR1, CA10/Car10 and CA8-204 inhibit ITPR1 activation (pITPR1) and ATP induced intracellular calcium release, and produce profound analgesia in vivo.

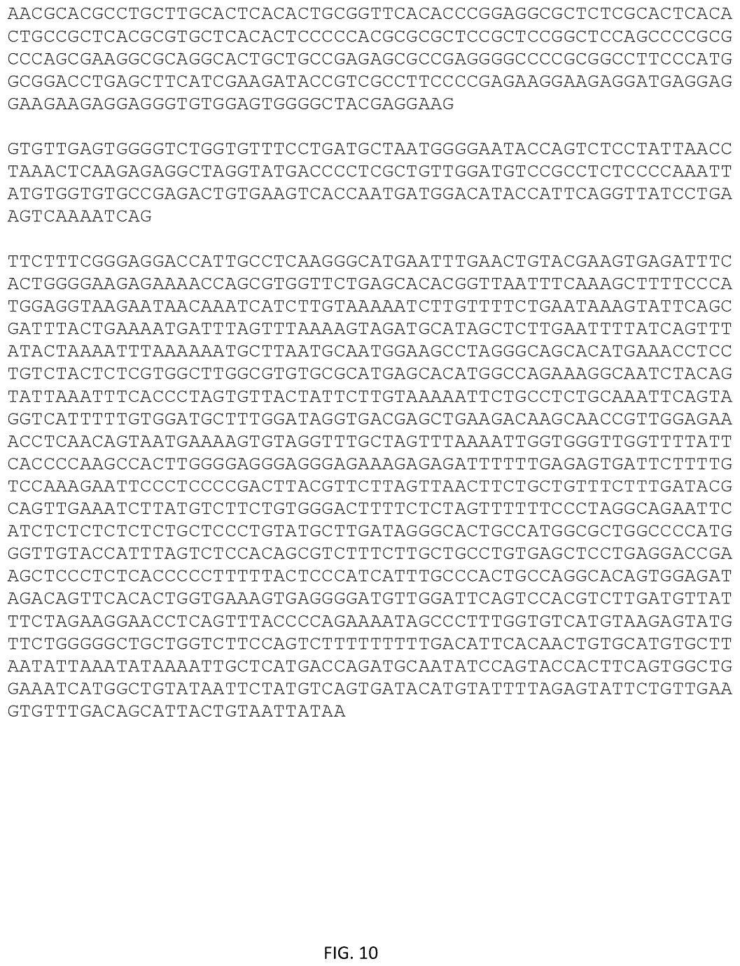

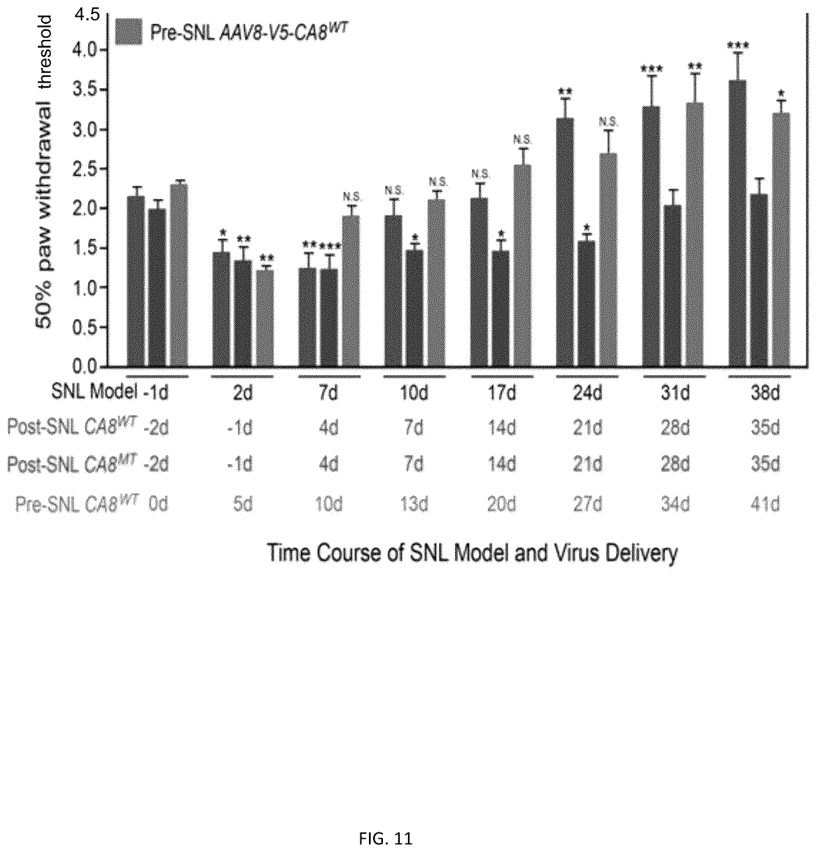

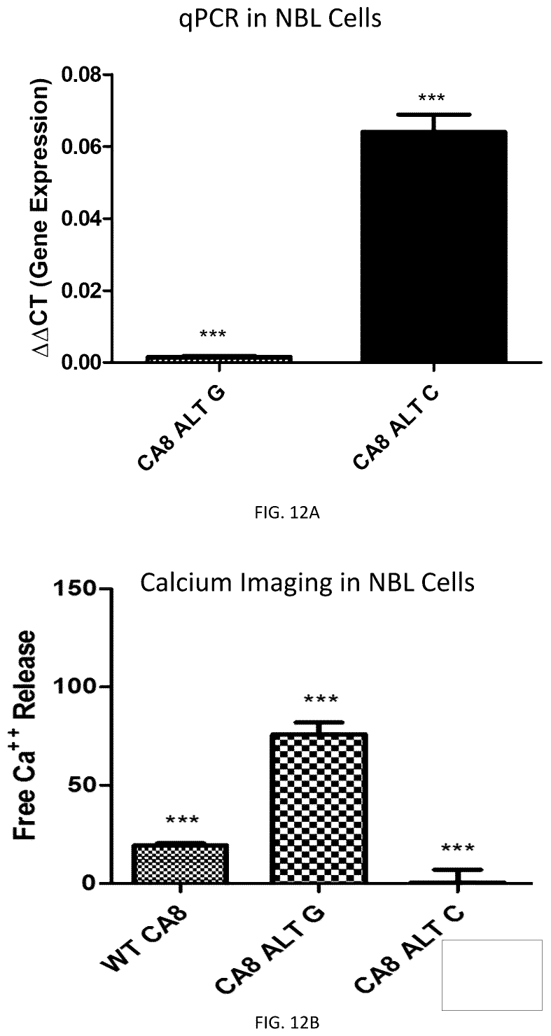

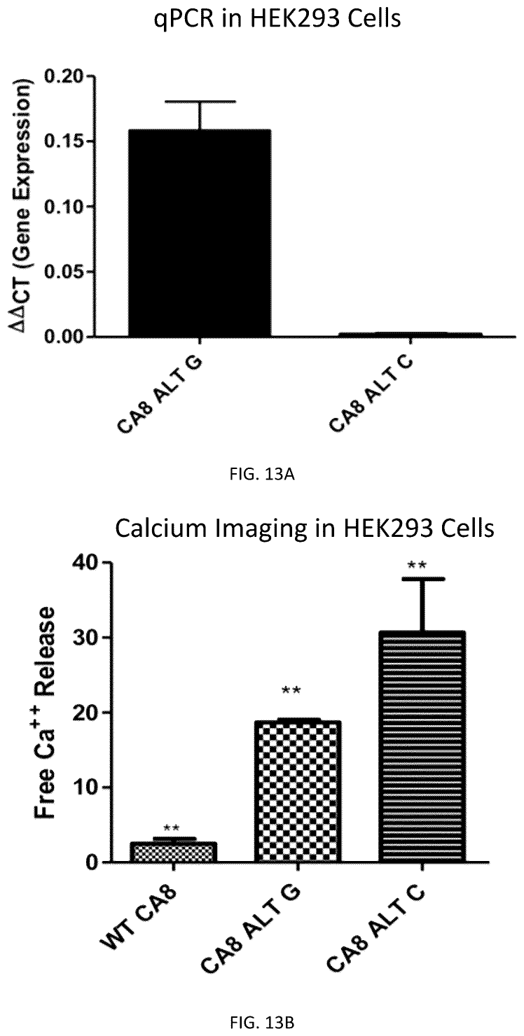

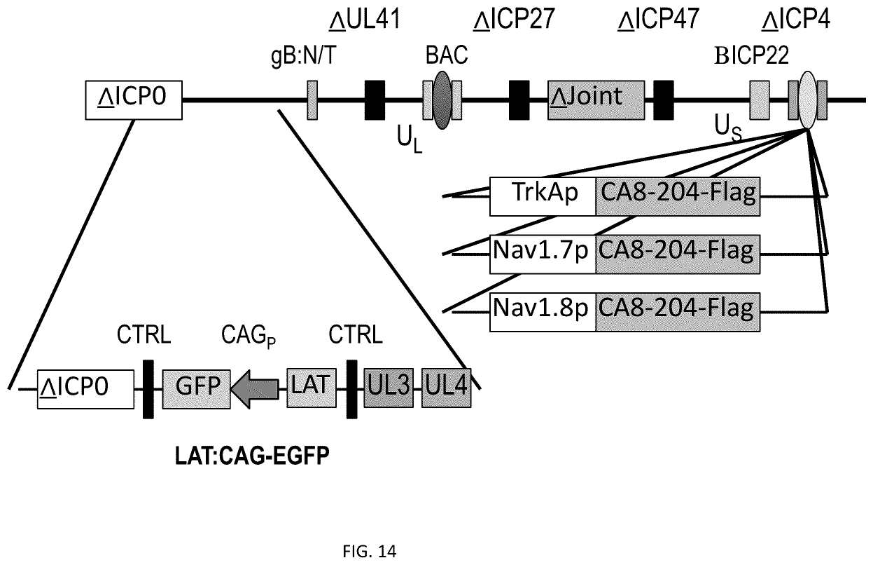

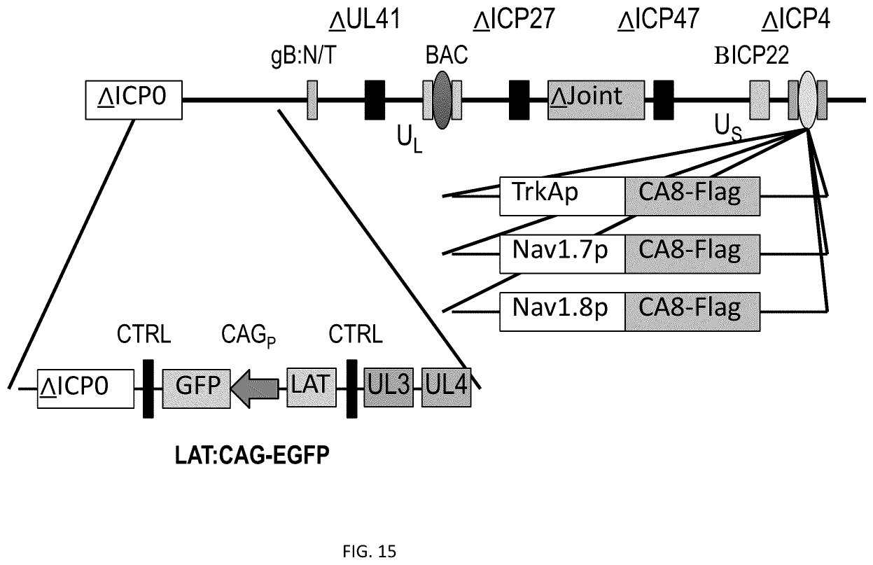

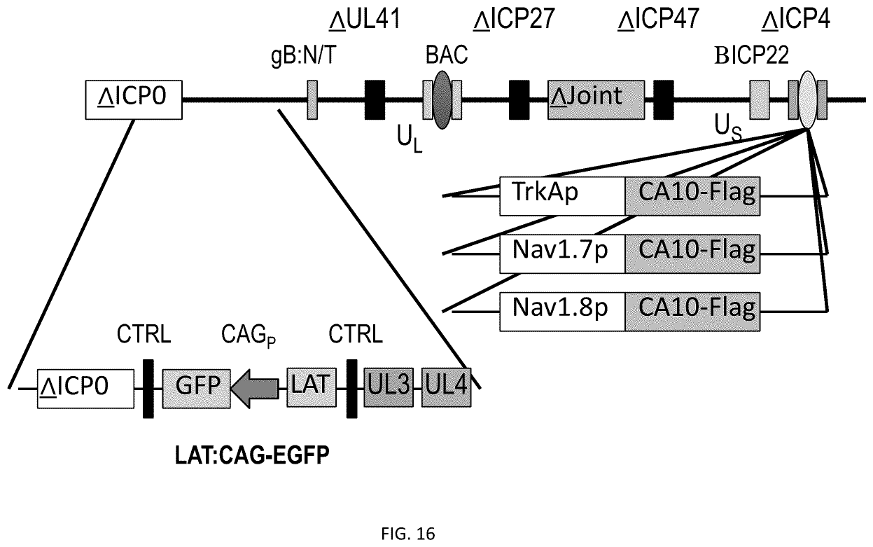

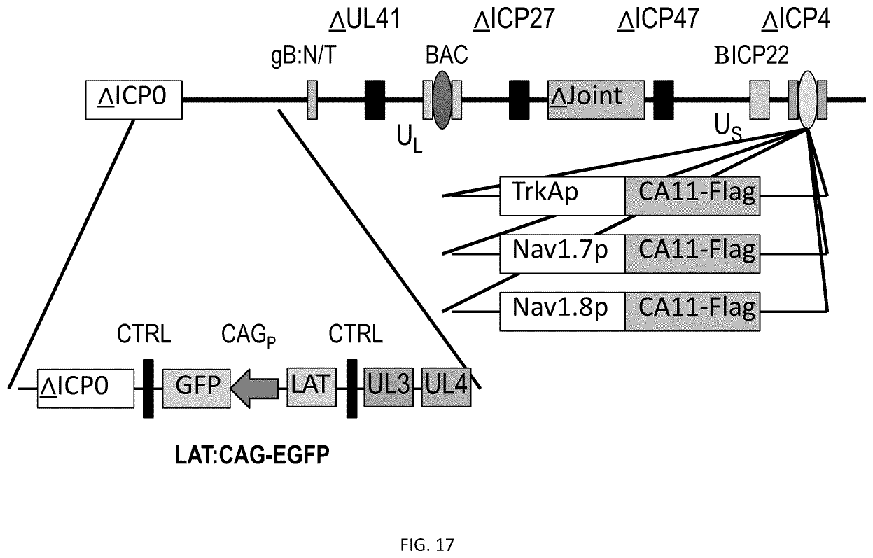

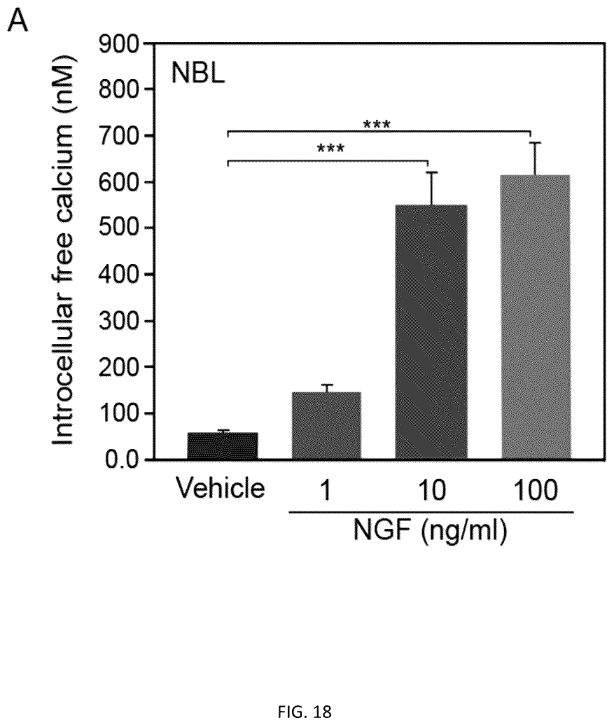

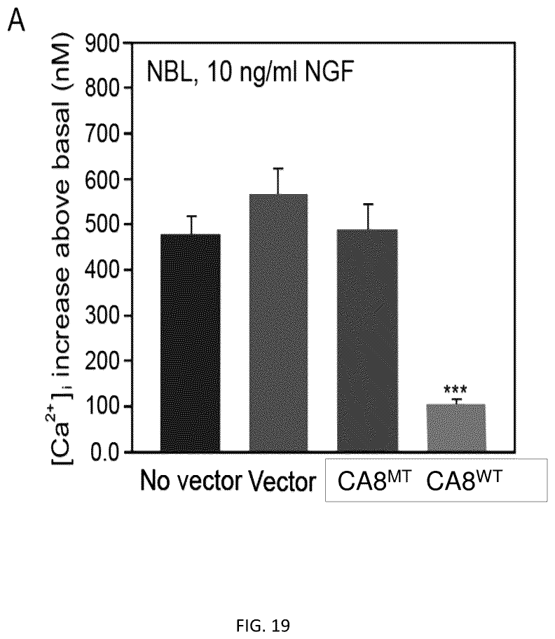

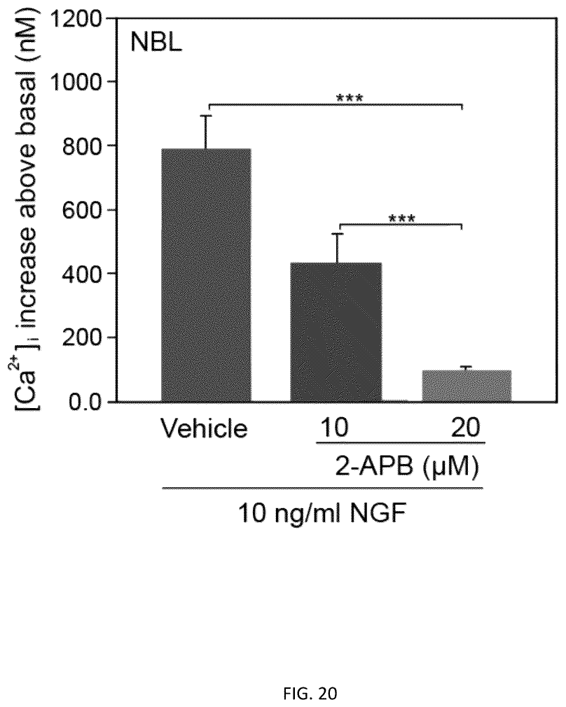

In various aspects, the expression vector comprises a nucleic acid sequence encoding a CA8 fragment. The nucleic acid sequence comprises (or consists of) the first three exons of the CA8 (or Car8) coding sequence. Optionally, the nucleic acid sequence encoding the CA8 fragment is CA8-204, the sequence of which is provided herein in FIG. 10. In some embodiments, the CA8 fragment is CA8-204C, the nucleic acid sequence of which is set forth in SEQ ID NO: 32 (the amino acid sequence is set forth in SEQ ID NO: 30). In some embodiments, the CA-fragment is CA8-204G, the nucleic acid sequence of which is set forth in SEQ ID NO: 33 (the amino acid sequence is set forth in SEQ ID NO: 31). In some embodiments, the CA-8 fragment is CA8-202 (SEQ ID NO: 34) or CA-203 (SEQ ID NO: 35). In various aspects, the expression vector comprises a nucleic acid sequence having at least 90% identity (e.g., at least 95% or 100% identity) to a CA8-204 nucleic acid sequence described herein. As used herein, “at least 90% identity” and similar terms encompass any integer from, e.g., 90% to 100%, such as 90%, 91%, 92%, 93%, 94%, 95%, 96%, 97%, 98% or 99% and the like. The nucleic acid sequence of a CA8 fragment comprising exons 1-3 is provided as SEQ ID NO: 1. The nucleic acid sequence of a CA8 fragment comprising exons 1-5 is provided as SEQ ID NO: 35. The nucleic acid sequence of a CA8 fragment comprising exons 1-8 is provided as SEQ ID NO: 34. The nucleotide and amino acid sequences of CA11 are provided as SEQ ID NOs: 10 and 11. The expression product of any of the sequences described herein exhibits at least one carbonic anhydrase activity, such as analgesia or antagonist of ITPR1-activation (pITPR1) and ITPR1-mediated intracellular calcium release.

Descriptions of materials and methods concerning CA8, CA10, and CA11 also apply to the mouse (version of the proteins, Car 8, Car10, and Car11, which are contemplated for use in various aspects of the disclosure.

### Expression Vector

A “vector” or “expression vector” is any type of genetic construct comprising a nucleic acid (DNA or RNA) for introduction into a host cell. In various embodiments, the expression vector is a viral vector, i.e., a virus particle comprising all or part of the viral genome, which can function as a nucleic acid delivery vehicle. Viral vectors comprising exogenous nucleic acid(s) encoding a gene product of interest also are referred to as recombinant viral vectors. As would be understood in the art, in some contexts, the term “viral vector” (and similar terms) may be used to refer to the vector genome in the absence of the viral capsid. Viral vectors for use in the context of the disclosure include, for example, retroviral vectors, herpes simplex virus (HSV)-based vectors, parvovirus-based vectors, e.g., adeno-associated virus (AAV)-based vectors, AAV-adenoviral chimeric vectors, and adenovirus-based vectors. Any of these viral vectors can be prepared using standard recombinant DNA techniques described in, e.g., Sambrook et al., Molecular Cloning, a Laboratory Manual, 2d edition, Cold Spring Harbor Press, Cold Spring Harbor, N.Y. (1989); Ausubel et al., Current Protocols in Molecular Biology, Greene Publishing Associates and John Wiley & Sons, New York, N. Y. (1994); Coen D. M, Molecular Genetics of Animal Viruses in Virology, 2^{nd} Edition, B. N. Fields (editor), Raven Press, N. Y. (1990) and the references cited therein. Additionally, viral vectors can be prepared with a large genomic coding sequence from humans and other host species, including an entire gene including 5′ and 3′ regulatory sequences with the application of homologous recombination-mediated cloning and manipulation of target genomic regions using Gateway cloning (Hartley et al., Genome Res 2000, 10:1788-1795) and/or “recombineering” systems (Copeland et al., Nat Rev Genet 2001, 2:769-779).

In various embodiments, a random or semirandom library is developed in which DNAs encoding precursors of carboxyl anhydrase peptides that differ are provided. Such a library may contain hundreds or more different sequences. In various aspects, thousands or more (at least 1000 or at least 10,000) different expression cassettes differing in the sequence of DNAs encoding the precursors of carboxyl anhydrase peptides constitute the library. Such a library can be constructed by first generating a population of random or semi-random oligonucleotides encoding precursors of peptides having one or more desired characteristics (e.g., precursors of carboxyl anhydrase peptides resembling CA8, CA10 or CA11). This population of oligonucleotides then can be cloned into the cassette backbone (i.e., in frame with the preproprotein signal sequence and optional biomarker).

An example of a method for constructing random or semi-random libraries employs the GATEWAY™ system (Invitrogen, Carlsbad, Calif.). In the GATEWAY™ system, ccdB is used as a negative selectable marker that, if present, kills the bacteria cell. ccdB is replaced by a random or semi-random sequence through site specific recombination carried out by a modified lambda integrase. Two bacterial strains are used in GATEWAY™ technology, ccdB sensitive and ccdB resistant. The ccdB containing plasmid is propagated in ccdB resistant bacteria and purified. This plasmid is then used for in vitro recombination. The recombination product is transformed into a ccdB sensitive bacteria selecting for plasmids that have had the ccdB gene replaced by the gene-of-interest during the in vitro recombination. By replacing ccdB, the background in cloning and library construction is dramatically reduced or eliminated allowing the shuttling of genes into and out or a variety of plasmids at will. As a starting point the base plasmids are grown in bacteria that are resistant to the toxic effects of ccdB of which there are a very limited number of genotypes available. To employ the GATEWAY™ technology in the context of this disclosure, using a large viral vector system, a bacterial strain amenable to transformation to large DNAs (such as BACs) desirably is modified to express a gene that confers insensitivity to ccdB. A preferred strain is derived from the DH10B bacterial strain used in BAC propagation and manipulation, which also has a mutation (fhuA::IS2) that increases their proclivity to transformation by very large DNAs.

In some embodiments, the viral vector is an HSV-based vector. HSV is an enveloped, icosahedral, double-stranded DNA virus that infects mammals, including humans. Wild-type HSV infects and replicates in both terminally differentiated non-dividing cells and dividing cells. An advantage of HSV vectors is the virus's ability to enter a latent stage resulting in long-term DNA expression. Additionally, HSV preferentially infects sensory nerves, often escapes immune surveillance, doesn't spread in the CNS/PNS, and exhibits superior retrograde transport (e.g., intradermal, intra-articular or peripheral nerve). Additionally, HSV allows for large genomic inserts using Gateway and/or recombincering techniques. The sequence of HSV is available at ncbi.nlm.nih.gov: 80/entrez/query.fcgi?cmd=Retrieve&db=nucleotide&list_uid-s=9629378&dopt=GenBank&term=hsv-1&qty=1.

Optionally, the HSV vector is replication-deficient, e.g., at least one HSV gene essential for replication or packaging is rendered non-functional (mutated or deleted). For instance, a replication-deficient HSV vector may lack one or more gene functions of the early regions, the immediate-early regions, or the late regions of the HSV genome. In various aspects, the HSV vector is “multiply-deficient,” meaning that more than one gene function essential for viral replication has been disrupted. For example, multiply-deficient vectors may lack gene functions from two or more of the early, immediate-early, and late regions of the HSV genome. The HSV vector optionally lacks a functional immediate early gene selected from the group consisting of ICP0, ICP4, ICP22, ICP27, ICP47, and any combination thereof, for example, lacks functional ICP0, ICP4, ICP22, ICP27, and ICP47 genes (optionally rendered non-functional by deletion). Non-essential genes also may be removed from a viral vector, such as an HSV vector, to accommodate large pieces of exogenous DNA. For example, an HSV vector can essentially lack the entire HSV genome. In this respect, the vector preferably comprises the viral inverted terminal repeats (ITRs) and/or the packaging signal, although these components are not required in all aspects of the disclosure. Optionally, one or more promoters or the viral ITRs and a packaging signal are left intact (i.e., an HSV amplicon).

The nucleotide encoding CA10 (or Car10) or CA1l (or Carl 1) optionally replaces native virus genetic sequences that have been removed (optionally to render the vector replication-deficient). The nucleotide encoding CA8 (or Car8) or CA8 fragment (or Car8 fragment) optionally replaces native virus genetic sequences that have been removed (e.g., to render the vector replication-deficient).

The HSV vector, when made replication deficient by the deletion of multiple genomic segments, optionally includes a spacer element to provide viral growth in a complementing cell line similar to that achieved by singly replication deficient HSV vectors. The spacer clement can contain any nucleic acid sequence or sequences that are of the desired length and encode the desired analgesic carbonic anhydrase molecule. The spacer element sequence can be coding or non-coding and native or non-native with respect to the HSV genome, but does not restore the replication essential function(s) to the deficient region. In addition, the inclusion of a spacer element in any or all of the deficient HSV regions will decrease the capacity of the HSV vector for large inserts.

In various embodiments, the HSV vectors are replication-defective HSV (rdHSV) vectors that are functionally deleted for all IE genes. An advantage to removing additional IE genes includes, but is not limited to, reducing toxicity in neurons and other cell types. The structure of a representative vector backbone comprises transgene cassettes inserted at, for example, two selected sites in the latency (LAT) locus that are protected against epigenetic silencing by resident insulator/chromatin boundary elements (CTRLs or CTCFs). These elements, along with the HSV LAP2 promoter, provide for long-term expression. The placement of an ectopic insulator adjacent to a transgene cassette inserted into the viral UL50-UL51 intergenic region also enhances prolonged transgene expression from this locus in primary human cells. As merely an example of a suitable vector system, HSV vector propagation reaching high titers has been demonstrated using a ICP4/ICP27/Cre-expressing U2OS cell line that eliminates the inhibitory BAC sequences present in vector constructs by Cre recombination.

It should be appreciated that the deletion of different regions of the HSV vector can alter the immune response of a host. In particular, the deletion of different regions can reduce the inflammatory response generated by the HSV vector. Furthermore, the HSV vector's protein coat can be modified so as to decrease the HSV vector's ability or inability to be recognized by a neutralizing antibody directed against the wild-type protein coat.

Base vectors, complementing cells, and engineering technology can generate safe vectors for long-term expression of CA10 (or Car10), CA11 (or Car11), CA8 (or Car8) and/or CA8 fragments (or Car8 fragments) for promoting analgesia from the following promoters within the Gateway transfer plasmid: sensory neuron specific Nav1.8 (e.g., sodium channel); neuron specific Nav1.7 (e.g., sodium channel); high affinity nerve growth factor receptor (TrkA); somatosensory-specific advillin CA8 driver; and the non-specific constitutive CMV promoter. Inducible promoter sequences, such as the tetracycline responsive promoter, also are appropriate in the context of the disclosure. Advantages of using HSV vectors to deliver human therapeutics include distinct tissue specificity, lack of immune response, and lack of latent reactivation, even in immunocompromised hosts.

In various aspects, the HSV vector comprises an HSV latency-associated transcript (LAT) insulator.

HSV-based vectors are further described in, for example, U.S. Pat. Nos. 5,837,532; 5,846,782; 5,849,572; and 5,804,413; as well as International Patent Publication Nos. WO 91/02788, WO 96/04394, WO 98/15637, and WO 99/06583, which are incorporated herein by reference in their entireties.

An example of an HSV vector for use in the context of the disclosure contains expanded ICP4, or ICP27 deletions, and preferably both. By “expanded” deletions is meant that the HSV vector contains no homologous sequences at either or both of these loci relative to the complementing cell line used for their production. Desirably, the virus has no remaining ICP4 or ICP27 (or both) coding or promoter sequences. Preferably, the deletion in ICP27 extends as well into the UL55 locus, and desirably both genes are deleted. Thus, a virus for use in the context of the disclosure contains extended deletions in ICP4, ICP27 and UL 55 such that there is no viral homology to these genes used in a complementing cell line. Desirably, the vector further does not include any homologous DNA sequences to that employed in the complementing cell line (e.g., even using different regulatory sequences and polyadenylation sequences).

It will be understood that vectors other than HSV-based vectors can be used in the context of the disclosure. For example, adenoviral, adeno-associated viral (AAV) and retroviral vectors are suitable for use in the methods and compositions of the disclosure. Construction of such vectors is known to those of ordinary skill in the art (see, e.g., U.S. Pat. Nos. 4,797,368, 5,691,176, 5,693,531, 5,880,102, 6,210,393, 6,268,213, 6,303,362, and 7,045,344). Non-viral methods can also be utilized for gene delivery and include, but are not limited to, gene-gun application of plasmids (e.g., non-viral expression vector encoding precursors of one or more carboxyl anhydrases described herein). Another non-viral method of gene delivery is electroporation. Alternative, implantable cell lines can be engineered to produce the desired peptide (or library).

In various aspects, the viral vector is an AAV vector. AAV is a DNA virus not known to cause human disease, making it a desirable gene therapy options. The AAV genome is comprised of two genes, rep and cap, flanked by inverted terminal repeats (ITRs), which contain recognition signals for DNA replication and viral packaging. AAV requires co-infection with a helper virus (i.e., an adenovirus or a herpes virus), or expression of helper genes, for efficient replication. AAV vectors used for administration of a therapeutic nucleic acid typically have a majority of the parental genome deleted, such that only the ITRs remain, although this is not required. Delivering the AAV rep protein enables integration of the AAV vector comprising AAV ITRs into a specific region of genome, if desired. Host cells comprising an integrated AAV genome show no change in cell growth or morphology. As such, prolonged expression of therapeutic factors from AAV vectors can be useful in treating persistent and chronic diseases. The AAV vector is optionally based on AAV type 1, AAV type 2, AAV type 3 (including types 3A and 3B), AAV type 4, AAV type 5, AAV type 6, AAV type 7, AAV type 8, AAV type 9, AAV type 10, or AAV type 11. The genomic sequences of AAV, as well as the sequences of the ITRs, Rep proteins, and capsid subunits are known in the art. Sec, e.g., International Patent Publications Nos. WO 00/28061, WO 99/61601, WO 98/11244; as well as U.S. Pat. No. 6,156,303, Srivistava et al. (1983) J Virol. 45:555; Chiorini et al (1998) J Virol. 71:6823; Xiao et al (1999) J Virol. 73:3994; Shade et al (1986) J Virol. 58:921; and Gao et al (2002) Proc. Nat. Acad. Sci. USA 99:11854.

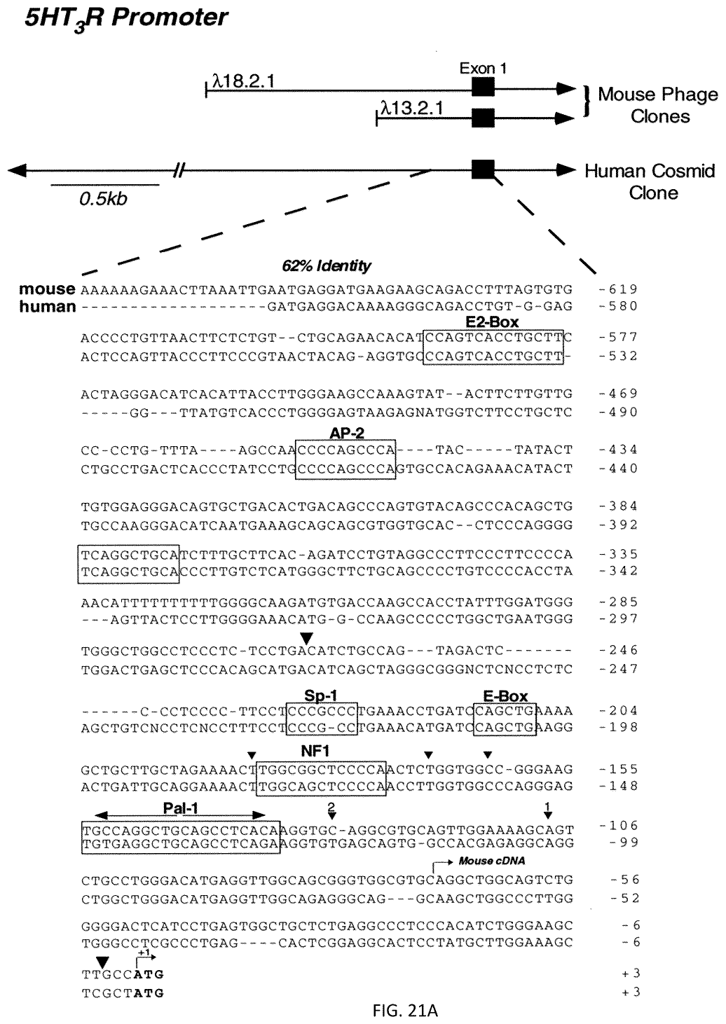

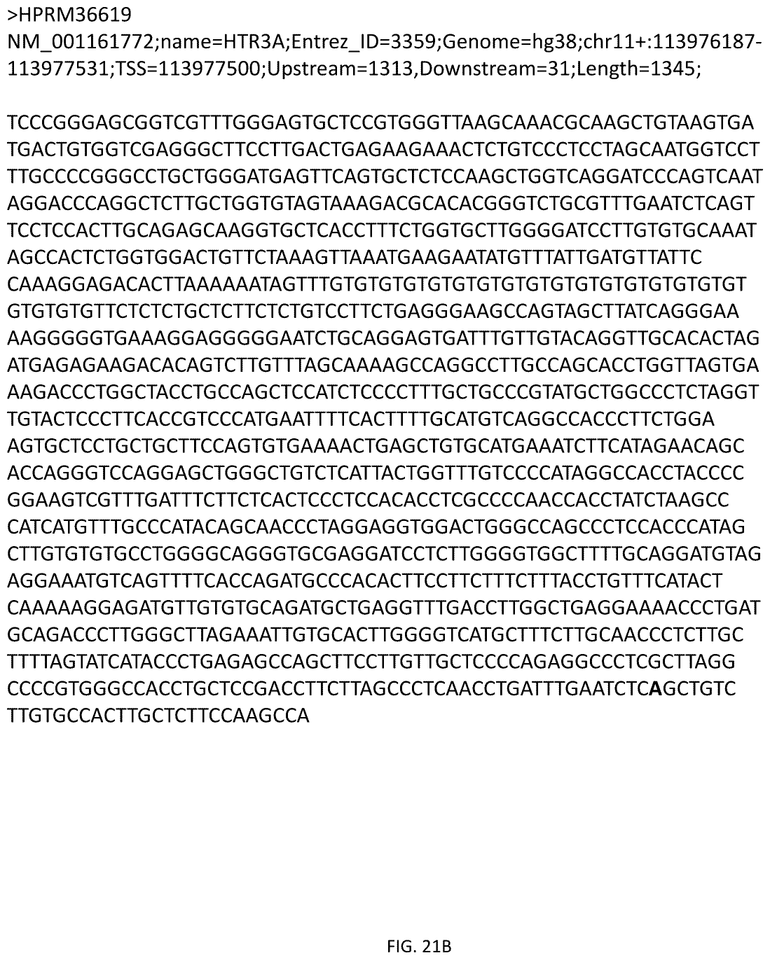

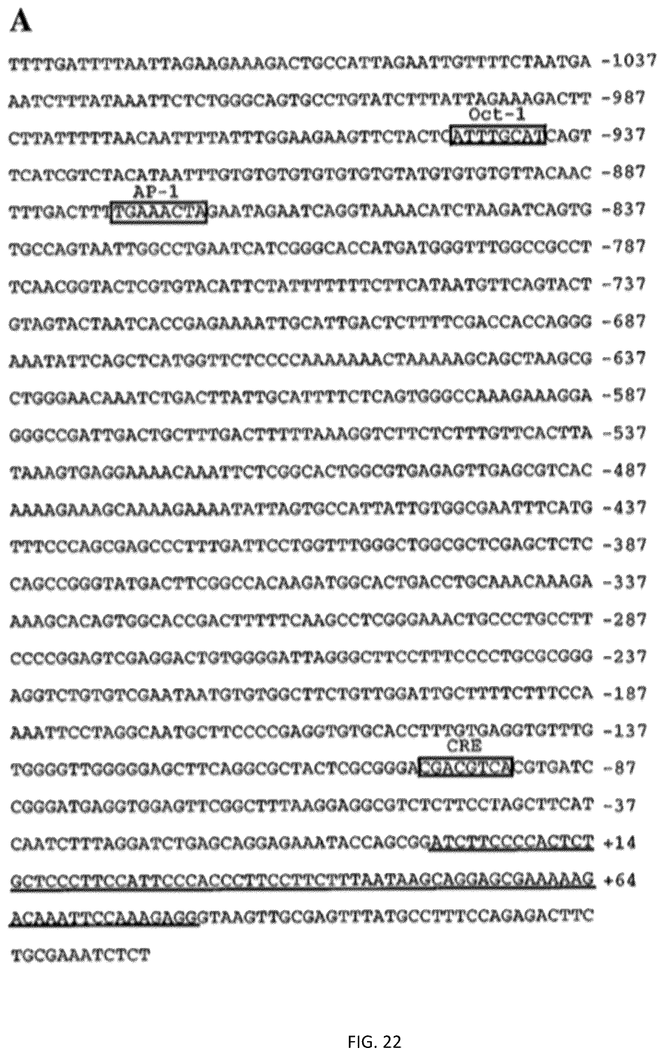

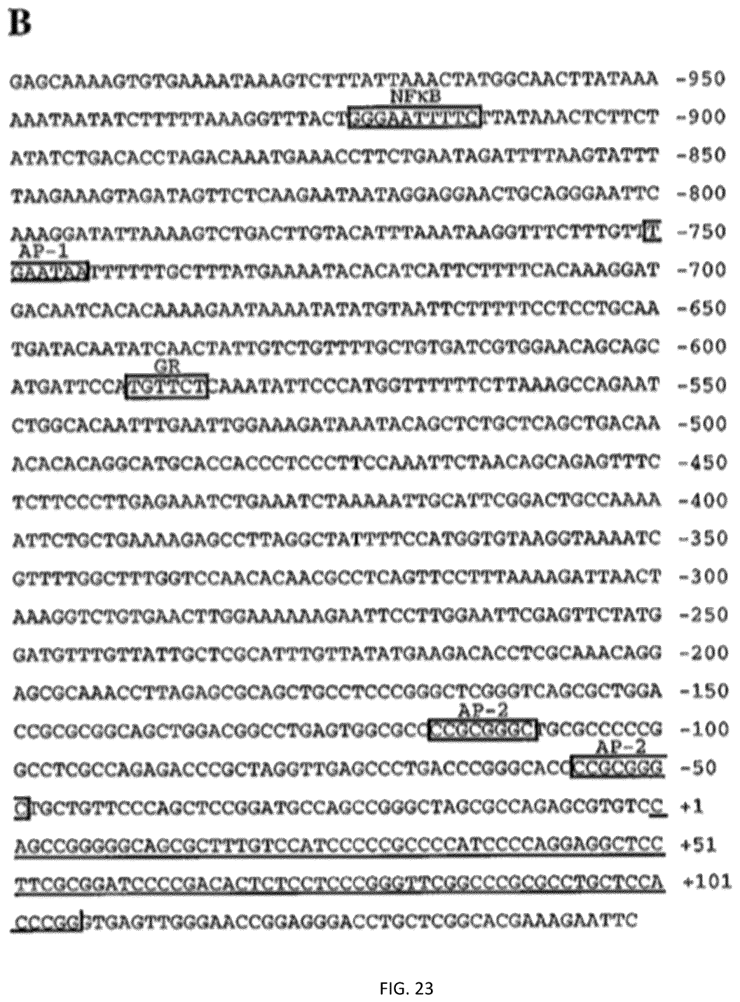

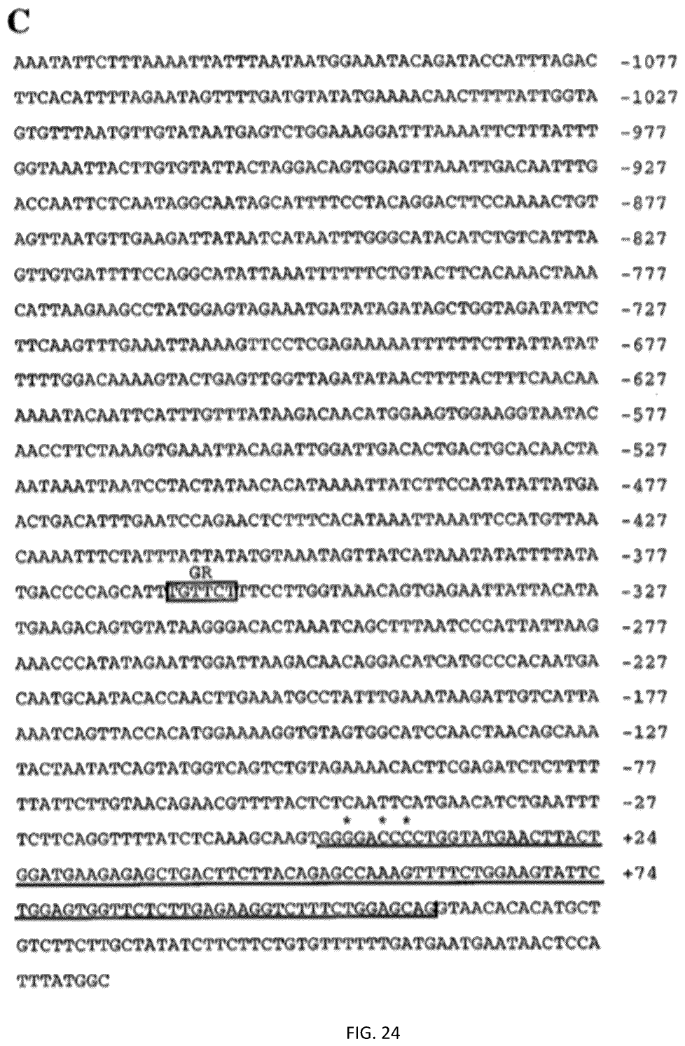

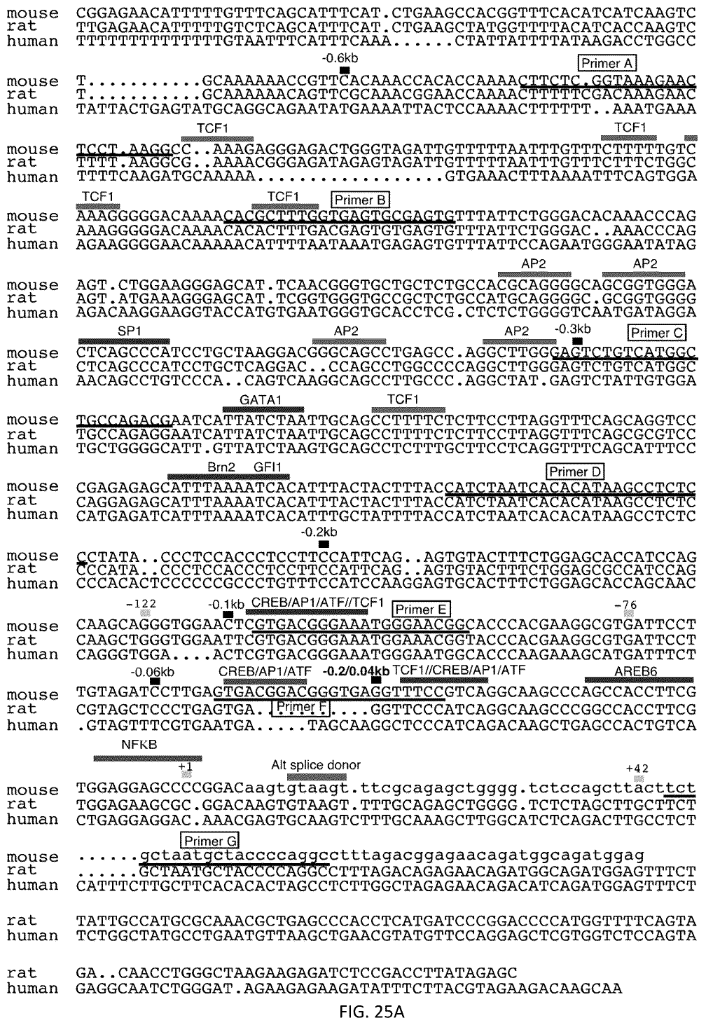

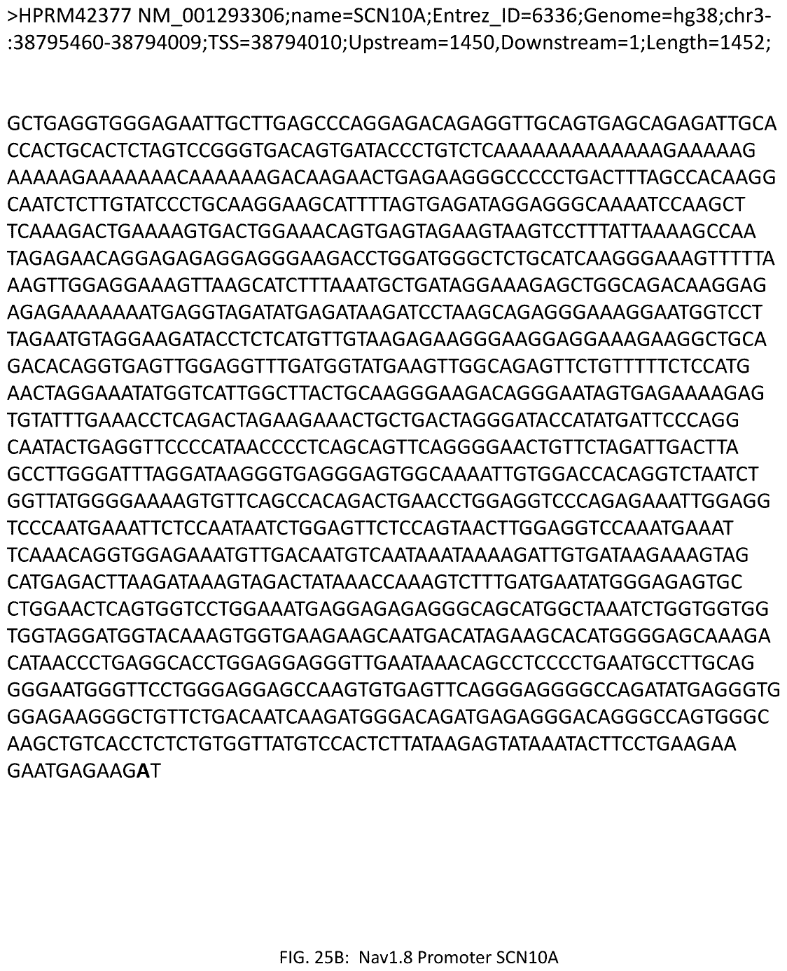

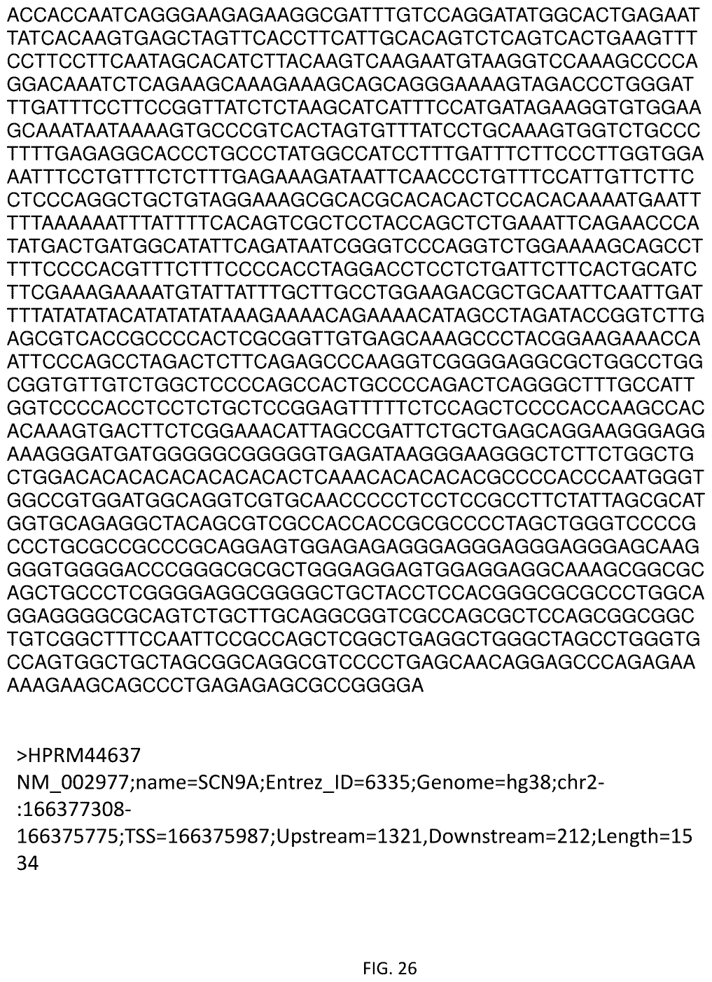

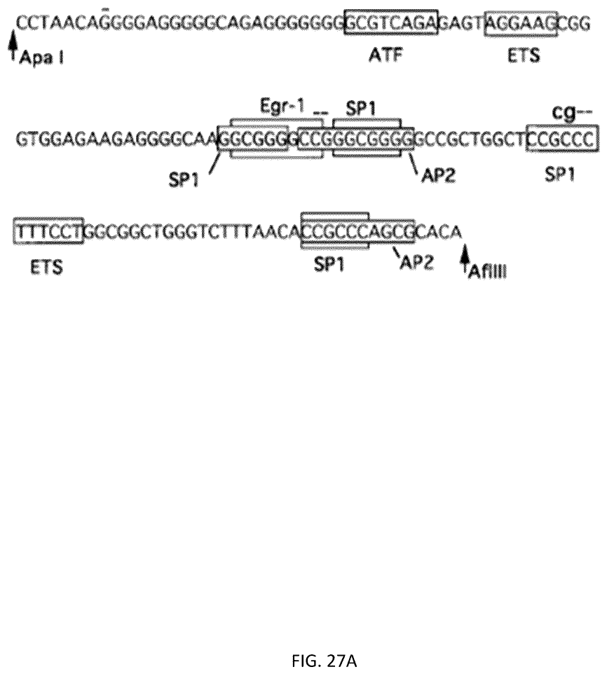

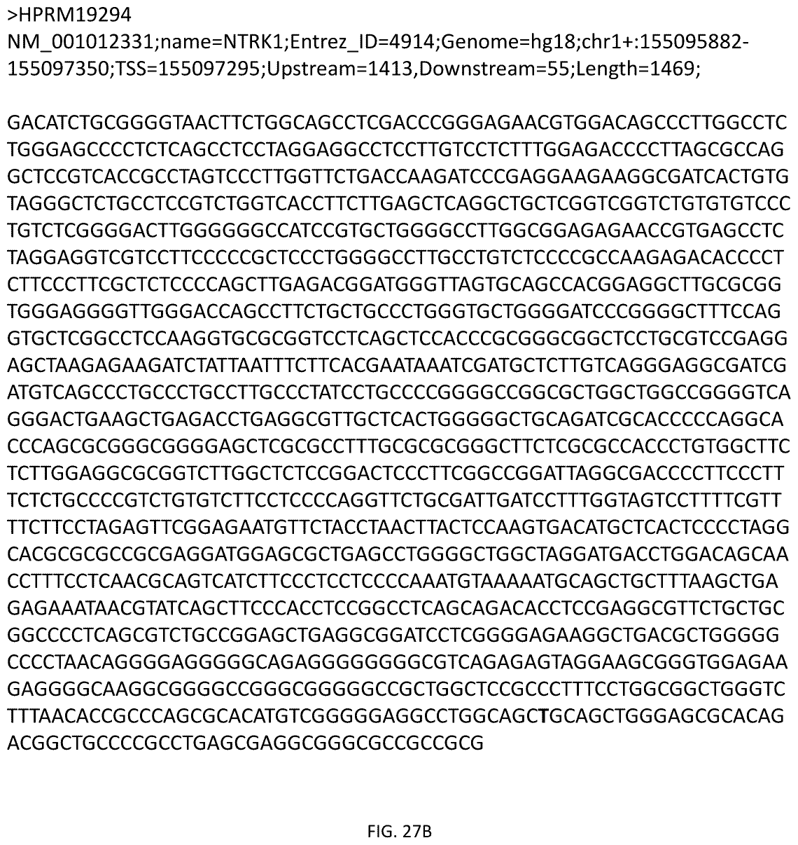

Expression vectors typically contain a variety of nucleic acid sequences necessary for the transcription and translation of an operably linked coding sequence. For example, expression vector can comprise origins of replication, polyadenylation signals, internal ribosome entry sites (IRES), promoters, enhancers, and the like. The vector of the disclosure preferably comprises a promoter operably linked to the CA10 (or Car10) or CA11 (or Carl1) coding sequence. In various aspects, the vector of the disclosure preferably comprises a promoter operably linked to the CA8 (or Car8) or CA8 fragment (or Car8 fragment) coding sequence. “Operably linked” means that a control sequence, such as a promoter, is in a correct location and orientation in relation to another nucleic acid sequence to exert its effect (e.g., initiation of transcription) on the nucleic acid sequence. A promoter can be native or non-native to the nucleic acid sequence to which it is operably linked and native or non-native to a particular target cell type, and the promoter may be, in various aspects, a constitutive promoter, a tissue-specific promoter, or an inducible promoter (e.g., a promoter system comprising a Tet on/off element, a RU486-inducible promoter, an ecdysone-inducible promoter, a rapamycin-inducible promoter, or a metallothionein promoter). For example, in various embodiments, an inducible promoter system is employed that allows the use of a small molecule to induce or stop production of analgesic peptide production. Examples of promoters include, but are not limited to, a sensory neuron specific promoter (such as the Nav 1.8 promoter), a somatosensory-specific promoter (such as the advillin promoter), the p175 promoter, or the TrkA (nerve growth factor receptor) promoter. Other promoters include, but are not limited to, TrkB, TrkC, Nav1.7, CGRP, ASIC3, NPY, NK1, 5HT, GRIN3A, or NF200 promoters. Non-limiting examples of sequences of various promoters are provided herein as FIGS. 21A-30.

Optionally, the virus coat or capsid (i.e., particle surface) is modified to adjust viral tropism. For example, the genome of one serotype of virus can be packaged into the capsid of a different serotype of virus to, e.g., evade the immune response. Alternatively (or in addition), components of the capsid can be modified to, e.g., expand the types of cells transduced by the resulting vector, avoid (in whole or in part) transduction of undesired cell types, or improve transduction efficiency of desired cell types. For example, transduction efficiency is generally determined by reference to a control (i.e., an unmodified, matched viral vector). Improvements in transduction efficiency can result in, e.g., at least about 25%, 50%, 60%, 70%, 80%, 85%, 90%, 95%, 100% improvement in transduction rate of a given cell type. If desired, the capsid can be modified such that it does not efficiently transduce non-target tissues, such as liver or germ cells (e.g., 50% or less, 30% or less, 20% or less, 10% or less, 5% or less of the level of transduction of desired target tissue(s)).

### Pain

“Pain” is generally described in terms of duration, cause, and/or afflicted region of the body. The invention includes treatment of any type of pain, including neuropathic pain (e.g., pain initiated or caused by a lesion or disease in the somatosensory nervous system), inflammatory pain (e.g., pain caused by activation and/or sensitization of the nociceptive pain pathway by inflammatory mediators), and/or nociceptive pain (e.g., pain caused by insult or injury of tissues). Many pain disorders or incidents are not easily classifiable, and may entail aspects or characteristics that overlap these general classes. Pain also is classified as to location in the body; somatic pain results from the activation of pain receptors in the body surface or musculoskeletal tissues, while visceral pain is felt in internal organs and is typically caused by the activation of pain receptors in the chest, pelvis, or abdomen.

Neuropathic pain occurs when there is actual nerve damage. Primary afferent somatosensory nerves represent the sensory nerves in the periphery and communicate with second order neurons in the spinal cord dorsal horn. Second order somatosensory nerves connect the spinal cord to the brain stem and third order neurons. Trauma (e.g., injury, surgery, toxic exposures, cancer, and/or metabolic or infectious diseases) can all damage the somatosensory pathway and cause spontaneous pain. Neuropathic pain can manifest as a burning, tingling, shooting, or stinging sensation, or be associated with more severe sensations including stabbing, piercing, cutting, or drilling. This type of pain typically occurs in waves of frequency and intensity, and is typically diffuse throughout portions or all of the body. Examples of neuropathic pain conditions include, but are not limited to, spinal cord injury-mediated pain, central pain syndromes (e.g., caused by a lesion within the nervous system), pain associated with peripheral nerve damage due to entrapment syndromes (e.g., carpel tunnel syndrome, cubital tunnel syndrome, or tarsal tunnel syndrome), multiple sclerosis, fibromyalgia, herpes zoster, virus-related neuropathies, painful traumatic mononeuropathy, polyneuropathy, diabetic neuropathy, post-surgical pain syndromes (e.g., post-mastectomy syndrome, post-thoracotomy syndrome, phantom pain), and complex regional pain syndrome (e.g., reflex sympathetic dystrophy and causalgia). The causes of neuropathic pain are numerous and include, e.g., chemical exposures (e.g., chemotherapy), trauma (e.g., amputation, disc herniation, or spinal cord injury), radiation exposure, metabolic disease, infection, and cancer.

Nociceptive pain is caused by damage to body tissues and is usually described as a sharp, aching, or throbbing pain. This type of pain can result from benign pathology, or by tumors or cancer cells that proliferate and crowd other body parts near the cancer site. Nociceptive pain may also be caused by cancer spreading to the bones, muscles, or joints, or blockage of an organ or blood vessels. Nociceptive pain may be associated with inflammation that includes, e.g., arthritic pain (such as rheumatoid arthritis or osteoarthritis) and inflammation-induced visceral pain (e.g., pain associated with inflammatory bowel disease, irritable bowel syndrome IBS, and the like). Examples of nociceptive pain include, e.g., pain from sprains, bone fractures, burns, bumps, bruises, inflammation (e.g., inflammation resulting from an infection, trauma, or arthritic disorder), or obstructions, as well as myofascial pain (which may indicate abnormal muscle or tendon stresses). Cancer pain can be nociceptive or neuropathic.

In various aspects, the method includes treatment of, e.g., long term, persistent pain, chronic pain, breakthrough pain, subacute pain, acute pain, and cancer pain. Acute pain is generally a limited physiological response to a discrete bodily insult (e.g., inflammation, surgery, bone fracture, headache, sprain, strains, burn, or chemical exposure). Acute pain generally lasts three to six months in duration. Chronic pain persists longer than would be expected for healing from a discrete bodily insult, e.g., more than three months. Chronic pain is associated with disorders such as back pain, trauma (e.g., surgery or wounds) causing nerve damage (including spinal cord injury), myofascial pain, arthritis, cancer-related pain, neuropathic pain, and fibromyalgia. In some embodiments, the pain involves acute-on-chronic pain, where acute pain flashes are superimposed on persistent, chronic pain.

Efficacy in treating (i.e., reducing, casing, suppressing, or alleviating) or preventing pain in a subject in need thereof is determined using any suitable method. In animal models, analgesic efficacy is measured, for example, using the tail withdrawal test, tail flick test, bee venom test, capsaicin test, or tail-clip test. Animal pain models are well characterized in the art and described in, e.g., Lariviere et al., Pain 97 (2002) 75-86. In humans, efficacy of (or need of) treatment is monitored or determined using, e.g., a pain score, time to re-medication, and quality of life measurements. Several tools are used in clinical settings to establish a numeric rating of pain intensity (see, e.g., McCaffery et al., (1989), Pain: Clinical manual for nursing practice, Mosby St. Louis, MO) or a verbal rating scale, which classifies pain as mild, moderate or severe. A reduced pain rating (intensity, frequency) by the subject, ability to resume activity, increased ability to sleep, and reduced need for pain medications are indicative of analgesia.

The disclosure provides a method of treating pain in a subject in need thereof. “Treating” pain does not require a 100% abolition of pain in the subject. Any decrease in pain sensation or symptoms constitutes a beneficial biological effect in a subject. In various aspects, the method reduces severity (which can include reducing need for and/or amount of (e.g., exposure to) other drugs and/or therapies generally used for these conditions), duration, and/or frequency of pain. “Preventing” pain does not require a complete preclusion of pain sensation; any dampening or delay of the onset of pain or associated symptoms is contemplated. In this regard, optionally, the expression vector is administered to the subject prophylactically, prior to onset of pain.

Various embodiments of the disclosure allow targeting of pain transmitting somatosensory nerves (e.g., Nav1.7; Nav1.8; Nav1.9; Trk-A; 5HT; and the like) to safely produce analgesia. The method, in some aspects, minimizes or avoids unwanted off-target effects, such as indiscriminant loss of desirable somatosensory and/or motor functions associated with other parenteral (anti-NGF) or ion channel inhibitors. Optionally, the expression vector is contained within a viral capsid (e.g., viral particle) and capable of long term expression of CA10 and CA11 (or CA8), minimizing the need for repeated, invasive interventions currently required for long-term management.

The disclosure further provides use of an expression vector comprising a nucleic acid sequence encoding carbonic anhydrase 10 or carbonic anhydrase 11 (or carbonic anhydrase 8) in the treatment or prevention of pain in a subject in need thereof; use of an expression vector comprising a nucleic acid sequence encoding carbonic anhydrase 10 or carbonic anhydrase 11 (or carbonic anhydrase 8) in the preparation of a medicament for treating or preventing pain in a subject in need thereof; and an expression vector comprising a nucleic acid sequence encoding carbonic anhydrase 10 or carbonic anhydrase 11 (or carbonic anhydrase 8) for use in the treatment or prevention of pain in a subject in need thereof.

### Analgesic Screening

The disclosure further provides a stock comprising a plurality of the gene transfer vectors encoding one or more candidate analgesic peptides. The stock can have any desired titer of vector, typically measured in plaque forming units (pfu) in the context of viral vectors. Typically the stock will have between about 10⁵ pfu/ml to about 10⁸ pfu/ml. In some embodiments, the stock is homogenous. In some embodiments, the DNA sequences encoding the candidate analgesic peptide(s) (or precursors thereof) differ between the vectors within the stock. In a various embodiments, respective DNA sequences encoding the candidate analgesic peptide(s) (or precursors thereof) among the vectors within the stock define a random or semi-random peptide library. Optionally, the DNA sequences encode precursors of carboxyl anhydrase peptides.

The disclosure provides a method for detecting a peptide having a desired analgesic property. A population of expression vectors described herein is introduced into a population of host cells (e.g., as NBL or HEK293 cells) under conditions suitable for expression of the encoded peptides. One or more host cells are then assayed for a desired effect representative of the desired analgesic property. If desired, the host cell(s) is assayed in comparison with a positive and/or negative control agent, such as those described herein for Car8/CA8. The control agent can be an agent known to precipitate the desired effect (positive control) or an agent known not to exhibit the desired effect (negative control). Optionally, the method further comprises deducing the DNA sequence encoding a peptide demonstrating the desired analgesic property.

The host cell(s) can be in vivo or in vitro. For in vitro applications, the assay is optionally conducted in multi-well plates (e.g., 96 well plates), which can facilitate high-throughput screening for desired pharmacodynamics and/or analgesic effect. For such applications, expression vectors from the library are optionally introduced into wells at a calculated titer of less than 1 vector per well (typically about 0.5 vectors per well) to minimize the statistical likelihood that more than one vector will transfect or infect the cells. In some embodiments, the expression vector is a viral vector, and in others, it is a plasmid or phage. Where a plasmid or phage (e.g., BAC) includes a viral genome, however, the cells within the wells will produce viral particles. Alternatively, a BAC system containing viral genomes (which comprise the respective DNA sequences and promoters) can be used to transform a larger number of cells, and viral particles rescued. The resultant viral particles then can be used in the assay. For example, if about 10,000 BACs containing HSV backbones that carry the random or semi-random library are introduced into host cells in a 6-well dish, after about 24 hours, about 100,000 viral particles typically can be harvested. These can be employed in the assay. Desirably, about 30,000 viral particles should be used (about three times the number of original vectors) to increase the likelihood that all members of the library are being assayed. The desired effect to be assayed can be any suitably measurable effect, such as apoptosis, antagonism of ITPR1 activation and calcium release or other aspects of this cell signalizing pathway, etc. Exemplary assays and methodologies are provided in the Example, which should not be construed to be limiting.

In some embodiments, the host cell(s) are in vivo (i.e., an animal model), which is particularly suitable when the desired effect to be assayed is behavioral in nature. For example, an analgesic effect can include a decrease in hyperalgesia or allodynia brought on by, for example, an external stimulus or a medical condition. In such embodiments, the library can be clonally expanded into a plurality of random stocks of vectors (each of which is substantially homologous), and the respective stocks introduced into an animal model of pain. The vector DNA from those stocks, which decrease the pain response in the animal, can then be sequenced to identify the encoded analgesic peptide.

### Formulations, Administration Regimens

In various aspects, the expression vector is provided in a composition (e.g., a pharmaceutical composition) comprising a physiologically-acceptable (i.e., pharmacologically-acceptable) carrier, buffer, excipient, or diluent. Any suitable physiologically-acceptable (e.g., pharmaceutically acceptable) carrier can be used within the context of the disclosure, and such carriers are well known in the art. The choice of carrier will be determined, in part, by the particular site to which the composition is to be administered and the particular method used to administer the composition. The composition also can comprise agents, which facilitate uptake of the expression vector into host cells. Suitable composition formulations include aqueous and non-aqueous solutions, isotonic sterile solutions, which can contain anti-oxidants, buffers, bacteriostats, and solutes that render the formulation isotonic with the blood, and aqueous and non-aqueous sterile suspensions that can include suspending agents, solubilizers, thickening agents, stabilizers, and preservatives. The composition can be presented in unit-dose or multi-dose sealed containers, such as ampules and vials, and can be stored in a freeze-dried (lyophilized) condition requiring only the addition of the sterile liquid carrier, for example, water, immediately prior to use. A composition comprising CA8-, CA8 fragment-, CA10- or, CA11-encoding expression vectors is, in one aspect, placed within containers, along with packaging material that provides instructions regarding the use of the composition. Generally, such instructions include a tangible expression describing the reagent concentration, as well as, in certain embodiments, relative amounts of excipient ingredients or diluents (e.g., water, saline or PBS) that may be necessary to reconstitute the composition.

In various embodiments, the expression vector is incorporated into lipid vesicles (which optionally enhances uptake) or provided in the form of a nanoparticle (e.g., by incorporation with a protein, lipid, carbohydrate, or combination thereof). Physical bombardment may be utilized to increase vector uptake by cells. Optionally, the expression vector is provided with chemical-based transduction enhancers. An example of a transduction enhancer includes lipoplex technology, wherein positively charged DNA is combined with anionic and neutral lipids to construct lipoplexes to enhance uptake. Polyplexes represent another form of chemical delivery complex for expressing units. In general, polyplexes consist of cationic polymers, and fabrication is based on ionic interactions and self-assembly. Another example, cationic liposomes, interact with cell membranes to enhance uptake through endocytosis. To improve transfection efficiency, electro-neutral lipids, such as DOPE are added to enhance release into the cytoplasm and escape lysosomal degradation. In another embodiment, polymersomes are used as an alternative to liposomes. Polymersomes are synthetic versions of liposomes (vesicles with a lipid bilayer) that tend to be more stable than liposomes, mechanically stronger, and have a longer storage self-life. Endosome-lytic agents include inactivated adenovirus that facilitate nanoparticle escape from the endocytic vesicle made during uptake.

Due to their low toxicity, greater carrying capacity, and case of fabrication, polycationic nanoparticles are an advantageous embodiment. Polyethyleneimine and chitosan are among the polymeric carriers suitable for expression vector delivery. Other polycationic carriers include poly (beta-amino esters) and polyphosphoramidate. Dendrimers are highly branched macromolecules with a spherical shape useful in aiding the cellular targeting of expressing units.

One embodiment includes the use of cationic dendrimers. These molecules naturally attract negatively charged genetic material such as DNA or RNA and this complex is taken into the target cells via endocytosis. Recently, dendrimers have been produced using kinetically driven chemistry that reduces cost and process time. “Priostar” dendrimers can carry a variety of expressing units including DNA or RNA and efficiently transfect target cells at a high efficiency with little or no toxicity.

Inorganic nanoparticles, such as gold, silica, iron oxide, and calcium phosphates represent another chemical means to deliver nucleic acid to target cells. Benefits of inorganic nanoparticles include stable prolonged storage, low cost manufacturing, minimal immunogenicity, and resistance to microbial attack. Nano-sized inorganic particles (e.g., less than 500 nm, preferably less than 250 nm, and most preferred less than 100 nm) represent another option for enhancing transduction, if desired. The nanoparticles can efficiently trap DNA or RNA and allow escape from the endosome without degradation.

Cell-penetrating peptides, also termed peptide transduction domains (PTDs), are short peptides (<40 amino acids) that efficiently pass through cell membranes while being covalently or non-covalently bound to various expressing units, facilitating their entry into cells. PTDs can be constructed to release exogenous nucleic acid to specific cell organelles by incorporating localization peptide sequences.

Some well-known physical methods of delivery of expressing units to target cells include the use of electroporation, sonoporation (ultrasonic frequencies to cavitate membranes making them more permeable to the expressing unit entry), magnetofection (expressing unit is complexed with magnetic particles enhancing the entry into target cells with a magnet), and hydrodynamic methods.

In various embodiments, the expression vector is incorporated into a viral capsid (viral particles) representing an infectious viral particle (including capsid, single or double stranded DNA, RNA, or other nucleic acid capable of coding for necessary peptide(s)), which can be advantageous to support latent infection and stable long-term analgesic peptide production. Peptide expression may be intracellular, and impact neuronal excitability and functioning in a way that produces analgesia or anti-hyperalgesia.

The expression vector (e.g., viral particle) is administered in an amount and at a location sufficient to provide some improvement or benefit to the subject, i.e., diminish or inhibit the sensation or perception of pain in the subject. Depending on the circumstances, a composition comprising the expression vector is applied or instilled into body cavities, applied directly to target tissue, and/or introduced into circulation. For example, in various circumstances, it will be desirable to deliver the composition comprising the expression vector by intravenous, intraperitoneal, intra-oral; intra-luminal (e.g., urinary bladder, gall bladder, bile ducts, pancreatic ducts, or sinus); intramuscular, intra-ocular, transcorneal, intraarterial, intraportal, intralesional, intradermal, intraarticular, intraneuronal, intraganglion, periganglion, intra-dermal, transdermal, subcutaneous, intraperitoneal, intranasal, inhalation (e.g., upper and/or lower airways), enteral, vaginal, or rectal means. In various aspects, the expression vector is administered directly to the pancreatic ducts, which is useful for, e.g., treating pain associated with pancreatic cancer or pancreatitis). In various aspects, the expression vector is administered to the trigeminal ganglia. If desired, the expression vector is administered regionally via intraarterial or intravenous administration feeding the region of interest. In various aspects, the expression vector described herein is administered directly or indirectly to peripheral somatosensory nerves. In one embodiment, the route of administration involves direct administration (e.g., injection or infusion) to dorsal root ganglion, other ganglia or somatosensory neurons, or the spinal cord. Optionally, the expression vector is administered via intra-articular injection or peripheral (e.g., sciatic) nerve injection. In various aspects, the expression vector is administered by intra-articular insertion to treat chronic nociceptive pain by, e.g., quieting the somatosensory nerves supplying an affected arthritic joint. In other embodiments, the expression vector is administered to various cavities, ducts, sinuses, or organs via a microcatheter or with direct visualization using an endoscope. Other embodiments include the use of needles to facilitate localization of expression vector to regions of pain. For example, the disclosure contemplates administration of the expression vector to sites (e.g., organ or other bodily site, such as joint) where pain arises using a catheter or needle. Still other embodiments include the use of imaging to guide the deposition of expression vector using for example, fluoroscopy, ultrasound, CT or MRI. A further embodiment includes the use of formulations that facilitate the delivery of expression vector via intradermal routes and to the gut by avoiding degradation in the stomach or upper gastrointestinal track.

In various aspects, enteric-coated encapsulation may be used to prevent degradation by gastric acid and inactivation of an expression vector. A formulation may include incorporation of a capsule composed of enteric-coated granules developed using Eudragit L30D-55 as a enteric polymer encasing expression vectors. Optimization of the capsule formulation may be achieved with an optimal protective coating with Eudragit L30D-55 demonstrating maximum viable vector count after two hours of incubation in acid medium and disintegration time of one hour in buffer pH 6.8. The amount of Eudragit L30D-55 in the capsules correlates with gastric juice resistance. Protective qualities against artificial gastric juice are observed when capsules were prepared from granules composed of vectors, corn starch, lactose monohydrate, polyvinylpyrrolidone and coated with 12.5% (m/V) of Eudragit L30D-55. Other coatings may be used to provide enteric-protective properties of a commercially available polymer EUDRAGIT®L100-55 on gelatin capsules and also on DRcaps®. Still other enteric coatings include, e.g., Vcaps® (Lonza) enteric coated capsules incorporating a polymer blend that enables effective delayed release, gastric protection, and protection of compounds with mild-to-moderate acid sensitivity; and enTRinsic Drug Delivery Technology incorporating capsule technologies described as a polymer blend that provides enteric protection to small and large molecules that are highly acid-sensitive.

Still other embodiments to provide gastric resistance to labile vectors can be also obtained by adding enteric polymeric systems to other dosage forms. Tablets, mini-tablets, pellets and granules (usually filled into capsule shells) are the most common enteric-coated dosage forms utilizing polymers noted elsewhere.

In various aspects, the expression vector is injected into a peripheral nerve (e.g., sciatic, femoral, infraorbital, trigeminal, facial, or suprascapular) or via intra-ganglion injection. In other embodiments, the expression unit maybe a naked single or double stranded DNA expression unit that is circular and resistant to nuclease destruction. In other embodiments, the vector may be incorporated into lipid vesicles for better absorption. Still other embodiments include single or double stranded DNA expressing units that are incorporated into a protein, lipid, carbohydrate molecules, or combinations of these as nanoparticles. Still other embodiments include physical methods of entry into target cells. An exemplary embodiment includes the use of chemical methods to enhance the uptake of expression vector entry into target cells. Other embodiments utilize a combination of physical, chemical, and biological methods for enhanced uptake of expression vectors into target cells.

Alternatively, the composition is administered locally via implantation of a membrane, sponge, or another appropriate material onto which the composition has been absorbed or encapsulated. Where an implantation device is used, the device is, in one aspect, implanted into a suitable tissue, and delivery of the expression vector is, for example, via diffusion, timed-release bolus, or continuous administration.

A particular administration regimen for a particular subject will depend, in part, upon the amount of therapeutic administered, the route of administration, and the cause and extent of any side effects. The amount administered to a subject (e.g., a mammal, such as a human) in accordance with the disclosure should be sufficient to affect the desired response over a reasonable time frame. In various embodiments of the method of treating or preventing pain in a subject, an expression vector encoding CA8 (or Car8), CA8 (or Car8) fragments (including CA8-204, CA8-204^{C}, CA8-204^{G}, CA8-202 and CA8-203), CA10 (or Car10) or CA11 (or Car11) is administered in an amount to induce analgesia. Put another way, the dose of composition administered is sufficient to reduce, case, suppress, or alleviate pain. Exemplary doses of viral particles in genomic equivalent titers of 10⁴-10¹⁵ transducing units (e.g., 10⁷-10¹² transducing units), or at least about 10⁵, 10⁶, 10⁷, 108, 10⁹, 10¹⁰, 10¹¹, 10¹², 10¹³, 10¹⁴, or 10¹⁵ transducing units or more (e.g., at least about 10⁷, 10⁸, 10⁹, 10¹⁰, 10¹¹, 10¹², 10¹³ or 10¹⁴ transducing units, such as about 10¹⁰ or 10¹² transducing units). Some conditions require prolonged treatment, which may or may not entail multiple administrations over time. Equivalent doses of vectors in genomic equivalents are 10⁴-10¹⁵, which can be quantified in vitro using quantitative PCR (qPCR) in term of expressing units (wherein an expressing unit is a discrete genetic unit capable of producing one peptide described herein). In various aspects, the dose comprises 10⁷-10¹² expressing units, or at least about 10⁴, 10⁵, 10⁶, 10⁷, 10⁸, 10⁹, 10¹⁰, 10¹¹, 10¹², 10¹³, 10¹⁴, or 10¹⁵ expressing units or more (e.g., at least about 10⁷, 10⁸, 10⁹, 10¹⁰, 10¹¹, 10¹², 10¹³, or 10¹⁴ expressing units, such as about 10¹⁰ or 10¹² expressing units).

When appropriate, the expression vector comprising a nucleic acid encoding CA8, CA8 fragment (including CA8-204, CA8-204^{C}, CA8-204^{G}, CA8-202 and CA8-203), CA10 or CA11 (or mouse versions thereof) is administered in combination with other substances (e.g., therapeutics) and/or other therapeutic modalities to achieve an additional (or augmented) biological effect. This aspect includes concurrent administration (i.e., substantially simultaneous administration) and non-concurrent administration (i.e., administration at different times, in any order, whether overlapping or not) of the expression vector and one or more additionally suitable agents(s). It will be appreciated that different components are, in certain aspects, administered in the same or in separate compositions, and by the same or different routes of administration.

According to a further aspect of the disclosure there is provided the use or method according to any other aspect of the invention wherein the CA8, CA8 fragment (including CA8-204, CA8-204^{C}, CA8-204^{G}, CA8-202 and CA8-203), CA10 or CA11 vector is administered separately, sequentially or simultaneously in combination with one or more agents useful for pain management. Examples of further agents include, but are not limited to, an opioid analgesic (e.g., morphine, hydromorphone, oxymorphone, fentanyl, codeine, dihydrocodeine, oxycodone, or hydrocodone); a nonsteroidal antiinflammatory drug (NSAID) (e.g., aspirin, diclofenac, ibuprofen, naproxen, oxaprozin, or cyclooxygenase-2 (COX-2) inhibitor); a sedative (e.g., a barbiturate sedative); a muscle relaxant; an antidepressant; an anticonvulsant (e.g., carbamazepine or valproate); an additional anesthetic; and a corticosteroid (e.g., dexamethasone).

The invention, thus generally described, will be understood more readily by reference to the following example, which is provided by way of illustration and is not intended to limit the invention.

### EXAMPLES

### Example 1

This example demonstrates that carbonic anhydrase-8 (CA8 human gene symbol, Car8 rodent ortholog); carbonic anhydyrase-10 (CA10 human gene symbol; Car10 rodent ortholog), and carbonic anhydrase-11 (CA11 human gene symbol; Car11 rodent ortholog), regulate the ITPR1-cytosolic free calcium-signaling pathway. ITPRs are believed to transduce signals arising from metabotropic receptor activation that generate inositol 1,4,5-trisphosphate (IP3) signaling molecules and intracellular calcium release from ITPRs, which play an important role in inflammatory pain behaviors. Zhuang et al., PLOS One, 2015. Data described herein for the first time show that CA8/Car8), CA8/Car8 fragments (including CA8-204); CA10/Car10, and CA11/Car11 function to inhibit IP3 activation of the ITPR1 calcium release channel, intracellular calcium release, and, surprisingly, inhibit analgesic responses to therapeutic analgesics (e.g., morphine and clonidine) by inhibiting intracellular calcium release, yet these CA peptides produce profound analgesia and treat or prevent chronic neuropathic pain in models. Also surprisingly, CA8/Car8 fragments (including CA8-204) and CA10/Car10 do not bind to ITPR1 (as demonstrated by co-immunoprecipitation); yet, in distinct contrast to CA8 (Car8) peptides, intracellular overexpression of CA8/Car8 fragments (including CA8-204) and CA10/Car10 peptides inhibit ITPR1 activation (phosphorylation) in response to forskolin and ATP-stimulated intracellular calcium release.

Additionally, data provided herein demonstrate for the first time that overexpression of CA8 selectively inhibits nerve growth factor (NGF) that signals nearly exclusively in NBL cells through ITPR1 as shown by near complete 2-APB. CA8 also inhibits NGF-induced ITPR1 activation (pITPR1), intracellular calcium release. Surprisingly, CA8 (Car8) DRG overexpression also is demonstrated herein to prevent and treat chronic neuropathic pain after spinal nerve (SNL) root injury. The following Examples establish that DRG CA8 (Car8) and CA10 (Car10) transduction and overexpression of the CA8 (Car8) and CA10 (Car10) protein down regulates ITPR1 activation (e.g., pITPR1 at Ser-1755) and inhibits intracellular calcium release. Surprisingly, despite the lack of amino acid homology between CA8 and CA10 (Car10) proteins, they also produce profound analgesia after sciatic nerve injections preventing mechanical allodynia and thermal hyperalgesia in chronic inflammatory and neuropathic pain models (spinal nerve ligation (SNL)).

Morphine produces analgesia by triggering the release of intracellular calcium. The data provided herein demonstrate that CA overexpression inhibits morphine induced ITPR1-mediated calcium release in NBL cells. Specifically, these data show that DRG Car levels are related to the half-maximal morphine and clonidine analgesic response. Higher Car expression is associated with higher morphine and clonidine half-maximal analgesic doses (Levitt et al., 2017). These data suggest for the first time that CAs may induce analgesic tolerance shifting the dose-response to clonidine and morphine to the right (e.g., higher doses of analgesic are required to produce the same amount of analgesic response), and these effects are related to inhibition of intracellular calcium release. This is unexpected, given that morphine requires intracellular calcium release to produce an analgesic response. Thus, it is surprising that CAs produce profound analgesia, instead of producing nociceptive pain by inhibiting the calcium regulatory pathway required for opioid and clonidine analgesic response.

### Materials and Methods

Animals: All experiments and procedures were performed according to the current guidelines for investigator of experimental pain in conscious animals, and were approved by the Animal Care and Use Committee of the University of Miami. Male adult C57BL/6 mice weighting 20-35 grams were obtained from Jackson Laboratories (Bar Harbor, ME) and were kept in a home cage environment with access to food and water ad libitum. Animals were housed in a 12-12h light-dark cycle in a virus/antigen-free facility with controlled humidity and temperature. Animals were allowed to acclimatize for 7 days before surgery and familiarize with the experimental equipment before testing.

Generation of viral constructs: As an example, a methods of generating adeno-associated vectors expressing mouse or human V5-Car10 or V5-CA10 proteins is described. Car10 (mouse) and CA10 (human) cDNA were purchased from ATCC. These gene products were amplified by Eppendorf Recycler gradient (Model 5331) and cloned between the BamHI and Xhol (NEB) restriction sites of the pcDNA3.1/V5-His A (Invitrogen™ Life Technologies, Carlsbad, CA) using the forward primer: TTTGGATCCGCCACCATGGCT-GACCTGAGCTTCATTG and the reverse primer: TTTCTCGAGCTGAAAGGCCGCTCGGA-TG. The V5-Car10 or V5-CA10 constructs were then amplified from pcDNA3.1/V5-His A and cloned between the BamHI and BglII restriction sites of the pAAV-MCS vector, one component of AAV Helper-Free System (Agilent Technologies, Santa Clara, CA) using the forward primer: CTCGGATCCGCCACCATGGC and the reverse primer: CTCGGATCCGCCA-CCATGGC.

Recombinant AAV8-V5-Car10 and AAV8-V5-CA10 viral particles were produced. Briefly, the vector plasmids, and the packaging plasmid AAV8 733 (5) and pHelper (Agilent Technologies, Santa Clara, CA) were co-transfected into HEK293 cells at 70% confluence using calcium phosphate precipitation method. The cells were incubated for 48 hours at 37° C. and 5% CO₂. After 48 hours, the cells were collected and freeze-thawed three times to release the AAV particles from the cells. After 30 min of Benzonase® Nuclease (Sigma) treatment, the crude lysate was clarified by low speed centrifugation. The supernatant was loaded on discontinuous iodixanol step gradients in OptiSeal™ tubes (Beckman Coulter) and centrifuged in a Type 70 Ti rotor (Beckman Coulter) at 69,000 rpm (350,000g) for 1 h at 18° C. The fraction containing AAV particles was collected and further purified using an AKTA FPLC system (GE Healthcare) by column chromatography on a 5 ml HiTrap column (GE Healthcare). About 25 mL was eluted from the column using elution buffer (20 mM Tris, 215 mM NaCl, pH 8.0), and the AAV particles were concentrated and buffer exchanged to 200 μl in HBSS (Invitrogen) using an Amicon Ultra-15 50K concentrator (Millipore). The purified AAV particles were then titrated for genome contents (expressing units) using qPCR methods. Titers in the range 1-3×10¹⁴ GC (Genome Copy) per mL were obtained.

Cell culture and transfections: Human neuronal-derived (e.g., NBL), human non-neuronal derived (e.g., HEK293) cells, or dispersed rodent primary DRG cells can be used. HEK293 cells (cat#CRL-1573 ATCC Manassas, VA) were cultured in Dulbecco's modified Eagle's medium (DMEM-Glutamax cat#0566; Invitrogen) supplemented with 10% fetal bovine serum, FBS (cat#16140 ThermoFisher scientific, Waltham, MA) and 1% penicillin/streptomycin (cat#15140 ThermoFisher scientific, Waltham, MA). Cells were seeded in six-well plates at density of 1.0×10⁵ cells per well. The following day, cells were transfected with plasmids via lipofectamine LTX reagent and plus (cat#15338 ThermoFisher scientific, Waltham, MA). For each transfection 2 μg of AAV2-ITR (control), AAV2-V5-Car10, or AAV2-V5-CA10 vectors was used. In various embodiments of the disclosure described herein, 2 μg of expression vectors (including positive and negative controls) in instances when AAV2 is not the viral vector employed.

Sciatic nerve injection of expression vectors (adeno-associated AAV8-V5-Car10 and AAV8-V5-CA10 vectors): Mice were anesthetized by intraperitoneal injection of Ketamine, xylazine and acepromazine cocktail (VEDCO, Saint Joseph, MO). Following sciatic nerve exposure, about 1.5 μl viral particles of AAV8-null (1.36E¹³ viral particles, SL100832 SignaGen Laboratories Rockville, MD), AAV8-V5-Car10 and AAV8-V5-CA10 (1.06E¹⁴ viral particles and 1.66E¹⁴ viral particles, respectively) were injected into the sciatic nerve using a 35-gauge Nanofil needle (World Precision Instruments, Sarasota, FL). The sciatic injection site was approximately 45 mm from the tip of the third toc.

Inflammatory hyperalgesia models: Mice received a 30 μl intradermal injection of 1% carrageenan (cat#22049 Sigma, St Louis, MO) 2.5 mg/ml in sterile 0.9% saline or complete Freund's adjuvant, CFA (cat#F5881 Sigma, St Louis, MO) 0.5 mg/ml in sterile 0.9% saline into the left hind paw. Fehrenbacher et al., Curr Protoc Pharmacol. 2012; Chapter 5: Unit 54.

Neuropathic pain model: For the induction of peripheral neuropathy, mice were first anesthetized by an intraperitoneal injection of ketamine, xylazine hydrochloride and acepromazine (VEDCO, Saint Joseph, MO). Then, a tight ligation of the spinal nerve (left L5 in mouse model) was performed using a previous described procedure. Kim et al., Pain. 1992;50 (3): 355-363.

Behavioral tests: Thermal and mechanical sensitivity was measured by Hargreaves test and von Frey filament threshold calculations respectively. Sec, e.g., Boyce-Rustay et al. Methods Mol Biol. 2010;617:41-55; Hargreaves et al., Pain. 1988;32 (1): 77-88; and Chaplan et al., J Neurosci Methods. 1994;53 (1): 55-63. Tests were performed in a quiet room with daylight-like illumination. Animals were habituated to the behavioral room and apparatus for at least 60 minutes for 1 week before a blinded investigator collected data. The thermal sensitivity test was performed using an IITC Plantar Analgesia Meter apparatus (IITC Life sciences, Woodland Hills, CA) with a plastic box placed on a glass plate of constant temperature (30° C.). The mouse plantar surface was exposed to a beam of radiant heat to induced paw withdrawal. Baseline latencies were adjusted to 5-9 sec with a maximum of 20 sec as cutoff to prevent potential injury. The latency time in seconds from the onset of the intense light beam to paw withdrawal was defined as the withdrawal latency of the paw. Two consecutive tests were averaged to establish the paw withdrawal latency. The mechanical sensitivity test was performed in an inverted plastic box placed on an elevated mesh floor. The mouse hind paw was pressed with one of a series of von Frey filaments with logarithmically incrementing stiffness (Stoelting Co, Wood Dale, IL) presented perpendicular to the plantar surface of each hind paw for 1-2 seconds, the 50% threshold was determined using the Up-and Down method.

Pharmacodynamics—Bioassay of Calcium Release: Fifteen mm glass coverslips (cat#72228 Electron Microscopy Sciences, Hatfield, PA) were coated with poly-D-Lysinc (Sigma) followed by Laminin and 1×105 cells were seeded on each coverslip. Twenty-four hrs later, cells were transfected with AAV2-ITR, AAV2-V5-Car10, or AAV2-V5-CA10 vectors as previously described. In embodiments wherein AAV2 is not employed, other expression vectors (including viral vectors) are used. Fura-2AM (cat#F1221 ThermoFisher scientific, Waltham, MA) was dissolved in DMSO (50 μg in 50 μl) and 1% pluronic acid-127 (cat#P2443 Sigma, St Louis, MO) as the stock solution. Forty eight hrs after seeding, cells were loaded with 2 μM Fura-2AM dye for 45 min at room temperature in the dark in a standard Ca+2 buffer solution containing: 125 mM NaCl, 2 mM MgCl, 4.5 mM KCl, 10 glucose, 20 mM HEPES, 2 mM CaC12, pH 7.4.25. Following dye loading, coverslips were washed with Ca⁺² buffer solution. For imaging and ATP stimulation experiments, coverslips were placed in a recording chamber (cat#QR-42LP Warner instruments Hamden, CT) on a Leica DMI6000B microscope, and perfused at room temperature (˜22° C.) with Ca⁺² free buffer solution containing: 125 mM NaCl, 4 mM MgCl, 4.5 mM KCl, 10 mM glucose, 20 mM HEPES, pH 7.4. For the ratiometric imaging of Fura-2AM, the excitation light was filtered through an ultra high-speed wavelength switcher to provide wavelengths of 340 and 384 nm and capture by a high-speed digital camera (Leica DFC365FX). Activation of ITPR1 channels was achieved via application of 1 μM ATP (Sigma, St Louis, MO). Data acquisition and processing was made using the LAXS software. Regions of interest over the field of view were selected and the mean pixels intensity at each frame was measured. Data was plotted as ratio fluorescence intensity versus time and subsequently converted to a relative scale.

Immunoprecipitation: Fifty ul magnetic beads (Invitrogen) were incubated with lug pITPR1 antibody for 45 minutes at 4° C. The supernatant was discarded and the beads were washed with binding buffer. Sample proteins of 200-400 μg were added and incubated with the beads for 4 hrs at 4° C. Then the protein complex was washed 3 times with washing buffer, and then eluted with SDS sample buffer. Samples were then heated at 70° C. for 10 min and then subjected to western blot analysis for Car10. The method described herein also is suitable for use in connection with Car8, CA8, CA8 fragment (CA8-204), CA10, Car11 and CA11.

Immunohistochemistry (IHC): HEK293 cells were seeded in poly-L-Lysine/laminin coated glass coverslips at a density of per coverslip, 24 hrs later cells were transfected with Car10, CA10 or empty vector respectively. Cells were cultured at 37° C. for an additional 96 hrs, then treated with forskolin 1 μM (F6886 Sigma, St. Louis, MO) for 5 min, then fixed with 4% paraformaldehyde for 15 min, permeabilized with 0.1% Triton X-100 for 10 min, and blocked with 1% bovine serum albumin (BSA, sigma) for 1 h. Cells were then incubated with anti-pITPR1 (cat#8548s cell signaling technology), anti-Car10(cat#SAB1102286 Sigma, St Louis, MO), anti-V5 (cat#R960 Invitrogen), anti-ITPR1 (cat# 8568 Cell Signaling technology) antibody overnight at 4° C. The next day, cells were incubated with the corresponding second antibody (1:200) for 1 h at room temperature in the dark. Coverslips were dried and affixed to slides using a fluorescent mounting medium containing Dapi (cat#P36931 Life Technologies). It will be understood that IHC methods also are contemplated using anti-Car8, anti-CA8, anti-CA8 fragment (CA8-204), anti-CA10, anti-Car11 and anti-CA11 antibodies.

Statistical analysis: Data was expressed as means ±standard error of the mean (SEM) and analyzed for statistical significant by Student's t test for two-group comparison, one-way ANOVA with Bonferroni's post hoc test for multiple comparison (three or more groups) with one variance, and two-way ANOVA with Bonferroni' s post hoc test for multiple comparisons (three or more groups) with two variances. All data analysis and graphics were performed using the GraphPadPrism 5.0 software (GraphPad Inc, San Diego, CA).

Pharmacodynamics: Bioassays of Electrophysiology Impact of Viral Constructs—validation of reduced neuronal excitability in vitro: Neurons and other transfected cells are studied in current clamp, perforated whole-cell configuration of the patch-clamp technique, at room temperature (20-25° C.). Perforation is obtained by amphotericin B to ensure satisfactory current clamp recordings, while maintaining intact cytosolic calcium concentration and pertinent cytosolic signaling apparatus in each cell population to be studied. Patch micropipettes (resistances 3-6 M) are pulled and polished, as described previously. Sarantopoulos C, et al., J Neurosci Methods. 2004;139 (1): 61-8; Sarantopoulos C, et al., Reg Anesth Pain Med. 2002;27 (1): 47-57; Kawano T, et al., Mol Pain. 2009;5:12; Sarantopoulos CD, et al., Brain Res. 2007; 1132 (1): 84-99; Hogan QH, et al., Pain. 2000;86 (1-2): 43-53. For recordings, a Multiclamp 700 B amplifier is used (Axon Instruments, Foster, CA, USA), and signals are digitized using a converter (DigiData 1440 A; Axon Instruments). The pCLAMP software (Axon Instruments) is used for analysis. Whole cell current clamp recordings are conducted using extracellular Tyrode's solution, and internal pipette solution, as described previously. Excitability parameters are compared between groups of cells classified by expression parameters. Recordings from cell populations differing by expression patterns (e.g., TrkA or Nav1.8 positive and negative), size (large vs. small DRG somatosensory neurons), and excitability differ depending on the viral vector are used. The following parameters are compared between groups: (1) Resting membrane potential (RMP) recorded at baseline for at least 3 min and spontaneous electrical activity (number of spontaneous action potential (AP) spikes/min). RMP for further comparison are determined after stable recording is established for at least 1 min. Neurons with a resting potential more depolarized than −45 mV, indicating large leak current, are rejected. (2) AP is evoked in response to supra-threshold stimulation by current injection. The current threshold is determined by sequential 25 pA step increments until a monomorphic AP is triggered, and cach threshold will be recorded. Then, an AP is elicited by a single, 2 ms supra-threshold current pulse, and captured in subsequent recordings lasting 500 ms (to measure both AP and after-hyperpolarization (AHP) parameters). (3) Characteristics of AP are measured and compared between groups including RMP, peak AP amplitude, AP threshold and AP duration at threshold, as well as at 5% and 50% amplitude. AP threshold is measured at the beginning of the sharp upward rise of the depolarizing phase. AP is also measured from the point where a horizontal diachronic line is drawn from this AP threshold to the point where the descending, repolarizing phase crosses this line. AP amplitude is measured from RMP to the AP peak. AP duration is determined at a voltage 5% from RMP to the AP peak, as well as at the midpoint of 50% voltage from RMP to peak. AP magnitude is also expressed as arca under the curve. AHP amplitude is measured from the RMP to the most hyperpolarized level of the AHP phase. AHP duration is measured at points representing 50% and 90% recovery back to RMP. AHP magnitude is also expressed as area under the curve. Two other measures of cell excitability are used including: rheobase (which is determined as the minimum current amplitude in a gradually stepwise increasing series of depolarizing 200-ms pulses that elicits an AP) and the pattern of AP spike generation during current injection steps of at least twice that of rheobase, at which cells either produce single APs or fire repetitively.

HSV TrkA and Nav1.8 Promoters Drive Robust Long Term Reporter Expression: Virus only reaches DRG cell bodies via retrograde transport in vivo. Since these are rdHSV, they cannot infect nearby glia or other DRG cells that don't project to the local injection site. After 30+ days, strong expression is observed from both the LAT and ICP4 loci in rodents. The ICP4 locus increases with time, which is why it was chosen as the site for the expression cassettes (TrkAp-CA, Nav1.8p-CA, Advillinp-CA, etc.). If, for example, sufficient expression in neurons is not demonstrated, or if expression decreases with time, expression cassettes can be relocated in the LAT locus.

Pharmacokinetics: Dose Response and Tissue Specificity—Structural and functional validation in vivo: Analgesia and motor function testing include measures of mechanical pain (von Frey), thermal pain (Hargreaves), and non-reflexive sensory and motor functions (voluntary wheel running using automated measures: wheel distance/time, wheel time; and stride). Eighteen naïve male C57BL/6J mice per assay condition is used. The initial time course of analgesic response is monitored every other day until D14 and then weekly until D28 (end-of-life point). Clinical safety assessments are made at baseline and weekly in each mouse (e.g., body wt., general appearance, food consumption, blood pressure, body temperature). Restricted neuronal expression is assessed as in these preliminary studies in skin, peripheral nerves, DRG and dorsal horn (DH) using qPCR (region) and immunohistochemistry (IHC) (cell subtype).

Assessing routes of administration: In various aspects, the expression vector is administered via direct sciatic and femoral nerves (SFN) or intra-articular (IA) injections. Biological response achieved using these routes is compared response achieved using intradermal administration. Direct sciatic nerve injections achieve profound analgesia by transduction of lumbar DRG. Sensory innervation of major joints is accessible through direct IA injection. DRG transduction is achieved with this approach, which offers rapid adoption by clinical experts, case of access, and potentially adequate viral infectivity of all disease-affected dermatomes. While direct peripheral nerve blocks with radiofrequency ablation are easily achieved as an alternative using traditional techniques for some major joints (e.g., knee via genicular blocks, shoulder via suprascapular nerve blocks); peripheral nerve blocks for pain relief of other joints are not feasible (e.g., temporomandibular, hip, ankle, elbow, wrist, etc.). Currently, there are very limited options to treat temporomandibular joint disease (TMJ) pain; intra-articular injection of the expression vector described herein represents a transformational therapy for control of symptoms.

All measures are by individuals masked to treatment. Animals are randomly assigned to groups. Assays are conducted at approximately the same time of day. Routine clinical safety assessments are made (e.g., body wt., general appearance, food consumption, pulse, blood pressure, body temperature). The highest achievable dose (PFU) is used for SFN and IA injections and a series of dilutions (up to ten thousand fold) to optimize subsequent parameters for all expression vectors. Eighteen-naïve male C57BL/6J mice/group are used. Analgesic response is monitored every other day until D14 and weekly thereafter until D28 (end-of-life) (analgesia, antihyeralgesia to mechanical and thermal evoked responses; automated voluntary running wheel (wheel distance/time, wheel time, wheel speed; stride). Additionally, clinical safety assessments at baseline and weekly are made in each mouse (e.g., body wt., general appearance, food consumption, blood pressure, body temperature). Restricted neuronal expression is assessed in peripheral nerves, DRG and DH using QPCR (region) and IHC (cell subtype with double staining). Direct DRG transduction may also be achieved using transforaminal epidural injection.

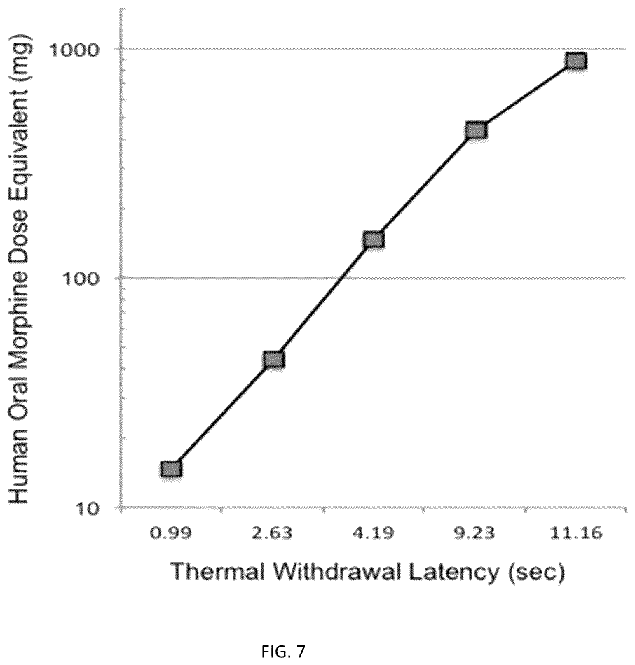

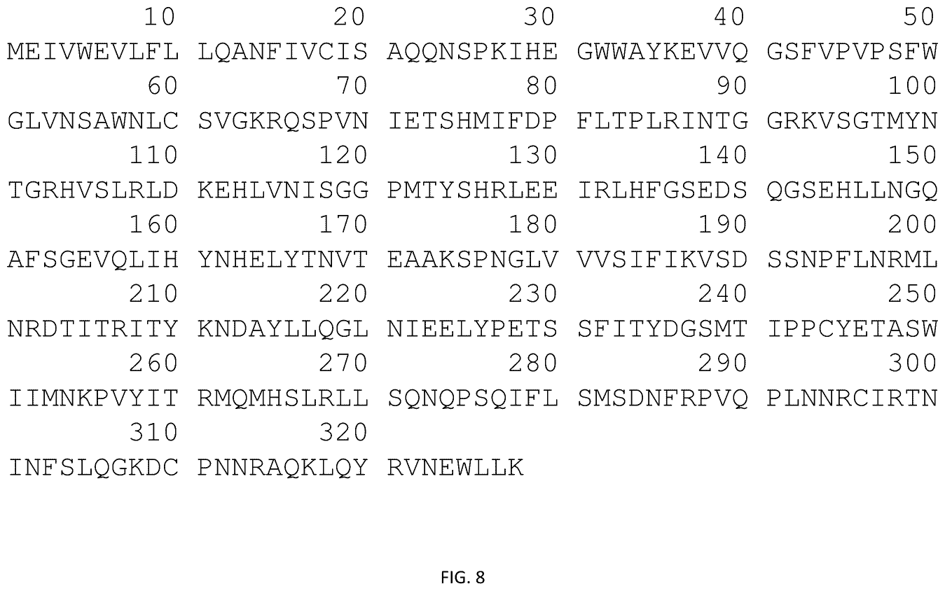

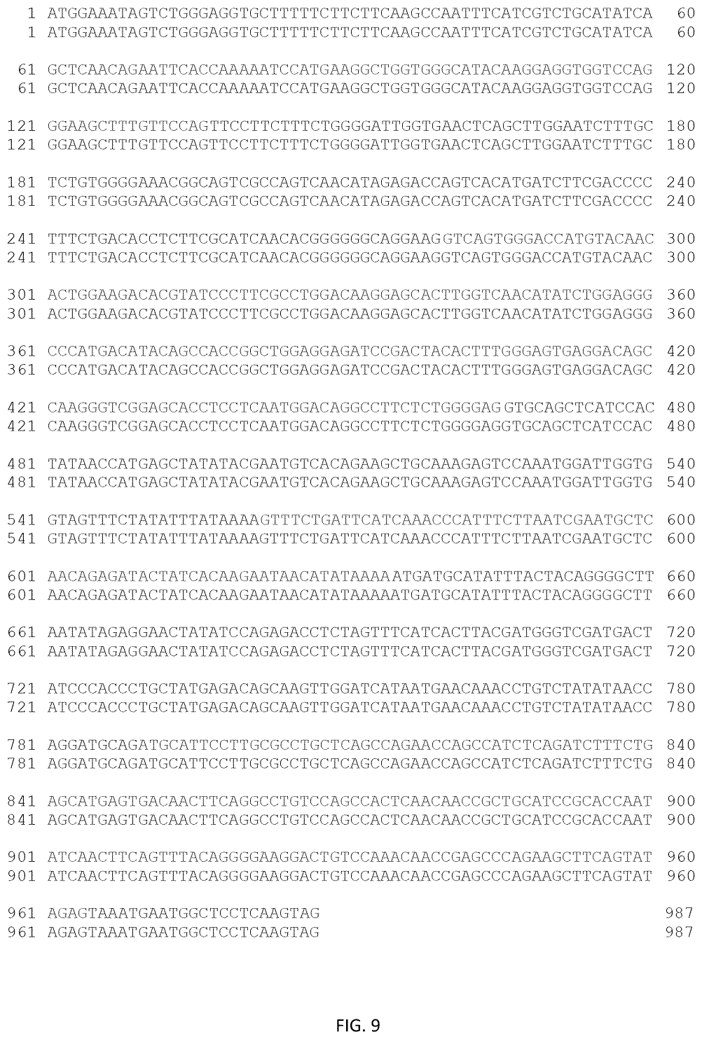

Pharmacodynamics: Bioassays of Analgesia and Anti-hyperalgesia: Both chronic inflammatory and chronic neuropathic pain models are employed. These studies include protection (prevention) trials for CFA (expression vector injected prior to pain) and protection and treatment trials for SNL and OA (expression vector is injected after the nerve injury occurs and assessed over time). Direct sciatic nerve injection prevents and treats chronic neuropathic pain in the SNL model (see FIG. 7). Preliminary short and long-term safety-related-to-mechanism studies and off-target toxicity may be performed in two models. All measures are recorded individuals masked to treatment. Animals are randomly assigned to groups. Assays will be conducted at approximately the same time of day. Clinical pathology, gross inspection, organ weights, and histopathology will be assessed; and CA8, CA8-204, CA10, CA11, ITPR1, pITPR1 measured using DRG, spinal cord, CSF and blood. DRG neuronal and glial apoptosis is examined. In addition to efficacy assessments, clinical safety assessments are made (e.g., body wt., general appearance, food consumption, blood pressure, body temperature). Based on the time course of response, a “treatment” design is employed wherever feasible. The direct nerve “block” approach is potentially applicable to a variety of pain disorders including chronic headache, trigeminal neuralgia, and other craniofacial pain disorders. If desired, the route is altered based on the specific model and potential clinical application. For example, the IA route is particularly relevant to a TMJD. Because this technique is challenging in mice, the knee OA model may be employed as a surrogate because this is both feasible and a chronic prevalent clinical condition. Advanced OA, like TMJD may cause pain at rest (i.e., spontaneous or neuropathic pain) that is generally resistant to non-steroidal anti-inflammatory drugs (NSAIDs), and therefore characterized by both neuropathic and nociceptive pain. The well-established monosodium iodoacetate (MIA) intra-articular injection model of OA that elicits weight-bearing asymmetry due to joint osteolysis, cartilage erosion, and referred tactile and thermal hypersensitivity in mice is useful in this regard. This model was previously shown to produce spontaneous pain unrelieved with diclofenac, TRPVI and TRPAI antagonists, but entirely relieved with intra-articular lidocaine. IA injection of expression vector mediating Nav1.8 specific LALA (Nav being the target of lidocaine and other short-acting local anesthetics) will be efficacious in this model.

Promoters: Promoter sequences useful in the context of the studies described herein include, but are not limited to: TrkB, TrkC, Nav1.9, other Nav gene promoters, NMDA promoter, advillin, CGRP, 5HT, NK1, ASIC3, NPY or NF200 to drive expression of CA expressing sequences including CA8, CA8 fragment (such as CA8-204), CA10, CA11 and the non-human orthologs including Car8, Car10 and Car11.

### Results

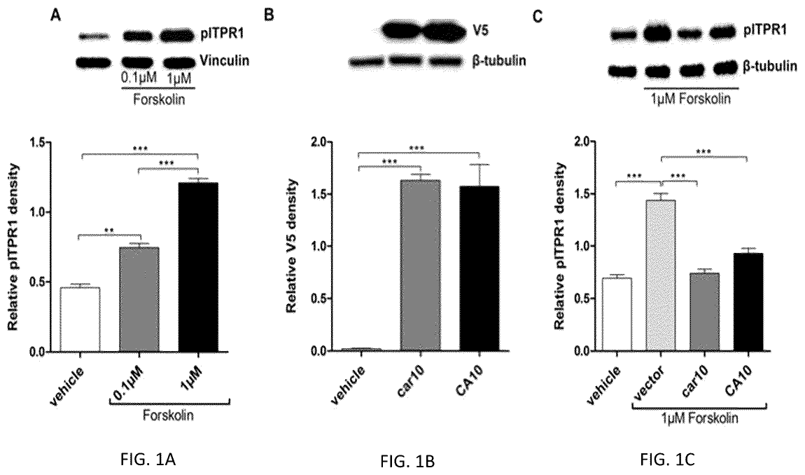

V5-Car10 and V5-CA10 protein overexpression inhibits forskolin-induced pITPR1 in vitro: Results described herein demonstrate, e.g., use of pharmacodynamics bioassays to examine the effects of Car10 on the regulation of ITPR1 activation by phosphorylation (pITPR1) that enhances the response of ITPR1 to the IP3 ligand. HEK293 cells were transfected using AAV8-V5 vectors overexpressing Car10 and CA10, an empty vector and vehicle served as controls. A V5 sequence was inserted at the Car10 or CA10 C-terminal region in order to differentiate between exogenous and endogenous CA10/Car10 expression. Western blot analysis demonstrated that forskolin increases pITPR1 levels in a dose-dependent manner (FIG. 1A). Using the V5 tag, protein overexpression was detected following V5-Car10 and V5-CA10 vector transfection (FIG. 1B). Car10 and CA10 overexpression reduced forskolin-induced ITPR1 phosphorylation in HEK293 cells, whereas empty vector did not alter ITPR1 phosphorylation (FIG. 1C).

Using IHC, increased pITPR1 was observed in HEK293 cells in response to 1 μM forskolin after transfection with empty vector (AAV-null), but after transfection with V5-Car10 and V5-CA10, there was no increase in pITPR1. These data demonstrate that Car10 and CA10 are sufficient to inhibit modulatory domain phosphorylation at Ser-1755 in HEK293 cells, critical to ITPR1 activation and IP3-induced calcium release.

Overexpression of V5-Car10 and V5-CA10 inhibits ATP-induced free calcium release in vitro: ITPR1 contains functionally distinct domains, including the ‘modulatory’ domain responding to intracellular modulators such as calcium, calmodulin, ATP, and carbonic anhydrase-8 (Car8). ATP increases ITPR1-dependent calcium release by increasing the open probability of the channel in the presence of activating concentrations of IP3 and calcium. It is believed that Car8-mediated inhibition of ITPR1 activation and ATP-mediated calcium release requires binding to this modulatory domain. Therefore, the ability of CA8 fragments (e.g., CA8-204), Car10 and CA10 to bind to ITPR1 and pITPR1 was examined. Co-immunoprecipitation of each protein with antibodies to V5 tag with ITPR1 and pITPR1 before and after forskolin stimulation of HEK293 cells was conducted. Western blotting shows that CA8-204, Car10 and CA10 do not bind to ITPR1. Surprisingly, in the functional bioassay evaluating ITPR1 activation (e.g., pITPR1), co-immunoprecipitation of these proteins shows all of these nonbinding proteins inhibit ITPR1 activation, showing a reduction in pITPR1.

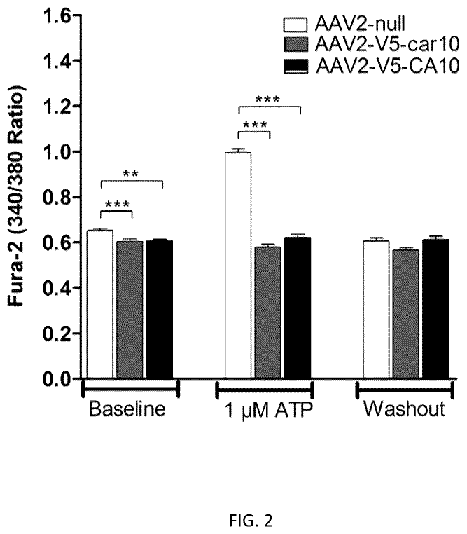

To further examine whether FLAG-CA8-204 (data not shown), V5-Car10 and V5-CA10 overexpression can inhibit ATP-induced calcium release, HEK293 cells were infected with each if these constructs and real-time intracellular calcium concentrations at baseline and in response to ATP stimulation were measured (FIG. 2). ATP stimulated calcium release and an increase in cytosolic free calcium levels in a dose-dependent manner. Cytoplasmic free calcium levels after AAV-null transfection were increased in response to 1 μM ATP compared to baseline in HEK293 cells. In contrast, free calcium concentrations were unchanged in response to 1 μM ATP after transfection with AAV-FLAG-CA8-204, AAV-V5-Car10 and AAV-V5-CA10 and compared to baseline and AAV-V5-CA8. These data demonstrate that CA8-204, Car10 and CA10 can inhibit ITPR1 activation and thereby reduce ATP-stimulated cytoplasmic free calcium levels in these cells.

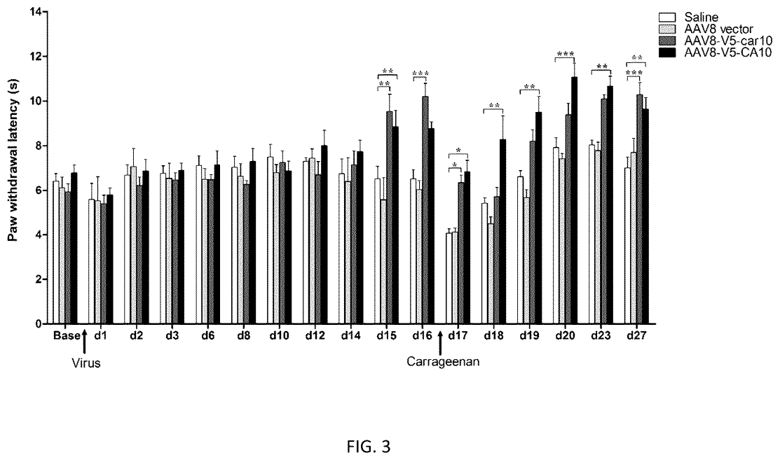

Car10 and CA10 sciatic nerve gene therapy produces analgesia and inhibits inflammatory pain behaviors: Both AAV8-V5-Car10 and AAV8-V5-CA10 produce analgesia (increase in thermal latencies from baseline) after sciatic nerve injections in C57BL/6 mice as compared to controls by Day 15 after injections. Intraplantar injections of carrageenan on Day 16 (after thermal testing) produced acute thermal hypersensitivity in saline and AAV8-null control groups on Days 17 and 18. However, both AAV8-V5-Car10 and AAV8-V5-CA10 showed no hypersensitivity (thermal latencies below baseline) after carrageenan injections on Day 16. Analgesia recurred in both AAV8-V5-Car10 and AAV8-V5-CA10 groups after Day 18, and was maintained through Day 27. No similar finding was observed in the control groups. See FIG. 3.

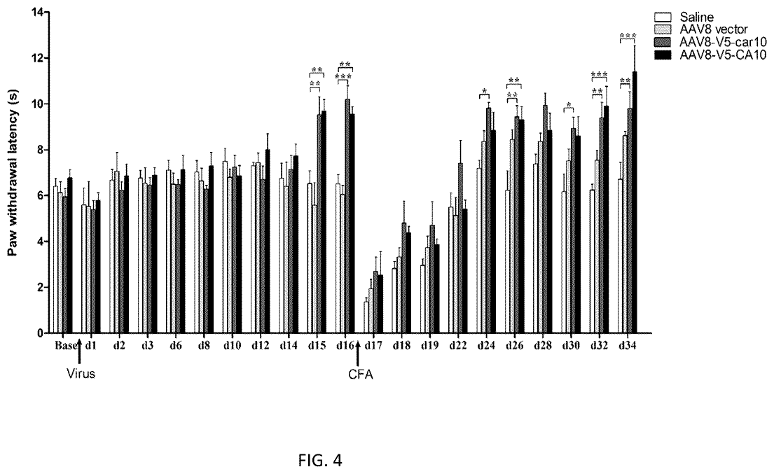

Similarly, both AAV8-V5-Car10 and AAV8-V5-CA10 produce analgesia (increase in thermal latencies from baseline) after sciatic nerve injections in C57BL/6 mice as compared to controls by Day 15 after viral injections in a Complete Freund's adjuvant (CFA) chronic inflammatory pain mouse model. Intraplantar injections of CFA on Day 16 (after thermal testing) produced acute thermal hypersensitivity in all groups on Days 17-19. Both the AAV8-V5-Car10 and AAV8-V5-CA10 groups appeared to recover by Day 24, demonstrating analgesia through Day 34, similar to that observed on Day 16 before CFA injections. See FIG. 4.

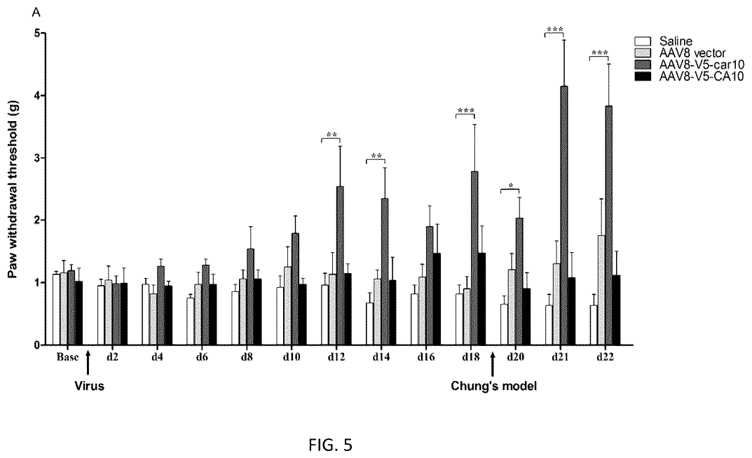

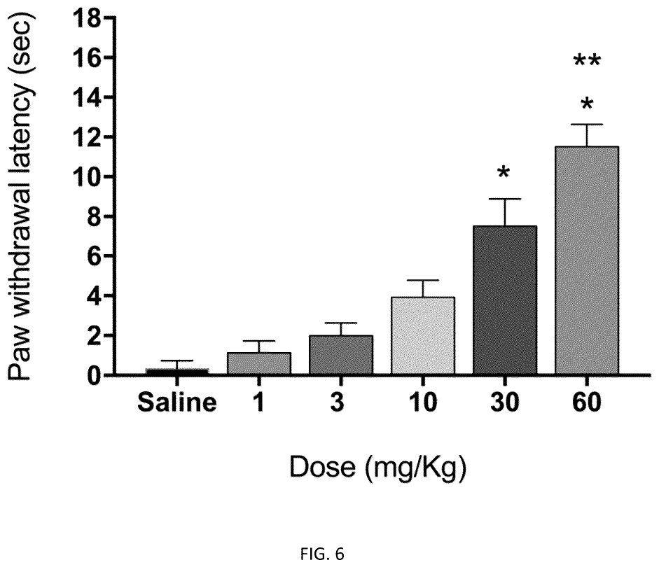

Car10 gene therapy produces analgesia and inhibits neuropathic pain behaviors: The effect of administration of the viral vector of the disclosure in the prevention of neuropathic pain was examined using the Chung mouse model. Chaplan et al., J Neurosci Methods. 1994;53 (1): 55-63. AAV8-V5-Car10 produced analgesic responses in mechanical withdrawal thresholds on Day 12 through Day 22, despite spinal nerve ligation on Day 19. There was no similar increase in withdrawal thresholds in any other group (FIG. 5).

### Discussion

The data described herein demonstrate for the first time that (1) overexpression of a CA8 fragment (CA8-204), CA10, and Car10 proteins in vitro inhibits modulatory domain phosphorylation of ITPR1 at Ser-1755 in response to forskolin; (2) overexpression of a CA8 fragment (CA8-204), CA10, and Car10 inhibits ATP-stimulated intracellular calcium release in vitro; (3) and gene transfer of AAV8-V5-CA10 and AAV8-V5-Car10 to nociceptors via sciatic nerve injections into C57BL/6J mice produces profound analgesia and prevents anti-hyperalgesia using inflammatory and neuropathic pain models. These findings establish for the first time that a CA8 fragment (CA8-204), CA10, and Car10 regulate the ITPR1-cytosolic free calcium-signaling pathway, critical to nociception and pain.

CA10 may participate in other functions including regulation of chondrocytes. Loss of CA10 expression could potentially lead to chondroblastoma formation through dysregulation of the ITPR1-cytosolic free calcium-signaling pathway. Therefore, treatment of chondroblastoma may be derived by overexpression of CA10, CA8, or CA8 fragments using viral vectors.

Osteoarthritis (OA) is characterized by cartilage degradation. Akkiraju et al., J Dev Biol. 2015;3 (4): 177-192. Interestingly, recent genome-wide association between copy number variants (CNV) with OA susceptibility in a Korean population also demonstrated strong association between OA and CA10. Moon et al., BMC Musculoskelet Disord. 2015;16:76. Furthermore, Mori et al., described genetic association between single nucleotide polymorphisms in CA8 and CA10 with spine and femoral bone mineral density (BMD) associated with osteoporosis in Japanese women. Mori et al., J Bone Miner Metab. 2009;27 (2): 213-216. These investigators suggested that genetic variants at the CA8 and CA10 loci might be important determinants of osteoporosis in these and potentially other women. If these relationships hold true in the broader population, it would be reasonable to test whether functional variants at the CA8 and CA10 loci are associated with OA disease severity, pain and disability. Moreover, it seems relevant to test the role of the ITPR1-cytosolic free calcium-signaling pathway and whether functional variants in CA8, CA10, and CA11 may impact ITPR1 mediated calcium release differently in osteoclast and chondrocyte regulation and thereby influence osteoporosis and osteoarthritis differently.

Finally, mental health disorders are frequently comorbid with chronic pain. Trinucleotide repeat expansion is associated with the heritability of fragile-X syndrome, Huntington's disease, myotonic dystrophy and spinocerebellar ataxia. Akkiraju et al., J Dev Biol. 2015;3 (4): 177-192. Additionally, unstable repeats have also been implicated in schizophrenia and bipolar disorder. Vincent et al., Psychiatr Genet. 2016;26 (4): 156-165. Subsequent studies show that much of the signal in psychiatric disease originates from three regions harboring large repeats on chromosome 13921.33, 17q21.33-q22, and 18q21.2. The 17q trinucleotide expansion is located within an intron of the CA10 gene. Vincent et al., supra; Ikeuchi et al., Genomics. 1998;49 (2): 321-326. Given the new potential roles for CA10 described herein, it is worthwhile to revisit the relationship between loss of function due to CA10 functional variants, including this unstable trinucleotide repeat and osteoarthritis, osteoporosis, chronic pain conditions and the use of CA10 overexpression in these affected individuals using compositions and methods described herein to treat these disorders.

In summary, this Example demonstrates the utility of PK/PD bioassays and animal models and routes of administration of viral vectors encoding CA8, CA8 fragment (CA8-204) fragment, and CA10 to treat pain. In particular, these data establish that the materials and methods described herein produce analgesia and inhibit both inflammatory and neuropathic pain.

### Example 2

The following Example demonstrates that administration of an adeno-associated viral vector encoding CA8 fragments described herein (AAV8-FLAG-CA8^{204C} (CA8^{204C}) and the AAV8-FLAG-CA8^{204G} (CA8^{204G}) produces analgesia anti-hyperalgesia in a clinically relevant animal model, the carrageenan inflammatory pain model.

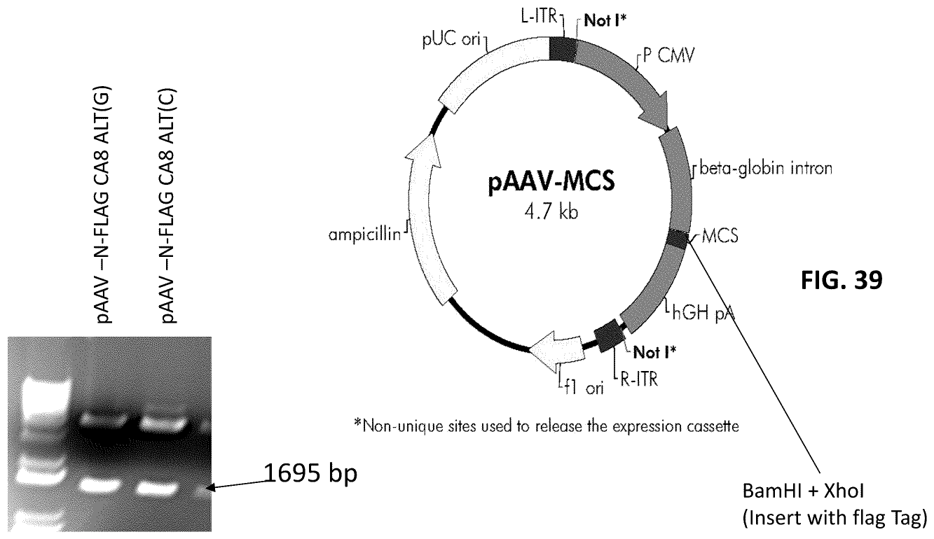

Construction of pAAV-flag-CA8-204^{G} (ALT G) and pAAV-flag-CA8-204^{C} (ALT C). SalI and KpnI sites containing primers were designed for constructing full length alternatively spliced variant (CA8-204) with “G” or “C” at SNP rs6471859. The pAAV-MCS expression construct is shown to the right (vector map on right). See FIG. 39.

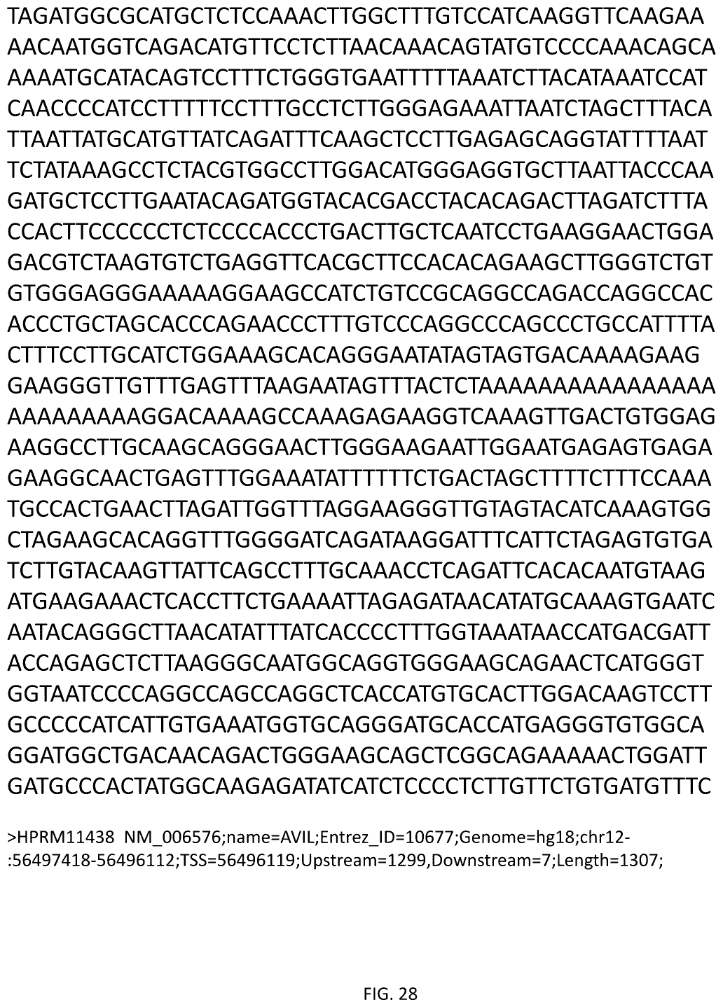

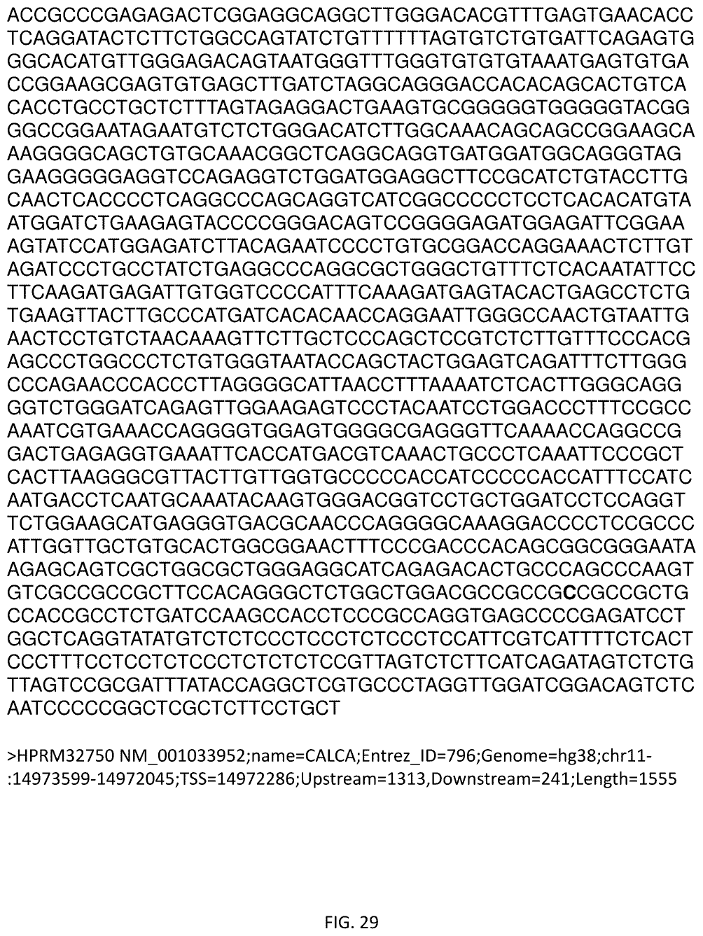

Paw withdrawal thermal latencies were measured at the baseline, and various days following viral vector administration (sciatic nerve injection (SN)) and/or carrageenan injection (left paw). Seven days following administration, mice (n=8 mice per group) that received SN injections of AAV8-FLAG-CA8^{204C} (CA8^{204C}) or AAV8-FLAG-CA8^{204G} (CA8^{204G}) (1.5 μl, 1×10¹³ genome copies/ml) had increasing paw withdrawal latencies, compared to mice administered AAV8-V5-CA8 WT (CA8 WT; positive control) and AAV8-V5-CA8 MT (CA8 mutant; negative control). At day 15 post-viral vector administration, after receiving carrageenan injections, mice in the CA8 MT (negative control) group showed markedly reduced paw withdrawal on days 16 to 18, indicating failure to recuperate from inflammatory pain induced by carrageenan. The mice in both CA8 WT, CA8^{204G} and CA8^{204C} groups, however, demonstrated enhanced paw withdrawal latency, indicating the anti-hyperalgesia protection provided by the CA8 WT, CA8^{204G} and CA8^{204C} (N=8, **** P<0.0001 *** P<0.001, two way Anova statistical group test GraphPad). See FIGS. 31 and 32.

### Example 3

This Example demonstrates that CA10 binds to RYR and pRYR, as demonstrated by co-immunoprecipitation. NBL cells were transfected with AAV-V5-CA10 and AAV-V5-Car10 using Lipofectamin (LTX). Cellular protein was extracted 48h after transfection.

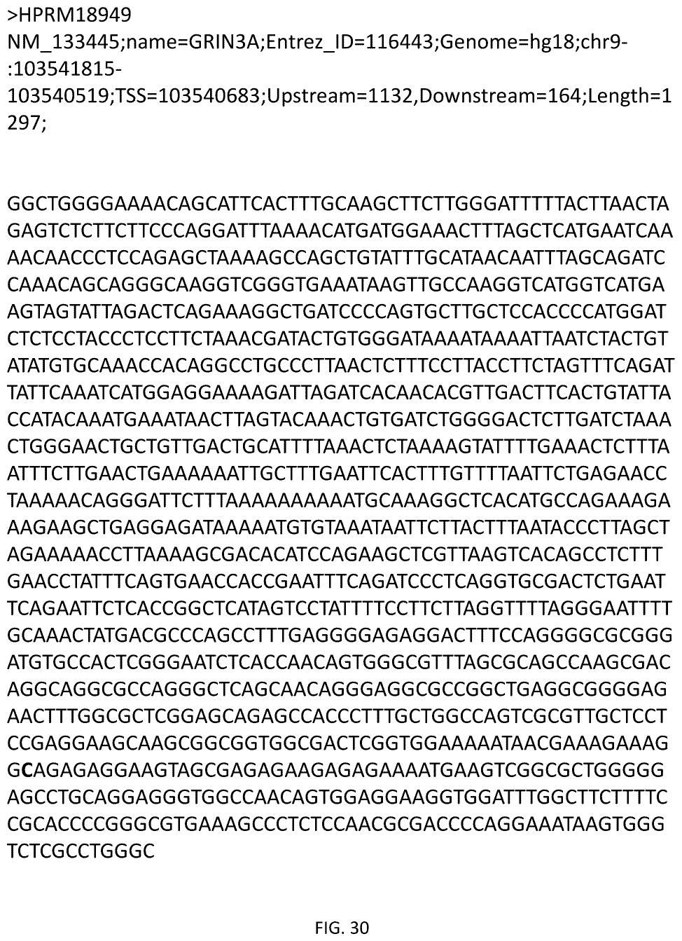

Immunoprecipitation (IP) and western blotting (WB) was utilized to detect binding. Rabbit anti-RYR1 was used for IP and chicken anti-V5 was used for WB analyses. About 1/10 of the IP protein was used for WB to show the V5 labeled CA10 or Car10 by WB. B-actin was used as a loading control. See FIG. 33, which establishes co-immunoprecipitation of CA10 and RYR. Similar results were obtained with CA10 and ITPR1.

These data suggest for the first time that CA10 may also possibly regulate RYR-dependent calcium release through binding to RYR1 and RYR3.

### Example 4

This Example demonstrates that V5-CA10 protein overexpression inhibits forskolin-induced pITPR1 in vitro.

In order to examine the role of CA10 in the regulation of ITPR1 phosphorylation (pITPR1) that enhances the response of ITPR1 to the IP3 ligand, HEK293 cells were transfected with AAV8-V5-CA10 vectors. Transfection with empty vectors or application of vehicle served as controls. A V5 sequence was inserted at the C-terminal region of CA10 in order to differentiate between exogenous and endogenous expression of the native protein in tissues or cell lines. Cells were stained for nuclei with DAPI or pITPR1 and merged.

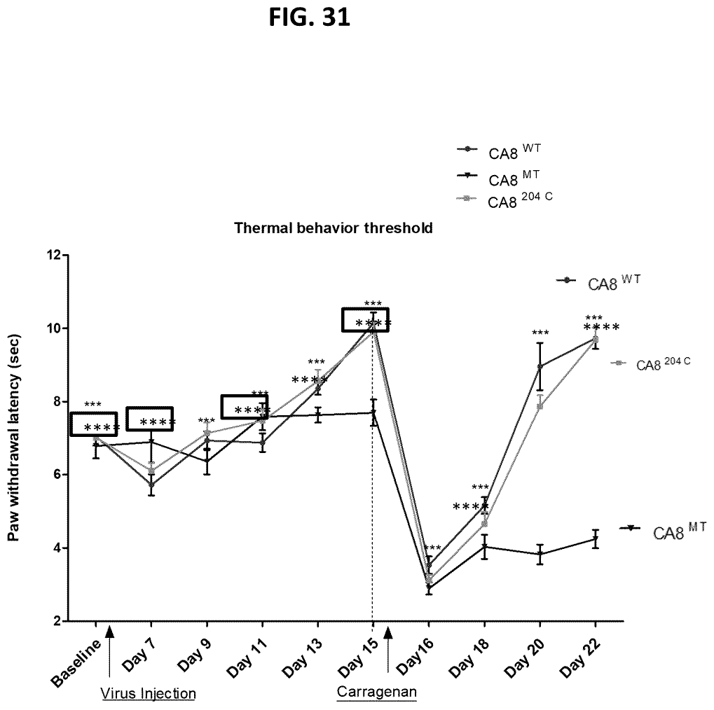

Using the V5 tag, protein overexpression was observed following AAV-V5-CA10 (and murine V5-Car10-encoding vectors) transfections using Western blot analyses (FIG. 34A). CA10 and Car10 overexpression reduced forskolin-induced ITPR1 phosphorylation in HEK293 cells, whereas empty vector did not alter pITPR1 levels (FIG. 34B). These experiments indicate that overexpression of CA10 (and murine Car10) in NBL cells in vitro inhibits modulatory domain phosphorylation of ITPR1 at Ser-1755 in response to forskolin.

### Example 5

The following Example demonstrates that CA10 overexpression inhibits ITPR1-and RYR-mediated calcium release in response to pain mediators. Surprisingly, the studies described herein demonstrate that, in NBL cells, 5HT-mediated RYR-dependent calcium release in nearly completely inhibited by ryanodine. Moreover, V5-CA10 overexpression in NBL cells was observed to also inhibit 5HT-mediated calcium release. Therefore, 5HT-mediated signaling in NBL cells appears to be largely through RYR.

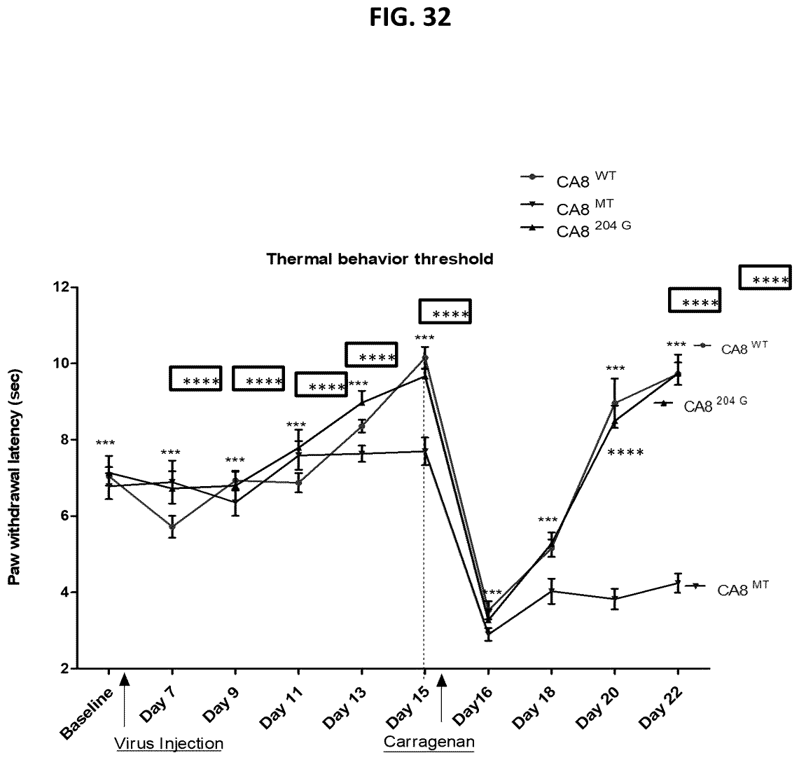

Next, it was determined that overexpression of V5-Car10 and V5-CA10 in HEK293 cells inhibits ATP-induced cytoplasmic calcium release. Fura2 calcium imaging data demonstrated that Car10 and CA10 protein overexpression inhibits ITPR1-mediated cytoplasmic calcium release to 1 μM ATP in HEK293 cells when compared to empty vector control (P<0.001). (N=4 coverslips and a total of 200 cells per sample ** P<0.01, *** P<0.001, by two way ANOVA followed by Bonferroni test.) See FIG. 35.

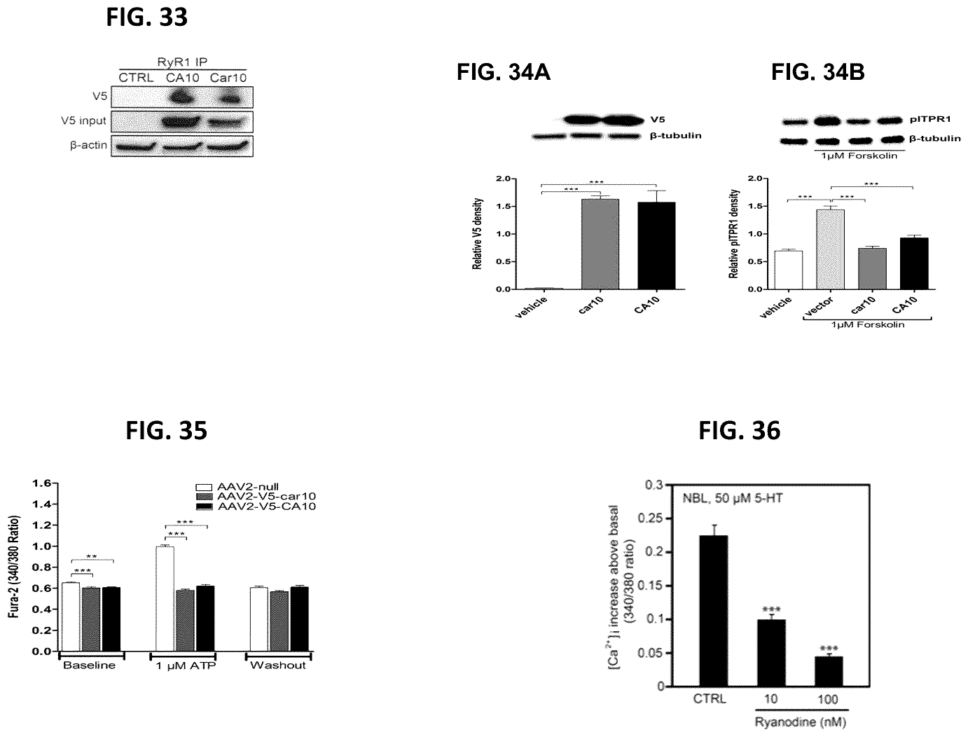

It was also determined that 5HT-induced RYR-dependent calcium release in NBL cells is inhibited by ryanodine. Fura2 calcium imaging data demonstrated that 50 μM 5HT-induced cytoplasmic calcium release in NBL cells was significantly inhibited by ryanodine in a dose-dependent manner, when compared to vehicle control (P<0.001). (N=4 coverslips and a total of 200 cells per sample ** P<0.01, *** P<0.001, by two way ANOVA followed by Bonferroni test). See FIG. 36.

These findings suggest that CA10 regulates the ITPR1-cytosolic free calcium-signaling pathway, critical to nociception and pain behaviors. Additionally, the data showed that 5HT stimulates calcium release that is nearly completely inhibited by ryanodine, suggesting serotonin through RYR and not ITPR1.

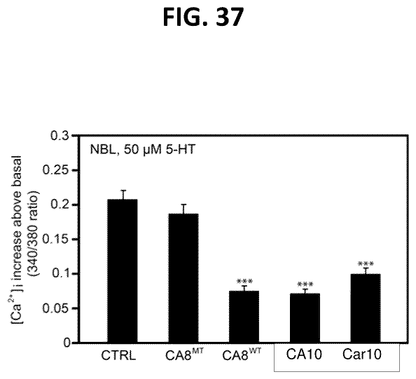

Next, it was determined that overexpression of V5-Car10 and V5-CA10 in NBL cells inhibits 50 μM 5HT-induced cytoplasmic calcium release. See FIG. 37. Fura2 calcium imaging data demonstrated that 50 μM 5HT-induced cytoplasmic calcium release in NBL cells was nearly completely inhibited by 10 nM ryanodine when compared to vehicle control (P<0.001). Additionally, 5HT-induced cytoplasmic calcium release in NBL cells was also inhibited by V5-Car10 and V5-CA10 protein overexpression in NBL cells when compared to empty vector control (P<0.001). (N=4 coverslips and a total of 200 cells per sample ** P<0.01, *** P<0.001, by two way ANOVA followed by Bonferroni test).

### Example 6

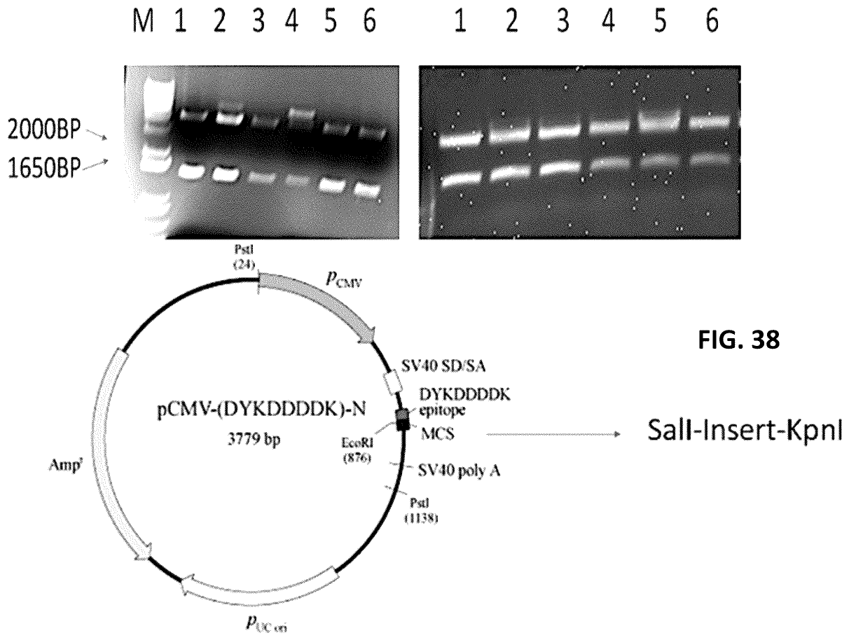

Construction of pCMV-N-flag-CA8-204^{G} and pCMV N-flag-CA8-204^{C}. SalI and KpnI sites containing primers were designed for constructing full length alternative variant (CA8-204) with “G” at rs6471859 SNP, into pCMV-N-flag vector (Clontech). Site directed mutagenesis (Invitrogen) was utilized to construct pCMV-N-flag-CA8-204^{C} producing a novel fragment ending at exon 3 with “C” allele at rs647859. See FIG. 38.

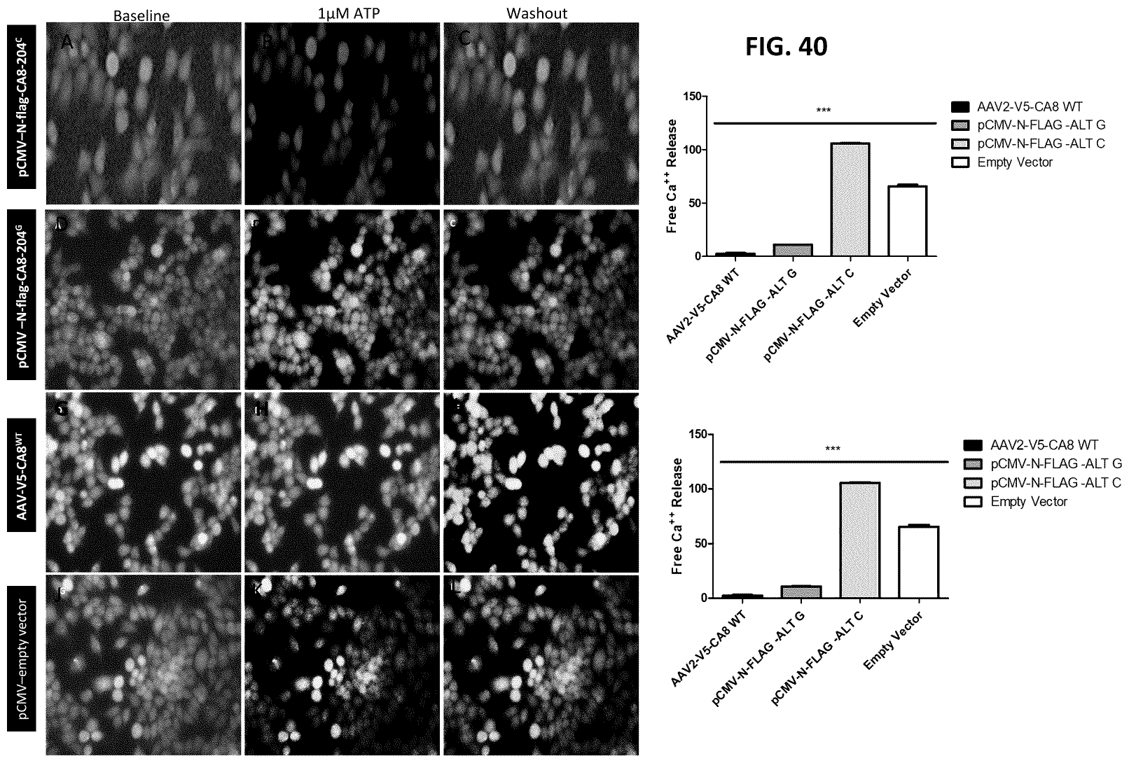

CA8-204^{G} inhibition of calcium release (Ca²⁺ Fura2 imaging) in HEK293 cells. HEK293 cells transfected with pCMV-N-flag-CA8-204G (1,695 bp), pCMV-N-flag-CA8-204^{C}, AAV2-V5-CA8^{WT} (positive control), or empty vector (negative control). (n=6,number of coverslips in each experiment, number of experiments=3, P-value <0.001, comparison between three groups was carried out by Tukey's multiple comparison test (GraphPad Prism Software). See FIG. 40.

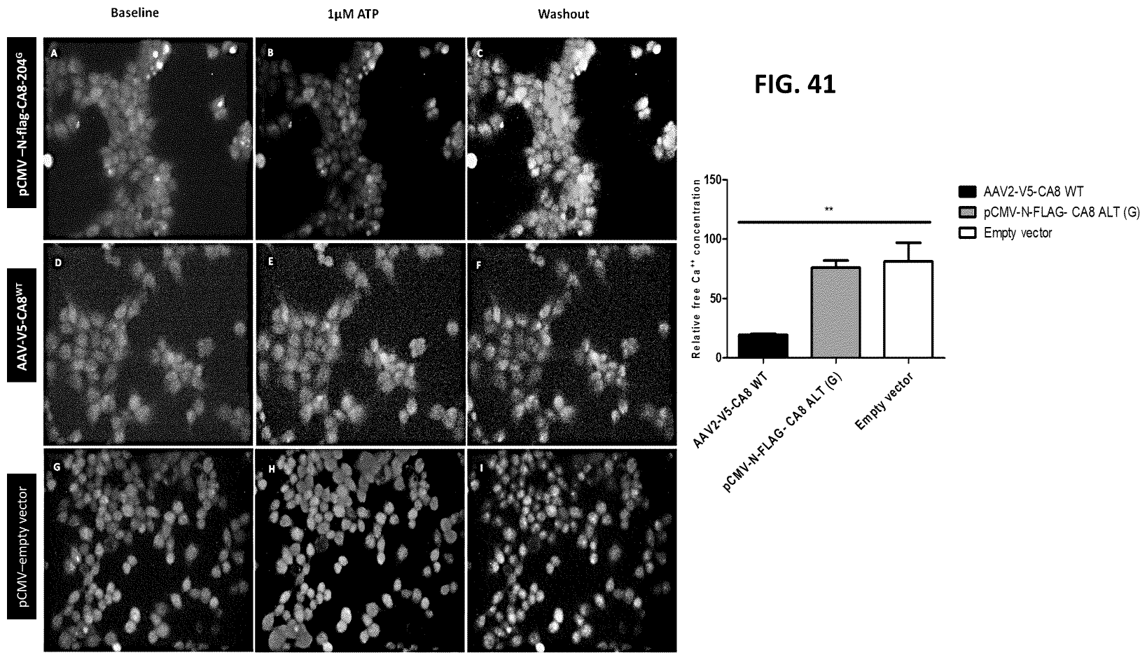

Differential tissue expression of CA8 ALT (G) and CA8 ALT (C) in HEK293 and NBL cells. Quantitative RT-PCR (Real Time PCR, Applied Biosystems) from (a) HEK293 cells and (b) Neuronal cells (NBL) after transfections with alternative variant pCMV-FLAG-N CA8 ALTG with “G” allele at rs6471859 (bp 1417 of CA8-204) or pCMV-FLAG-N CA8 ALTC (“C” allele at rs 6471859). Quantities of respective vectors were normalized using beta Actin (ACTB) gene product. The primers used were designed covering the 3′UTR ewgion of CA8-204. The HK cells do not express detectable CA8-002 transcript with the C genotype at variant rs6471859. N=3, number of experiments=3 (Statistical analysis was done using GraphPad Prism Software, **=p-Value <0.001, ***=p-Value <0.0001, One Way Anova was applied followed by Tukey group comparison. See FIGS. 41A and 41B.

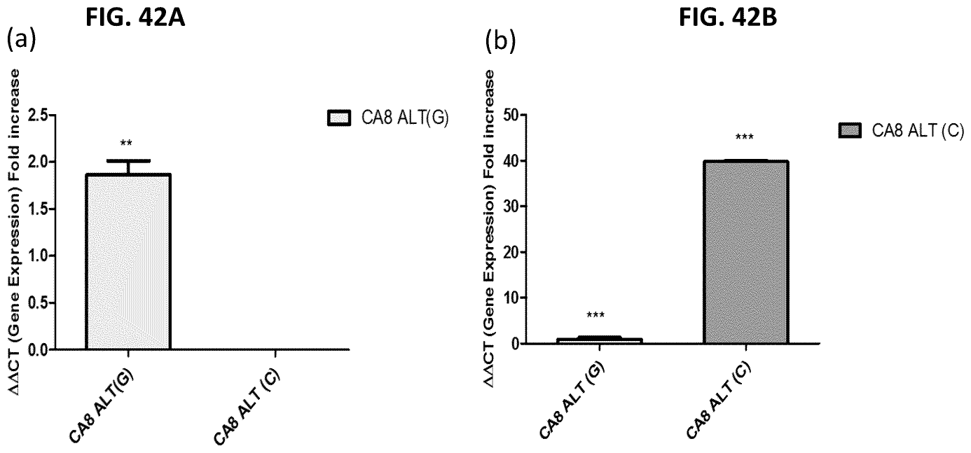

CA8-204^{C} and CA8-204^{G} fragments show variable tissue expression. HEK 293 (non-neuronal cells) and NBL (neuronal cells) were transfected with vectors expressing CA8-204€ and CA8-204G transcripts and subjected to quantitative RT-PCR (QPCR-Applied Biosystems) using SyBr green (AB), normalized using beta-actin as an internal control. The primers were designed to flank the 3′UTR region of the CA8-204^{C} and CA8-204^{G} sequences expressed selectively in these cell lines. We observe a marked difference of CA8 fragment splicing and expression where the CA8-204^{C} is expressed predominantly in NBL cells and the CA8-204^{G} is expressed predominantly in the HEK293 cells consistent with cell specific splicing factors dictating expression of each fragment. (N=4, *** P-Value <0.001, one way ANOVA was applied followed by post-hoc Tukey's test). See FIG. 42.

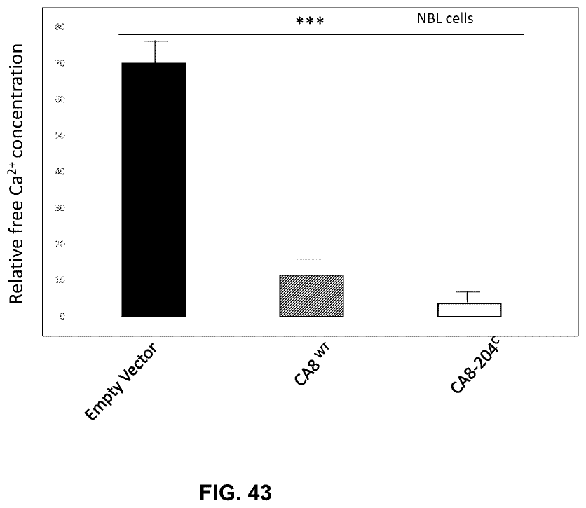

CA8-204^{C} fragment inhibits ATP stimulated calcium release in NBL cells. NBL cells were transfected with vectors with nucleotide inserts expressing CA8-204^{C}, CA8-201 (CA8 wildtype positive control) or empty vector (negative control) and were subjected to calcium imaging (Fura2, Leica Micro Systems), using 1 μM ATP as a stimulant for intracellular calcium release. CA8-204^{C} and CA8-201 were able to inhibit Ca²⁺ release in NBL cells in vitro. Inhibition of calcium release by CA8-204^{C} exceeded that of CA8-201 (wildtype full length transcript/peptide). In contrast, empty vector (negative control) failed to inhibit calcium release in this assay. (N=6, *** P<0.001, statistical analysis led by one way ANOVA, group comparison through Tukcy's post-hoc test, GraphPad Prism). See FIG. 43.

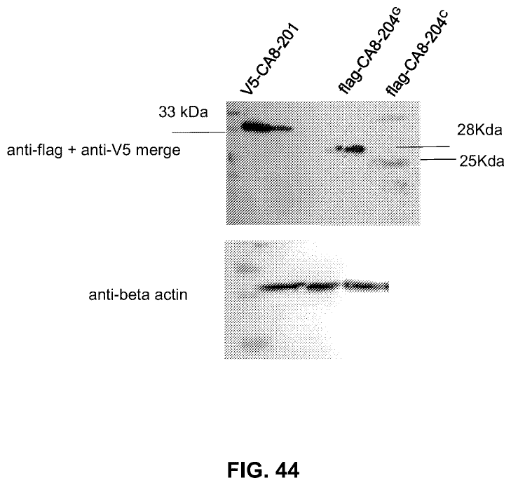

CA8-204^{G} or CA8-204^{C} peptide fragments are 28 or 26 kDa as expressed selectively in HEK293 or NBL cells. Proteins were extracted from HEK293 (middle) or NBL cells (right) transfected with vectors expressing either flag-CA8-204^{G}, flag-CA8-204^{C}, or V5-CA8-201 (wildtype full length CA8) transcripts, run on the same gel and immunoblotted with either anti-flag or anti-V5 antibodies. Only flag-CA8-204^{G} peptide fragment was detected at 28 kDa in HEK293 cells and only flag-CA8-204^{C} peptide fragment at 26kDA was detected in NBL cells. Data was merged after immunoblotting. Data was normalized with beta actin (control). See FIG. 44.

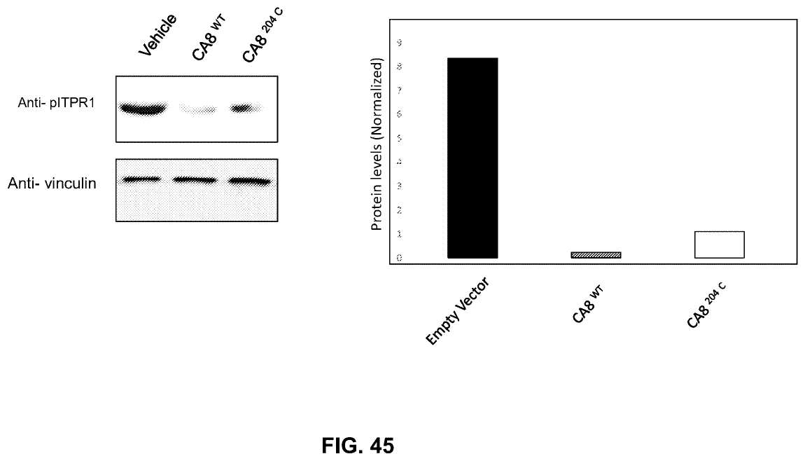

CA8 ^{204 C} inhibits forskolin induced phosphorylation of pITPR1. HEK 293 cells were transfected with vectors containing CA8 ^{WT}, CA8 ^{204 C} or empty vectors (vehicle). Vehicle was used as a negative control. Data was normalized with vinculin as internal control. See FIG. 45.

All publications, patents and patent applications cited in this specification are herein incorporated by reference as if each individual publication or patent application were specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

1. A method of treating or preventing pain in a subject in need thereof, the method comprising administering to the subject an expression vector comprising a nucleic acid sequence encoding carbonic anhydrase 10 or carbonic anhydrase 11 such that the nucleic acid is expressed to produce carbonic anhydrase 10 or carbonic anhydrase 11.

2-17. (Canceled)

18. A method of treating or preventing pain in a subject in need thereof, the method comprising administering to the subject an expression vector comprising a nucleic acid sequence encoding human carbonic anhydrase 8 such that the nucleic acid is expressed to produce human carbonic anhydrase 8.

19. The method of claim 18, wherein the subject is a human.

20. The method of claim 18, wherein the expression vector is a viral vector.

21. The method of claim 20, wherein the viral vector is an adeno-associated viral vector.

22. The method of claim 20, wherein the viral vector is a herpes simplex viral vector.

23. The method of claim 18, wherein the nucleic acid sequence is operably linked to a promoter selected from the group consisting of CMV promoter, TrkA promoter, TrkB promoter, TrkC promoter, Nav1.9 promoter, Nav1.7 promoter, Nav1.8 promoter, NMDA promoter, advillin promoter, CGRP promoter, 5HT promoter, NK1 promoter, ASIC3 promoter, NPY promoter, and NF200 promoter.

24. The method of claim 18, wherein the pain is neuropathic pain.

25. The method of claim 18, wherein the pain is inflammatory pain.

26. The method of claim 18, wherein the pain is caused by cancer or spinal cord injury.

27. The method of claim 18, comprising administering the expression vector to the dorsal root ganglion of the subject.

28. The method of claim 18, comprising administering the expression vector via intra-articular injection.

29. The method of claim 18, comprising administering the expression vector orally.

30. The method of claim 18, comprising administering the expression vector to the trigeminal ganglia.

31. The method of claim 18, comprising administering the expression vector via peripheral nerve injection.

32. The method of claim 18, comprising administering the expression vector via catheter to a site where pain arises.

33. The method of claim 18, comprising administering the expression vector via needle to a site where pain arises.

34. The method of claim 18, comprising use of imaging to administer the expression vector.

35-37. (canceled)

38. The method of claim 18, comprising administering the expression vector by epidural injection to the subject.

39. The method of claim 18, wherein the pain is nociceptive pain.


SEQUENCE LISTING (35 sequences)

SEQ ID NO: 1 - DNA, 455 bp, Homo sapiens
  source 1..455: mol_type = other DNA
tgctgccgagagcgccgaggggccccgcggccttcccatggcggacctgagcttcatcgaagataccgtcgccttccccgagaaggaagaggatgaggaggaagaagaggagggtgtggagtggggctacgaggaaggtgttgagtggggtctggtgtttcctgatgctaatggggaataccagtctcctattaacctaaactcaagagaggctaggtatgacccctcgctgttggatgtccgcctctccccaaattatgtggtgtgccgagactgtgaagtcaccaatgatggacataccattcaggttatcctgaagtcaaaatcagttctttcgggaggaccattgcctcaagggcatgaatttgaactgtacgaagtgagatttcactggggaagagaaaaccagcgtggttctgagcacacggttaatttcaaagcttttcccatggagg

SEQ ID NO: 2 - DNA, 594 bp, Homo sapiens
  source 1..594: mol_type = other DNA
aacgcacgcctgcttgcactcacactgcggttcacacccgcaggcgctctcgcactcacactgccgctccgcgtgctcacactcccccacgcgcgctccgctccggctccagccccgcgcccagcgaaggcgcaggcactgctgccgagagcgccgaggggccccgcggccttcccatggcggacctgagcttcatcgaagataccgtcgccttccccgagaaggaagaggatgaggaggaagaagaggagggtgtggagtggggctacgaggaaggtgttgagtggggtctggtgtttcctgatgctaatggggaataccagtctcctattaacctaaactcaagagaggctaggtatgacccctcgctgttggatgtccgcctctccccaaattatgtggtgtgccgagactgtgaagtcaccaatgatggacataccattcaggttatcctgaagtcaaaatcagttctttcgggaggaccattgcctcaagggcatgaatttgaactgtacgaagtgagatttcactggggaagagaaaaccagcgtggttctgagcacacggttaatttcaaagcttttcccatggagg

SEQ ID NO: 3 - DNA, 3260 bp, Homo sapiens
  source 1..3260: mol_type = other DNA
ctctctgagttgggaagcgtagggatccgtaggcgaggaaataacgacccctgcagttgtattgcggaaaatctcgacagcggcgctagttgcgggcgatggaagccaggcaactgggggttctggggagttcaggaaaatagcagaggagcaggaagggcgcgcgcgacctggagagtctgtgtgcccccaccgcgccccagtccccggggcccagcccttcccctcggcgccctggacgcactgccggaacccggctgagaggctgcaggctgcgcgcggacctggggagcagggagggtcggcggaggctgccggcggctggcggtttcgggcaataatccctgcctctctttctctgtgtgtctgctgtgtctgctccttccccgccccccggaagcaggagaagaactgccccggagcgcagcagccaccctccgaccatgccccggtgaggggggcggacttcgagggcaacttgccgcggactgcctgggcttagccagcgagctacgcgctcccgggagcccggaattgcacggcgcagcccggcggggggctatcgtctatgtcttcttggggcgccagacgaatcggggtctcgtttttgctggaagagcccagtgttggtggcttcaggtggctgctgccgccgccgccgccgccgccgctgctagtgcggtttccgccgctggtgcgaagagaagagacacgcgagcggggagacctccaaggcagcgaggcatcggacatgtgtcagcacatctggggcgcacatccgtcgagcccgaggggagatttgccggaacaattcaaactgcgatattgatcttgggggtgactgtccctggccggctgtcgggtgggagtgcgagtgtgcactcgctcggaagtgtgtgcgagtgtgtatgtgtgtgtgccgtgtcgggctccccccttccccccgttttcccgtcgagtgatgcacttggaatgagaatcagaggatggaaatagtctgggaggtgctttttcttcttcaagccaatttcatcgtctgcatatcagctcaacagaattcaccaaaaatccatgaaggctggtgggcatacaaggaggtggtccagggaagctttgttccagttccttctttctggggattggtgaactcagcttggaatctttgctctgtggggaaacggcagtcgccagtcaacatagagaccagtcacatgatcttcgacccctttctgacacctcttcgcatcaacacggggggcaggaaggtcagtgggaccatgtacaacactggaagacacgtatcccttcgcctggacaaggagcacttggtcaacatatctggagggcccatgacatacagccaccggctggaggagatccgactacactttgggagtgaggacagccaagggtcggagcacctcctcaatggacaggccttctctggggaggtgcagctcatccactataaccatgagctatatacgaatgtcacagaagctgcaaagagtccaaatggattggtggtagtttctatatttataaaagtttctgattcatcaaacccatttcttaatcgaatgctcaacagagatactatcacaagaataacatataaaaatgatgcatatttactacaggggcttaatatagaggaactatatccagagacctctagtttcatcacttacgatgggtcgatgactatcccaccctgctatgagacagcaagttggatcataatgaacaaacctgtctatataaccaggatgcagatgcattccttgcgcctgctcagccagaaccagccatctcagatctttctgagcatgagtgacaacttcaggcctgtccagccactcaacaaccgctgcatccgcaccaatatcaacttcagtttacaggggaaggactgtccaaacaaccgagcccagaagcttcagtatagagtaaatgaatggctcctcaagtagggaacaaagccaagaagaatcccacctcagtgaaatgctacaactgtgaattgacgtaacctagaatgtcccccttcttgcttctctctccttctttcccccaagcctcattcattcttgggattggccctttcttcatgaaaagtgtctgcaaaaccatggcagaggaatacatctctcacacatactcacaaacacacacacaagcacttgcacatacatacaaacacatgcaaacatacctacacacacacacactcttacaacctccatcatgggaagtcaagtttcagaaacaaaagtctcattcataagaggtcttagaagaaaataaccagttaacctgatttcaattttgataccgttttcctgaactaataaatctacccaatgagacttttcagcctttgtacatacaaaattcttccaaaagagagaggagaaaatacagctctgatggcatcaaacggactttgcatcaagtaatttcagatagtgtcctaggatcctttgagggtgctggtagcaggtgagcaggacaaagttgaccaaggacacttatttctagattatgattcttctgtttactcaacaatttacaaagaaaaaaaggacagacattgaagagctacacattgtatatatatcaccacagactataaggaaatggaattatttccctctttgtcacatatctgtagtaggatttgccaagatcagaaatgatccatttgctgtttcttgttttccaaaggtcatacattgtgtttggttattgttaccagctcaataaatgtgtttaacgagttaatttcatttttctggctttggtctgttctccttccttacaggctaagccctggctccatgcaactgcattctttgatttcacttgttccttcatctacatgttttgttcatttgcagccagtttttactgagtttgtggcaatcaggaatgcatttgctaagcaagtatgactttaattccactccatggctcaatcattcacatgaggtgagcttcagcctgagatagcaggcgacagacttcttgcgtttcaaaactgccatgcccccctgtgatgctcccgtgaaggaatgcactttgccttgtaagttcctgggaaaggggtatgttttctctccaggtgcagccagatctcacaaagtacaaaacgaatgcctttcttttcttgtttataatggtcactcactgtgtttggttactgtcaagaaatcaataaatgtgtttaacaagttacccagtaaaaaaaaaaaaaaaaa

SEQ ID NO: 4 - protein, 328 aa, Homo sapiens
MEIVWEVLFLLQANFIVCISAQQNSPKIHEGWWAYKEVVQGSFVPVPSFWGLVNSAWNLCSVGKRQSPVNIETSHMIFDPFLTPLRINTGGRKVSGTMYNTGRHVSLRLDKEHLVNISGGPMTYSHRLEEIRLHFGSEDSQGSEHLLNGQAFSGEVQLIHYNHELYTNVTEAAKSPNGLVVVSIFIKVSDSSNPFLNRMLNRDTITRITYKNDAYLLQGLNIEELYPETSSFITYDGSMTIPPCYETASWIIMNKPVYITRMQMHSLRLLSQNQPSQIFLSMSDNFRPVQPLNNRCIRTNINFSLQGKDCPNNRAQKLQYRVNEWLLK

SEQ ID NO: 5 - DNA, 2951 bp, Homo sapiens
  source 1..2951: mol_type = other DNA
acagttcccaatcgggacaagcgcgccgagaaatcctggatcttcccagggattccctctacgctccctcctcttcttcaataaccccaaagaggagaagaactgccccggagcgcagcagccaccctccgaccatgccccggtgaggggggcggacttcgagggcaacttgccgcggactgcctgggcttagccagcgagctacgcgctcccgggagcccggaattgcacggcgcagcccggcggggggctatcgtctatgtcttcttggggcgccagacgaatcggggtctcgtttttgctggaagagcccagtgttggtggcttcaggtggctgctgccgccgccgccgccgccgccgctgctagtgcggtttccgccgctggtgcgaagagaagagacacgcgagcggggagacctccaaggcagcgaggcatcggacatgtgtcagcacatctggggcgcacatccgtcgagcccgaggggagatttgccggaacaattcaaactgcgatattgatcttgggggtgactgtccctggccggctgtcgggtgggagtgcgagtgtgcactcgctcggaagtgtgtgcgagtgtgtatgtgtgtgtgccgtgtcgggctccccccttccccccgttttcccgtcgagtgatgcacttggaatgagaatcagaggatggaaatagtctgggaggtgctttttcttcttcaagccaatttcatcgtctgcatatcagctcaacagaattcaccaaaaatccatgaaggctggtgggcatacaaggaggtggtccagggaagctttgttccagttccttctttctggggattggtgaactcagcttggaatctttgctctgtggggaaacggcagtcgccagtcaacatagagaccagtcacatgatcttcgacccctttctgacacctcttcgcatcaacacggggggcaggaaggtcagtgggaccatgtacaacactggaagacacgtatcccttcgcctggacaaggagcacttggtcaacatatctggagggcccatgacatacagccaccggctggaggagatccgactacactttgggagtgaggacagccaagggtcggagcacctcctcaatggacaggccttctctggggaggtgcagctcatccactataaccatgagctatatacgaatgtcacagaagctgcaaagagtccaaatggattggtggtagtttctatatttataaaagtttctgattcatcaaacccatttcttaatcgaatgctcaacagagatactatcacaagaataacatataaaaatgatgcatatttactacaggggcttaatatagaggaactatatccagagacctctagtttcatcacttacgatgggtcgatgactatcccaccctgctatgagacagcaagttggatcataatgaacaaacctgtctatataaccaggatgcagatgcattccttgcgcctgctcagccagaaccagccatctcagatctttctgagcatgagtgacaacttcaggcctgtccagccactcaacaaccgctgcatccgcaccaatatcaacttcagtttacaggggaaggactgtccaaacaaccgagcccagaagcttcagtatagagtaaatgaatggctcctcaagtagggaacaaagccaagaagaatcccacctcagtgaaatgctacaactgtgaattgacgtaacctagaatgtcccccttcttgcttctctctccttctttcccccaagcctcattcattcttgggattggccctttcttcatgaaaagtgtctgcaaaaccatggcagaggaatacatctctcacacatactcacaaacacacacacaagcacttgcacatacatacaaacacatgcaaacatacctacacacacacacactcttacaacctccatcatgggaagtcaagtttcagaaacaaaagtctcattcataagaggtcttagaagaaaataaccagttaacctgatttcaattttgataccgttttcctgaactaataaatctacccaatgagacttttcagcctttgtacatacaaaattcttccaaaagagagaggagaaaatacagctctgatggcatcaaacggactttgcatcaagtaatttcagatagtgtcctaggatcctttgagggtgctggtagcaggtgagcaggacaaagttgaccaaggacacttatttctagattatgattcttctgtttactcaacaatttacaaagaaaaaaaggacagacattgaagagctacacattgtatatatatcaccacagactataaggaaatggaattatttccctctttgtcacatatctgtagtaggatttgccaagatcagaaatgatccatttgctgtttcttgttttccaaaggtcatacattgtgtttggttattgttaccagctcaataaatgtgtttaacgagttaatttcatttttctggctttggtctgttctccttccttacaggctaagccctggctccatgcaactgcattctttgatttcacttgttccttcatctacatgttttgttcatttgcagccagtttttactgagtttgtggcaatcaggaatgcatttgctaagcaagtatgactttaattccactccatggctcaatcattcacatgaggtgagcttcagcctgagatagcaggcgacagacttcttgcgtttcaaaactgccatgcccccctgtgatgctcccgtgaaggaatgcactttgccttgtaagttcctgggaaaggggtatgttttctctccaggtgcagccagatctcacaaagtacaaaacgaatgcctttcttttcttgtttataatggtcactcactgtgtttggttactgtcaagaaatcaataaatgtgtttaacaagttacccagtaaaaaaaaaaaaaaaaa

SEQ ID NO: 6 - protein, 328 aa, Homo sapiens; identical to SEQ ID NO: 4

SEQ ID NO: 7 - DNA, 3386 bp, Homo sapiens
  source 1..3386: mol_type = other DNA
ttcaattcccctgcatccacactgcaaccgggtgcaagtctttcctaggttatagacagcccgcctccccacgagaccttctgcggactggctgcccctggcctcggcctgggtgggtgggtggtgctcgcgccgcccctactttcccctggggccctggagagccgcgcgctgggtgtgtgtgtgtgtgcgcgcgcgggtttggtggggaaatctccttcagtggaagctgctgagtgtcgttggcggaaagtatcctggagcccagcaaaagcctccaaagcactagagcttccctaaagaaaagcagaataaaagaaagacaagaagggaagcccgttcctctaaagcccgggtttccaatagtcccggatcctcctggagttgggcgaaggcgcagttggcagcagccgctgctggcacccggacttggaaaggcggtgttcccccttttcgggaggagggaggcagggacttgcaggcaagagttgcacctggtctaggaacctgcagagaaaagaactctggggagaagaactgccccggagcgcagcagccaccctccgaccatgccccggtgaggggggcggacttcgagggcaacttgccgcggactgcctgggcttagccagcgagctacgcgctcccgggagcccggaattgcacggcgcagcccggcggggggctatcgtctatgtcttcttggggcgccagacgaatcggggtctcgtttttgctggaagagcccagtgttggtggcttcaggtggctgctgccgccgccgccgccgccgccgctgctagtgcggtttccgccgctggtgcgaagagaagagacacgcgagcggggagacctccaaggcagcgaggcatcggacatgtgtcagcacatctggggcgcacatccgtcgagcccgaggggagatttgccggaacaattcaaactgcgatattgatcttgggggtgactgtccctggccggctgtcgggtgggagtgcgagtgtgcactcgctcggaagtgtgtgcgagtgtgtatgtgtgtgtgccgtgtcgggctccccccttccccccgttttcccgtcgagtgatgcacttggaatgagaatcagaggatggaaatagtctgggaggtgctttttcttcttcaagccaatttcatcgtctgcatatcagctcaacagaattcaccaaaaatccatgaaggctggtgggcatacaaggaggtggtccagggaagctttgttccagttccttctttctggggattggtgaactcagcttggaatctttgctctgtggggaaacggcagtcgccagtcaacatagagaccagtcacatgatcttcgacccctttctgacacctcttcgcatcaacacggggggcaggaaggtcagtgggaccatgtacaacactggaagacacgtatcccttcgcctggacaaggagcacttggtcaacatatctggagggcccatgacatacagccaccggctggaggagatccgactacactttgggagtgaggacagccaagggtcggagcacctcctcaatggacaggccttctctggggaggtgcagctcatccactataaccatgagctatatacgaatgtcacagaagctgcaaagagtccaaatggattggtggtagtttctatatttataaaagtttctgattcatcaaacccatttcttaatcgaatgctcaacagagatactatcacaagaataacatataaaaatgatgcatatttactacaggggcttaatatagaggaactatatccagagacctctagtttcatcacttacgatgggtcgatgactatcccaccctgctatgagacagcaagttggatcataatgaacaaacctgtctatataaccaggatgcagatgcattccttgcgcctgctcagccagaaccagccatctcagatctttctgagcatgagtgacaacttcaggcctgtccagccactcaacaaccgctgcatccgcaccaatatcaacttcagtttacaggggaaggactgtccaaacaaccgagcccagaagcttcagtatagagtaaatgaatggctcctcaagtagggaacaaagccaagaagaatcccacctcagtgaaatgctacaactgtgaattgacgtaacctagaatgtcccccttcttgcttctctctccttctttcccccaagcctcattcattcttgggattggccctttcttcatgaaaagtgtctgcaaaaccatggcagaggaatacatctctcacacatactcacaaacacacacacaagcacttgcacatacatacaaacacatgcaaacatacctacacacacacacactcttacaacctccatcatgggaagtcaagtttcagaaacaaaagtctcattcataagaggtcttagaagaaaataaccagttaacctgatttcaattttgataccgttttcctgaactaataaatctacccaatgagacttttcagcctttgtacatacaaaattcttccaaaagagagaggagaaaatacagctctgatggcatcaaacggactttgcatcaagtaatttcagatagtgtcctaggatcctttgagggtgctggtagcaggtgagcaggacaaagttgaccaaggacacttatttctagattatgattcttctgtttactcaacaatttacaaagaaaaaaaggacagacattgaagagctacacattgtatatatatcaccacagactataaggaaatggaattatttccctctttgtcacatatctgtagtaggatttgccaagatcagaaatgatccatttgctgtttcttgttttccaaaggtcatacattgtgtttggttattgttaccagctcaataaatgtgtttaacgagttaatttcatttttctggctttggtctgttctccttccttacaggctaagccctggctccatgcaactgcattctttgatttcacttgttccttcatctacatgttttgttcatttgcagccagtttttactgagtttgtggcaatcaggaatgcatttgctaagcaagtatgactttaattccactccatggctcaatcattcacatgaggtgagcttcagcctgagatagcaggcgacagacttcttgcgtttcaaaactgccatgcccccctgtgatgctcccgtgaaggaatgcactttgccttgtaagttcctgggaaaggggtatgttttctctccaggtgcagccagatctcacaaagtacaaaacgaatgcctttcttttcttgtttataatggtcactcactgtgtttggttactgtcaagaaatcaataaatgtgtttaacaagttacccagtaaaaaaaaaaaaaaaaa

SEQ ID NO: 8 - protein, 328 aa, Homo sapiens; identical to SEQ ID NO: 4

SEQ ID NO: 9 - protein, 328 aa, Homo sapiens; identical to SEQ ID NO: 4

SEQ ID NO: 10 - DNA, 1870 bp, Homo sapiens
  source 1..1870: mol_type = other DNA
atcccaccccaagccaacccgaggagtgtttgccgctatgagatcacctgctcccccagcccaacaacaaaattaaataacaccacgggagaggagagagggagggggaggagggaggaagggacaagagagaagagagactgaaacagggagaagaggcaggagaggaggaggtggggagagcacgaagctggaggccgacactgagggagggcgggaggaggtgaagaaggagagaggggagaagaggcaggagctggaaaggagagagggaggaggaggaggagatgcgggatggagacctggagttaggtggcttgggagagcttaatgaaaagagaacggagaggaggtgtgggttaggaaccaagaggtagccctgggggcagcagaaggctgagaggagtaggaagatcaggagctagagggagactggagggttccgggaaaagagcagaggaaagaggaaagacacagagagacgggagagagaagaagagtgggtttgaagggcggatctcagtccctggctgctttggcatttggggaactgggactccctgtggggaggagaggaaagctggaagtcctggagggacagggtcccagaaggaggggacagaggagctgagagaggggggcagggcgttgggcaggggtccctcggaggcctcctggggatgggggctgcagctcgtctgagcgcccctcgagcgctggtactctgggctgcactgggggcagcagctcacatcggaccagcacctgaccccgaggactggtggagctacaaggataatctccagggaaacttcgtgccagggcctcctttctggggcctggtgaatgcagcgtggagtctgtgtgctgtggggaagcggcagagccccgtggatgtggagctgaagagggttctttatgacccctttctgcccccattaaggctcagcactggaggagagaagctccggggaaccttgtacaacaccggccgacatgtctccttcctgcctgcaccccgacctgtggtcaatgtgtctggaggtcccctcctttacagccaccgactcagtgaactgcggctgctgtttggagctcgcgacggagccggctcggaacatcagatcaaccaccagggcttctctgctgaggtgcagctcattcacttcaaccaggaactctacgggaatttcagcgctgcctcccgcggccccaatggcctggccattctcagcctctttgtcaacgttgccagtacctctaacccattcctcagtcgcctccttaaccgcgacaccatcactcgcatctcctacaagaatgatgcctactttcttcaagacctgagcctggagctcctgttccctgaatccttcggcttcatcacctatcagggctctctcagcaccccgccctgctccgagactgtcacctggatcctcattgaccgggccctcaatatcacctcccttcagatgcactccctgagactcctgagccagaatcctccatctcagatcttccagagcctcagcggtaacagccggcccctgcagcccttggcccacagggcactgaggggcaacagggacccccggcaccccgagaggcgctgccgaggccccaactaccgcctgcatgtggatggtgtcccccatggtcgctgagactccccttcgaggattgcacccgcccgtcctaagcctccccacaaggcgaggggagttacccctaaaacaaagctattaaagggacagaatacttcctgttttctcagtggtctgattctaggcgcggtggggaaacatttgggtattaaagaacagacttcttccggaaaccaaaaaaaaaaaaaaaaaaaaaaaaaaaa

SEQ ID NO: 11 - protein, 328 aa, Homo sapiens
MGAAARLSAPRALVLWAALGAAAHIGPAPDPEDWWSYKDNLQGNFVPGPPFWGLVNAAWSLCAVGKRQSPVDVELKRVLYDPFLPPLRLSTGGEKLRGTLYNTGRHVSFLPAPRPVVNVSGGPLLYSHRLSELRLLFGARDGAGSEHQINHQGFSAEVQLIHFNQELYGNFSAASRGPNGLAILSLFVNVASTSNPFLSRLLNRDTITRISYKNDAYFLQDLSLELLFPESFGFITYQGSLSTPPCSETVTWILIDRALNITSLQMHSLRLLSQNPPSQIFQSLSGNSRPLQPLAHRALRGNRDPRHPERRCRGPNYRLHVDGVPHGR

SEQ ID NO: 12 - DNA, 987 bp, Homo sapiens
  source 1..987: mol_type = other DNA
atggaaatagtctgggaggtgctttttcttcttcaagccaatttcatcgtctgcatatcagctcaacagaattcaccaaaaatccatgaaggctggtgggcatacaaggaggtggtccagggaagctttgttccagttccttctttctggggattggtgaactcagcttggaatctttgctctgtggggaaacggcagtcgccagtcaacatagagaccagtcacatgatcttcgacccctttctgacacctcttcgcatcaacacggggggcaggaaggtcagtgggaccatgtacaacactggaagacacgtatcccttcgcctggacaaggagcacttggtcaacatatctggagggcccatgacatacagccaccggctggaggagatccgactacactttgggagtgaggacagccaagggtcggagcacctcctcaatggacaggccttctctggggaggtgcagctcatccactataaccatgagctatatacgaatgtcacagaagctgcaaagagtccaaatggattggtggtagtttctatatttataaaagtttctgattcatcaaacccatttcttaatcgaatgctcaacagagatactatcacaagaataacatataaaaatgatgcatatttactacaggggcttaatatagaggaactatatccagagacctctagtttcatcacttacgatgggtcgatgactatcccaccctgctatgagacagcaagttggatcataatgaacaaacctgtctatataaccaggatgcagatgcattccttgcgcctgctcagccagaaccagccatctcagatctttctgagcatgagtgacaacttcaggcctgtccagccactcaacaaccgctgcatccgcaccaatatcaacttcagtttacaggggaaggactgtccaaacaaccgagcccagaagcttcagtatagagtaaatgaatggctcctcaagtag

SEQ ID NO: 13 - DNA, 1697 bp, Homo sapiens
  source 1..1697: mol_type = other DNA
aacgcacgcctgcttgcactcacactgcggttcacacccggaggcgctctcgcactcacactgccgctcacgcgtgctcacactcccccacgcgcgctccgctccggctccagccccgcgcccagcgaaggcgcaggcactgctgccgagagcgccgaggggccccgcggccttcccatggcggacctgagcttcatcgaagataccgtcgccttccccgagaaggaagaggatgaggaggaagaagaggagggtgtggagtggggctacgaggaaggtgttgagtggggtctggtgtttcctgatgctaatggggaataccagtctcctattaacctaaactcaagagaggctaggtatgacccctcgctgttggatgtccgcctctccccaaattatgtggtgtgccgagactgtgaagtcaccaatgatggacataccattcaggttatcctgaagtcaaaatcagttctttcgggaggaccattgcctcaagggcatgaatttgaactgtacgaagtgagatttcactggggaagagaaaaccagcgtggttctgagcacacggttaatttcaaagcttttcccatggaggtaagaataacaaatcatcttgtaaaaatcttgttttctgaataaagtattcagcgatttactgaaaatgatttagtttaaaagtagatgcatagctcttgaattttatcagtttatactaaaatttaaaaaatgcttaatgcaatggaagcctagggcagcacatgaaacctcctgtctactctcgtggcttggcgtgtgcgcatgagcacatggccagaaaggcaatctacagtattaaatttcaccctagtgttactattcttgtaaaaattctgcctctgcaaattcagtaggtcatttttgtggatgctttggataggtgacgagctgaagacaagcaaccgttggagaaacctcaacagtaatgaaaagtgtaggtttgctagtttaaaattggtgggttggttttattcaccccaagccacttggggagggagggagaaagagagattttttgagagtgattcttttgtccaaagaattccctccccgacttacgttcttagttaacttctgctgtttctttgatacgcagttgaaatcttatgtcttctgtgggacttttctctagttttttccctaggcagaattcatctctctctctctgctccctgtatgcttgatagggcactgccatggcgctggccccatgggttgtaccatttagtctccacagcgtctttcttgctgcctgtgagctcctgaggaccgaagctccctctcaccccctttttactcccatcatttgcccactgccaggcacagtggagatagacagttcacactggtgaaagtgaggggatgttggattcagtccacgtcttgatgttatttctagaaggaacctcagtttaccccagaaaatagccctttggtgtcatgtaagagtatgttctgggggctgctggtcttccagtctttttttttgacattcacaactgtgcatgtgcttaatattaaatataaaattgctcatgaccagatgcaatatccagtaccacttcagtggctggaaatcatggctgtataattctatgtcagtgatacatgtattttagagtattctgttgaagtgtttgacagcattactgtaattataa

SEQ ID NO: 14 - DNA, 617 bp, Mus musculus
  source 1..617: mol_type = other DNA
aaaaaagaaacttaaattgaatgaggatgaagaagcagacctttagtgtgacccctgttaacttctctgtctgcagaacacatccagtcacctgcttcactagggacatcacattaccttgggaagccaaagtatacttcttgttgcccctgtttaagccaaccccagcccatactatacttgtggagggacagtgctgacactgacagcccagtgtacagcccacagctgtcaggctgcatctttgcttcacagatcctgtaggcccttcccttccccaaacattttttttttggggcaagatgtgaccaagccacctatttggatgggtgggctggcctccctctcctgacatctgccagtagactcccctccccttcctcccgccctgaaacctgatccagctgaaaagctgcttgctagaaaacttggcggctccccaactctggtggccgggaagtgccaggctgcagcctcacaaggtgcaggcgtgcagttggaaaagcagtctgcctgggacatgaggttggcagcgggtggcgtgcaggctggcagtctgggggactcatcctgagtggctgctctgaggccctcccacatctgggaagcttgccatg

SEQ ID NO: 15 - DNA, 611 bp, Homo sapiens
  misc_feature 104: note = n is a, c, g, or t
  misc_feature 352: note = n is a, c, g, or t
  misc_feature 356: note = n is a, c, g, or t
  misc_feature 370: note = n is a, c, g, or t
  misc_feature 375: note = n is a, c, g, or t
  source 1..611: mol_type = other DNA
gatgaggacaaaagggcagacctgtggagactccagttacccttcccgtaactacagaggtgcccagtcacctgcttggttatgtcaccctggggagtaagagnatggtcttcctgctcctgcctgactcaccctatcctgccccagcccagtgccacagaaacatacttgccaagggacatcaatgaaagcagcagcgtggtgcacctcccaggggtcaggctgcacccttgtctcatgggcttctgcagcccctgtccccacctaagttactccttggggaaacatggccaagccccctggctgaatgggtggactgagctcccacagcatgacatcagctagggcgggnctcncctctcagctgtcncctcncctttcctcccgcctgaaacatgatccagctgaaggactgattgcaggaaaacttggcagctccccaaccttggtggcccagggagtgtgaggctgcagcctcagaaggtgtgagcagtggccacgagaggcaggctggctgggacatgaggttggcagagggcaggcaagctggcccttggtgggcctcgccctgagcactcggaggcactcctatgcttggaaagctcgctatg

SEQ ID NO: 16 - DNA, 1345 bp, Homo sapiens
  source 1..1345: mol_type = other DNA
tcccgggagcggtcgtttgggagtgctccgtgggttaagcaaacgcaagctgtaagtgatgactgtggtcgagggcttccttgactgagaagaaactctgtccctcctagcaatggtcctttgccccgggcctgctgggatgagttcagtgctctccaagctggtcaggatcccagtcaataggacccaggctcttgctggtgtagtaaagacgcacacgggtctgcgtttgaatctcagttcctccacttgcagagcaaggtgctcacctttctggtgcttggggatccttgtgtgcaaatagccactctggtggactgttctaaagttaaatgaagaatatgtttattgatgttattccaaaggagacacttaaaaaatagtttgtgtgtgtgtgtgtgtgtgtgtgtgtgtgtgtgtgtgtgtgttctctctgctcttctctgtccttctgagggaagccagtagcttatcagggaaaagggggtgaaaggagggggaatctgcaggagtgatttgttgtacaggttgcacactagatgagagaagacacagtcttgtttagcaaaagccaggccttgccagcacctggttagtgaaagaccctggctacctgccagctccatctcccctttgctgcccgtatgctggccctctaggttgtactcccttcaccgtcccatgaattttcacttttgcatgtcaggccacccttctggaagtgctcctgctgcttccagtgtgaaaactgagctgtgcatgaaatcttcatagaacagcaccagggtccaggagctgggctgtctcattactggtttgtccccataggccacctaccccggaagtcgtttgatttcttctcactccctccacacctcgccccaaccacctatctaagcccatcatgtttgcccatacagcaaccctaggaggtggactgggccagccctccacccatagcttgtgtgtgcctggggcagggtgcgaggatcctcttggggtggcttttgcaggatgtagaggaaatgtcagttttcaccagatgcccacacttccttctttctttacctgtttcatactcaaaaaggagatgttgtgtgcagatgctgaggtttgaccttggctgaggaaaaccctgatgcagacccttgggcttagaaattgtgcacttggggtcatgctttcttgcaaccctcttgcttttagtatcataccctgagagccagcttccttgttgctccccagaggccctcgcttaggccccgtgggccacctgctccgaccttcttagccctcaacctgatttgaatctcagctgtcttgtgccacttgctcttccaagcca

SEQ ID NO: 17 - DNA, 1212 bp, Homo sapiens
  source 1..1212: mol_type = other DNA
ttttgattttaattagaagaaagactgccattagaattgttttctaatgaaatctttataaattctctgggcagtgcctgtatctttattagaaagacttcttatttttaacaattttatttggaagaagttctactcatttgcatcagttcatcgtctacataatttgtgtgtgtgtgtgtgtatgtgtgtgttacaactttgacttttgaaactagaatagaatcaggtaaaacatctaagatcagtgtgccagtaattggcctgaatcatcgggcaccatgatgggtttggccgccttcaacggtactcgtgtacattctatttttttcttcataatgttcagtactgtagtactaatcaccgagaaaattgcattgactcttttcgaccaccagggaaatattcagctcatggttctccccaaaaaaactaaaaagcagctaagcgctgggaacaaatctgacttattgcattttctcagtgggccaaagaaaggagggccgattgactgctttgactttttaaaggtcttctctttgttcacttataaagtgaggaaaacaaattctcggcactggcgtgagagttgagcgtcacaaaagaaagcaaaagaaaatattagtgccattattgtggcgaatttcatgtttcccagcgagccctttgattcctggtttgggctggcgctcgagctctccagccgggtatgacttcggccacaagatggcactgacctgcaaacaaagaaaagcacagtggcaccgactttttcaagcctcgggaaactgccctgccttccccggagtcgaggactgtggggattagggcttcctttcccctgcgcgggaggtctgtgtcgaataatgtgtggcttctgttggattgcttttctttccaaaattcctaggcaatgcttccccgaggtgtgcacctttgtgaggtgtttgtggggttgggggagcttcaggcgctactcgcgggacgacgtcacgtgatccgggatgaggtggagttcggctttaaggaggcgtctcttcctagcttcatcaatctttaggatctgagcaggagaaataccagcggatcttccccactctgctcccttccattcccacccttccttctttaataagcaggagcgaaaaagacaaattccaaagagggtaagttgcgagtttatgcctttccagagacttctgcgaaatctct

SEQ ID NO: 18 - DNA, 1148 bp, Homo sapiens
  source 1..1148: mol_type = other DNA
gagcaaaagtgtgaaaataaagtctttattaaactatggcaacttataaaaaataatatctttttaaaggtttactgggaattttcttataaactcttctatatctgacacctagacaaatgaaaccttctgaatagattttaagtattttaagaaagtagatagttctcaagaataataggaggaactgcagggaattcaaaggatattaaaagtctgacttgtacatttaaataaggtttctttgtttgaataattttttgctttatgaaaatacacatcattcttttcacaaaggatgacaatcacacaaaagaataaaatatatgtaattctttttcctcctgcaatgatacaatatcaactattgtctgttttgctgtgatcgtggaacagcagcatgattccatgttctcaaatattcccatggttttttcttaaagccagaatctggcacaatttgaattggaaagataaatacagctctgctcagctgacaaacacacaggcatgcaccaccctcccttccaaattctaacagcagagtttctcttcccttgagaaatctgaaatctaaaaattgcattcggactgccaaaaattctgctgaaaagagccttaggctattttccatggtgtaaggtaaaatcgttttggctttggtccaacacaacgcctcagttcctttaaaagattaactaaaggtctgtgaacttggaaaaaagaattccttggaattcgagttctatggatgtttgttattgctcgcatttgttatatgaagacacctcgcaaacaggagcgcaaaccttagagcgcagctgcctcccgggctcgggtcagcgctggaccgcgcggcagctggacggcctgagtggcgccccgcgggctgcgcccccggcctcgccagagacccgctaggttgagccctgacccgggcaccccgcgggctgctgttcccagctccggatgccagccgggctagcgccagagcgtgtccagccgggggcagcgctttgtccatcccccgccccatccccaggaggctccttcgcggatccccgacactctcctcccgggttcggcccgcgcctgctccacccgggtgagttgggaaccggagggacctgctcggcacgaaagaattc

SEQ ID NO: 19 - DNA, 1308 bp, Homo sapiens
  source 1..1308: mol_type = other DNA
aaatattctttaaaattatttaataatggaaatacagataccatttagacttcacattttagaatagttttgatgtatatgaaaacaacttttattggtagtgtttaatgttgtataatgagtctggaaaggatttaaaattctttatttggtaaattacttgtgtattactaggacagtggagttaaattgacaatttgaccaattctcaataggcaatagcattttcctacaggacttccaaaactgtagttaatgttgaagattataatcataatttgggcatacatctgtcatttagttgtgattttccaggcatattaaattttttctgtacttcacaaactaaacattaagaagcctatggagtagaaatgatatagatagctggtagatattcttcaagtttgaaattaaaagttcctcgagaaaaattttttcttattatatttttggacaaaagtactgagttggttagatataacttttactttcaacaaaaaatacaattcatttgtttataagacaacatggaagtggaaggtaatacaaccttctaaagtgaaattacagattggattgacactgactgcacaactaaataaattaatcctactataacacataaaattatcttccatatattatgaactgacatttgaatccagaactctttcacataaattaaattccatgttaacaaaatttctatttattatatgtaaatagttatcataaatatattttatatgaccccagcatttgttctttccttggtaaacagtgagaattattacatatgaagacagtgtataagggacactaaatcagctttaatcccattattaagaaacccatatagaattggattaagacaacaggacatcatgcccacaatgacaatgcaatacaccaacttgaaatgcctatttgaaataagattgtcattaaaatcacttaccacatggaaaaggtgtagtggcatccaactaacagcaaatactaatatcagtatggtcagtctgtagaaaacacttcgagatctcttttttattcttgtaacagaacgttttactctcaattcatgaacatctgaattttcttcaggttttatctcaaagcaagtggggacccctggtatgaacttactggatgaagagagctgacttcttacagagccaaagttttctggaagtattctggagtggttctcttgagaaggtctttctggagcaggtaacacacatgctgtcttcttgctatatcttcttctgtgttttttgatgaatgaataactccatttatggc

SEQ ID NO: 20 - DNA, 731 bp, Mus musculus
  source 1..731: mol_type = other DNA
cggagaacatttttgtttcagcatttcatctgaagccacggtttcacatcatcaagtctgcaaaaaaccgttcacaaaccacaccaaaacttctcggtaaagaactcctaaggccaaagagggagactgggtagattgtttttaatttgtttctttttgtcaaagggggacaaaacacgctttggtgagtgcgagtgtttattctgggacacaaacccagagtctggaagggagcattcaacgggtgctgctctgccacgcaggggcagcggtgggactcagcccatcctgctaaggacgggcagcctgagccaggcttgggagtctgtcatggctgccagacgaatcattatctaattgcagccttttctcttccttaggtttcagcaggtcccgagagagcatttaaaatcacatttactactttaccatctaatcacacataagcctctccctataccctccaccctccttccattcagagtgtactttctggagcaccatccagcaagcagggtggaactcgtgacgggaaatgggaacggcacccacgaaggcgtgattccttgtagatccttgagtgacggacgggtgaggtttccgtcaggcaagcccagccaccttcgtggaggagccccggacaagtgtaagtttcgcagagctggggtctccagcttacttctgctaatgctaccccaggcctttagacggagaacagatggcagatggag

SEQ ID NO: 21 - DNA, 820 bp, Rattus rattus
  source 1..820: mol_type = other DNA
ttgagaacatttttgtctcagcatttcatctgaagctatggttttacatcaccaagtctgcaaaaaacagttcgcaaacggaaccaaaactttttcgacaaagaacttttaaggcgaaaacgggagatagagtagattgtttttaatttgtttctttctggcaaagggggacaaaacacactttgacgagtgtgagtgtttattctgggacaaacccagagtatgaaagggagcattcggtgggtgccgctctgccatgcaggggcgcggtggggctcagcccatcctgctcaggacccagcctggccccaggcttgggagtctgtcatggctgccagaggaatcattatctaattgcagccttttctcttccttaggtttcagcgcgtcccaggagagcatttaaaatcacatttactactttaccatctaatcacacataagcctctccccataccctccaccctccttccattcagagtgtactttctggagcgccatccagcaagctgggtggaattcgtgacgggaaatggaaacggtacccacgaaggcgtgattcctcgtagctccctgagtgaggttcccatcaggcaagcccggccaccttcgtggagaagcgcggacaagtgtaagttttgcagagctggggtctctagcttgcttctgctaatgctaccccaggcctttagacagagaacagatggcagatggagtttcttattgccatgcgcaaacgctgagcccacctcatgatcccggaccccatggttttcagtagacaacctgggctaagaagagatctccgaccttatagagc

SEQ ID NO: 22 - DNA, 834 bp, Homo sapiens
  source 1..834: mol_type = other DNA
ttttttttttttttgtaatttcatttcaaactattattttataagacctggcctattactgagtatgcaggcagaatatgaaaattactccaaaacttttttaaatgaaattttcaagatgcaaaaagtgaaactttaaaatttcagtggaagaaggggaacaaaaacattttaataaatgagagtgtttattccagaatgggaatatagagacaaggaaggtaccatgtgaatgggtgcacctcgctctctggggtcaatgataggaaacagcctgtcccacagtcaaggcagccttgcccaggctatgagtctattgtggatgctggggcattgttatctaagtgcagcctctttgcttcctcaggtttcagcatttcccatgagatcatttaaaatcacatttgctattttaccatctaatcacacataagcctctccccacactccccccgccctgtttccatccaaggagtgcactttctggagcaccagcaaccagggtggaactcgtgacgggaaatgggaatggcacccaagaaagcatgatttctgtagtttcgtgaatgatagcaaggctcccatcagacaagctgagccactgtcactgaggaggacaaacgagtgcaagtctttgcaaagcttggcatctcagacttgcctctcatttcttgcttcacacactagcctcttggctagagaacagacatcagatggagtttcttctggctatgcctgaatgttaagctgaacgtatgttccaggagctcgtggtctccagtagaggcaatctgggatagaagagaagatatttcttacgtagaagacaagcaa

SEQ ID NO: 23 - DNA, 1452 bp, Homo sapiens
  source 1..1452: mol_type = other DNA
gctgaggtgggagaattgcttgagcccaggagacagaggttgcagtgagcagagattgcaccactgcactctagtccgggtgacagtgataccctgtctcaaaaaaaaaaaaagaaaaagaaaaagaaaaaaacaaaaaagacaagaactgagaagggccccctgactttagccacaaggcaatctcttgtatccctgcaaggaagcattttagtgagataggagggcaaaatccaagcttcaaagactgaaaagtgactggaaacagtgagtagaagtaagtcctttattaaaagccaatagagaacaggagagaggagggaagacctggatgggctctgcatcaagggaaagtttttaaagttggaggaaagttaagcatctttaaatgctgataggaaagagctggcagacaaggagagagaaaaaaatgaggtagatatgagataagatcctaagcagagggaaaggaatggtccttagaatgtaggaagatacctctcatgttgtaagagaagggaaggaggaaagaaggctgcagacacaggtgagttggaggtttgatggtatgaagttggcagagttctgtttttctccatgaactaggaaatatggtcattggcttactgcaagggaagacagggaatagtgagaaaagagtgtatttgaaacctcagactagaagaaactgctgactagggataccatatgattcccaggcaatactgaggttccccataacccctcagcagttcaggggaactgttctagattgacttagccttgggatttaggataagggtgagggagtggcaaaattgtggaccacaggtctaatctggttatggggaaaagtgttcagccacagactgaacctggaggtcccagagaaattggaggtcccaatgaaattctccaataatctggagttctccagtaacttggaggtccaaatgaaattcaaacaggtggagaaatgttgacaatgtcaataaataaaagattgtgataagaaagtagcatgagacttaagataaagtagactataaaccaaagtctttgatgaatatgggagagtgcctggaactcagtggtcctggaaatgaggagagagggcagcatggctaaatctggtggtggtggtaggatggtacaaagtggtgaagaagcaatgacatagaagcacatggggagcaaagacataaccctgaggcacctggaggagggttgaataaacagcctcccctgaatgccttgcaggggaatgggttcctgggaggagccaagtgtgagttcagggaggggccagatatgagggtgggagaagggctgttctgacaatcaagatgggacagatgagagggacagggccagtgggcaagctgtcacctctctgtggttatgtccactcttataagagtataaatacttcctgaagaagaatgagaagat

SEQ ID NO: 24 - DNA, 1534 bp, Homo sapiens
  source 1..1534: mol_type = other DNA
accaccaatcagggaagagaaggcgatttgtccaggatatggcactgagaattatcacaagtgagctagttcaccttcattgcacagtctcagtcactgaagtttccttccttcaatagcacatcttacaagtcaagaatgtaaggtccaaagccccaggacaaatctcagaagcaaagaaagcagcagggaaaagtagaccctgggatttgatttccttccggttatctctaagcatcatttccatgatagaaggtgtggaagcaaataataaaagtgcccgtcactagtgtttatcctgcaaagtggtctgcccttttgagaggcaccctgccctatggccatcctttgatttcttcccttggtggaaatttcctgtttctctttgagaaagataattcaaccctgtttccattgttcttcctcccaggctgctgtaggaaagcgcacgcacacactccacacaaaatgaatttttaaaaaatttattttcacagtcgctcctaccagctctgaaattcagaacccatatgactgatggcatattcagataatcgggtcccaggtctggaaaagcagccttttccccacgtttctttccccacctaggacctcctctgattcttcactgcatcttcgaaagaaaatgtattatttgcttgcctggaagacgctgcaattcaattgattttatatatacatatatataaagaaaacagaaaacatagcctagataccggtcttgagcgtcaccgccccactcgcggttgtgagcaaagccctacggaagaaaccaattcccagcctagactcttcagagcccaaggtcggggaggcgctggcctggcggtgttgtctggctccccagccactgccccagactcagggctttgccattggtccccacctcctctgctccggagtttttctccagctccccaccaagccacacaaagtgacttctcggaaacattagccgattctgctgagcaggaagggaggaaagggatgatgggggcgggggtgagataagggaagggctcttctggctgctggacacacacacacacacactcaaacacacacacgccccacccaatgggtggccgtggatggcaggtcgtgcaaccccctcctccgccttctattagcgcatggtgcagaggctacagcgtcgccaccaccgcgcccctagctgggtccccgccctgcgccgcccgcaggagtggagagagggagggagggagggagcaaggggtggggacccgggcgcgctgggaggagtggaggaggcaaagcggcgcagctgccctcggggaggcggggctgctacctccacgggcgcgccctggcaggaggggcgcagtctgcttgcaggcggtcgccagcgctccagcggcggctgtcggctttccaattccgccagctcggctgaggctgggctagcctgggtgccagtggctgctagcggcaggcgtcccctgagcaacaggagcccagagaaaaagaagcagccctgagagagcgccgggga

SEQ ID NO: 25 - DNA, 138 bp, Homo sapiens
  source 1..138: mol_type = other DNA
cctaacaggggagggggcagaggggggggcgtcagagagtaggaagcgggtggagaagaggggcaaggcggggccgggcgggggccgctggctccgccctttcctggcggctgggtctttaacaccgcccagcgcaca

SEQ ID NO: 26 - DNA, 1469 bp, Homo sapiens
  source 1..1469: mol_type = other DNA
gacatctgcggggtaacttctggcagcctcgacccgggagaacgtggacagcccttggcctctgggagcccctctcagcctcctaggaggcctccttgtcctctttggagaccccttagcgccaggctccgtcaccgcctagtcccttggttctgaccaagatcccgaggaagaaggcgatcactgtgtagggctctgcctccgtctggtcaccttcttgagctcaggctgctcggtcggtctgtgtgtccctgtctcggggacttggggggccatccgtgctggggccttggcggagagaaccgtgagcctctaggaggtcgtccttcccccgctccctggggccttgcctgtctccccgccaagagacacccctcttcccttcgctctccccagcttgagacggatgggttagtgcagccacggaggcttgcgcggtgggaggggttgggaccagccttctgctgccctgggtgctggggatcccggggctttccaggtgctcggcctccaaggtgcgcggtcctcagctccacccgcgggcggctcctgcgtccgaggagctaagagaagatctattaatttcttcacgaataaatcgatgctcttgtcagggaggcgatcgatgtcagccctgccctgccttgccctatcctgccccggggccggcgctggctggccggggtcagggactgaagctgagacctgaggcgttgctcactgggggctgcagatcgcacccccaggcacccagcgcgggcggggagctcgcgcctttgcgcgcgggcttctcgcgccaccctgtggcttctcttggaggcgcggtcttggctctccggactcccttcggccggattaggcgaccccttccctttctctgccccgtctgtgtcttcctccccaggttctgcgattgatcctttggtagtccttttcgttttcttcctagagttcggagaatgttctacctaacttactccaagtgacatgctcactcccctaggcacgcgcgccgcgaggatggagcgctgagcctggggctggctaggatgacctggacagcaacctttcctcaacgcagtcatcttccctcctccccaaatgtaaaaatgcagctgctttaagctgagagaaataacgtatcagcttcccacctccggcctcagcagacacctccgaggcgttctgctgcggcccctcagcgtctgccggagctgaggcggatcctcggggagaaggctgacgctgggggcccctaacaggggagggggcagaggggggggcgtcagagagtaggaagcgggtggagaagaggggcaaggcggggccgggcgggggccgctggctccgccctttcctggcggctgggtctttaacaccgcccagcgcacatgtcgggggaggcctggcagctgcagctgggagcgcacagacggctgccccgcctgagcgaggcgggcgccgccgcg

SEQ ID NO: 27 - DNA, 1306 bp, Homo sapiens
  source 1..1306: mol_type = other DNA
tagatggcgcatgctctccaaacttggctttgtccatcaaggttcaagaaaacaatggtcagacatgttcctcttaacaaacagtatgtccccaaacagcaaaaatgcatacagtcctttctgggtgaatttttaaatcttacataaatccatcaaccccatcctttttcctttgcctcttgggagaaattaatctagctttacattaattatgcatgttatcagatttcaagctccttgagagcaggtattttaattctataaagcctctacgtggccttggacatgggaggtgcttaattacccaagatgctccttgaatacagatggtacacgacctacacagacttagatctttaccacttcccccctctccccaccctgacttgctcaatcctgaaggaactggagacgtctaagtgtctgaggttcacgcttccacacagaagcttgggtctgtgtgggagggaaaaaggaagccatctgtccgcaggccagaccaggccacaccctgctagcacccagaaccctttgtcccaggcccagccctgccattttactttccttgcatctggaaagcacagggaatatagtagtgacaaaagaaggaagggttgtttgagtttaagaatagtttactctaaaaaaaaaaaaaaaaaaaaaaaaaggacaaaagccaaagagaaggtcaaagttgactgtggagaaggccttgcaagcagggaacttgggaagaattggaatgagagtgagagaaggcaactgagtttggaaatattttttctgactagcttttctttccaaatgccactgaacttagattggtttaggaagggttgtagtacatcaaagtggctagaagcacaggtttggggatcagataaggatttcattctagagtgtgatcttgtacaagttattcagcctttgcaaacctcagattcacacaatgtaagatgaagaaactcaccttctgaaaattagagataacatatgcaaagtgaatcaatacagggcttaacatatttatcacccctttggtaaataaccatgacgattaccagagctcttaagggcaatggcaggtgggaagcagaactcatgggtggtaatccccaggccagccaggctcaccatgtgcacttggacaagtccttgcccccatcattgtgaaatggtgcagggatgcaccatgagggtgtggcaggatggctgacaacagactgggaagcagctcggcagaaaaactggattgatgcccactatggcaagagatatcatctcccctcttgttctgtgatgtttc

SEQ ID NO: 28 - DNA, 1554 bp, Homo sapiens
  source 1..1554: mol_type = other DNA
accgcccgagagactcggaggcaggcttgggacacgtttgagtgaacacctcaggatactcttctggccagtatctgttttttagtgtctgtgattcagagtgggcacatgttgggagacagtaatgggtttgggtgtgtgtaaatgagtgtgaccggaagcgagtgtgagcttgatctaggcagggaccacacagcactgtcacacctgcctgctctttagtagaggactgaagtgcgggggtgggggtacggggccggaatagaatgtctctgggacatcttggcaaacagcagccggaagcaaaggggcagctgtgcaaacggctcaggcaggtgatggatggcagggtaggaagggggaggtccagaggtctggatggaggcttccgcatctgtaccttgcaactcacccctcaggcccagcaggtcatcggccccctcctcacacatgtaatggatctgaagagtaccccgggacagtccggggagatggagattcggaaagtatccatggagatcttacagaatcccctgtgcggaccaggaaactcttgtagatccctgcctatctgaggcccaggcgctgggctgtttctcacaatattccttcaagatgagattgtggtccccatttcaaagatgagtacactgagcctctgtgaagttacttgcccatgatcacacaaccaggaattgggccaactgtaattgaactcctgtctaacaaagttcttgctcccagctccgtctcttgtttcccacgagccctggccctctgtgggtaataccagctactggagtcagatttcttgggcccagaacccacccttaggggcattaacctttaaaatctcacttgggcaggggtctgggatcagagttggaagagtccctacaatcctggaccctttccgccaaatcgtgaaaccaggggtggagtggggcgagggttcaaaaccaggccggactgagaggtgaaattcaccatgacgtcaaactgccctcaaattcccgctcacttaagggcgttacttgttggtgcccccaccatcccccaccatttccatcaatgacctcaatgcaaatacaagtgggacggtcctgctggatcctccaggttctggaagcatgagggtgacgcaacccaggggcaaaggacccctccgcccattggttgctgtgcactggcggaactttcccgacccacagcggcgggaataagagcagtcgctggcgctgggaggcatcagagacactgcccagcccaagtgtcgccgccgcttccacagggctctggctggacgccgccgccgccgctgccaccgcctctgatccaagccacctcccgccaggtgagccccgagatcctggctcaggtatatgtctctccctccctctccctccattcgtcattttctcactccctttcctcctctccctctctctccgttagtctcttcatcagatagtctctgttagtccgcgatttataccaggctcgtgccctaggttggatcggacagtctcaatcccccggctcgctcttcctgct

SEQ ID NO: 29 - DNA, 1297 bp, Homo sapiens
  source 1..1297: mol_type = other DNA
ggctggggaaaacagcattcactttgcaagcttcttgggatttttacttaactagagtctcttcttcccaggatttaaaacatgatggaaactttagctcatgaatcaaaacaaccctccagagctaaaagccagctgtatttgcataacaatttagcagatccaaacagcagggcaaggtcgggtgaaataagttgccaaggtcatggtcatgaagtagtattagactcagaaaggctgatccccagtgcttgctccaccccatggatctctcctaccctccttctaaacgatactgtgggataaaataaaattaatctactgtatatgtgcaaaccacaggcctgcccttaactctttccttaccttctagtttcagattattcaaatcatggaggaaaagattagatcacaacacgttgacttcactgtattaccatacaaatgaaataacttagtacaaactgtgatctggggactcttgatctaaactgggaactgctgttgactgcattttaaactctaaaagtattttgaaactctttaatttcttgaactgaaaaaattgctttgaattcactttgttttaattctgagaacctaaaaacagggattctttaaaaaaaaaatgcaaaggctcacatgccagaaagaaagaagctgaggagataaaaatgtgtaaataattcttactttaatacccttagctagaaaaaccttaaaagcgacacatccagaagctcgttaagtcacagcctctttgaacctatttcagtgaaccaccgaatttcagatccctcaggtgcgactctgaattcagaattctcaccggctcatagtcctattttccttcttaggttttagggaattttgcaaactatgacgcccagcctttgaggggagaggactttccaggggcgcgggatgtgccactcgggaatctcaccaacagtgggcgtttagcgcagccaagcgacaggcaggcgccagggctcagcaacagggaggcgccggctgaggcggggagaactttggcgctcggagcagagccaccctttgctggccagtcgcgttgctcctccgaggaagcaagcggcggtggcgactcggtggaaaaataacgaaagaaaggcagagaggaagtagcgagagaagagagaaaatgaagtcggcgctgggggagcctgcaggagggtggccaacagtggaggaaggtggatttggcttcttttccgcaccccgggcgtgaaagccctctccaacgcgaccccaggaaataagtgggtctcgcctgggc

SEQ ID NO: 30 - protein, 139 aa, Homo sapiens
MADLSFIEDTVAFPEKEEDEEEEEEGVEWGYEEGVEWGLVFPDANGEYQSPINLNSREARYDPSLLDVRLSPNYVVCRDCEVTNDGHTIQVILKSKSVLSGGPLPQGHEFELYEVRFHWGRENQRGSEHTVNFKAFPME

SEQ ID NO: 31 - protein, 153 aa, Homo sapiens
MADLSFIEDTVAFPEKEEDEEEEEEGVEWGYEEGVEWGLVFPDANGEYQSPINLNSREARYDPSLLDVRLSPNYVVCRDCEVTNDGHTIQVILKSKSVLSGGPLPQGHEFELYEVRFHWGRENQRGSEHTVNFKAFPMEVRITNHLVKILFSE

SEQ ID NO: 32 - DNA, 1710 bp, Homo sapiens
  source 1..1710: mol_type = other DNA
aacgcacgcctgcttgcactcacactgcggttcacacccgcaggcgctctcgcacacacactgccgctccgcgggctcacactcccccacgcgcgctccgctccggctccagccccgcgcccagcgaaggcgcaggcactgctgccgagagcgccgaggggccccgcggccttcccatggcggacctgagctgcatcgaagataccgtcgccttccccgagaaggaagaggatgaggaggaagaagaggagggtgtggagtggggctacgaggaaggtgttgagtggggtctggtgtttcctgatgctaatggggaataccagtctcctattaacctaaactcaagagaggctaggtatgacccctcgctgttggatgtccgcctctccccaaattatgtggtgtgccgagactgtgaagtcaccaatgatggacataccattcaggttatcctgaagtcaaaatcagttctttcgggaggaccattgcctcaagggcatgattttgaactgtacgaagtgagatttcactggggaagagaaaaccagcgtggttctgagcacacggttaatttcaaagcttttcccatggaggtaagaataacaaatcatcttgtaaaaatcttgttttctgaataaagtattcagcgatttactgaaaatgatttagtttaaaagtagatgcatagctcttgaattttatcagtttatactaaaatttaaaaaatgcttaatgcaatggaagcctagggcagcacatgaaacctcctgtctactctcgtggcttggcgtgtgcgcatgagcacatggccagaaaggcaatctacagtattaaatttcaccctagtgttactattcttgtaaaaattctgcctctgcaaattcagtaggtcatttttgtggatgctttggataggtgacgagctgaagacaagcaaccgttggagaaacctcaacagtaatgaaaagtgtaggtttgctagtttaaaattggtgggttggttttattcaccccaagccacttggggagggagggagaaagagagattttttgagagtgattcttttgtccaaagaattccctccccgacttacgttcttagttaacttctgctgtttctttgatacgcagttgaaatcttatgtcttctgtgggacttttctctagttttttccctaggcagaattcatctctctctctctgctccctgtatgcttgatagggcactgccatggcgctggccccatgggttgtaccatttagtctccacagcgtctttcttgctgcctgtgagctcctgaggaccgaagctccctctcaccccctttttactcccatcatttgcccactgccaggcacagtggagatagacagttcacactggtgaaagtgaggggatgttggattcagtccacgtcttgatgttatttctagaaggaacctcagtttaccccagaaaatagccctttggtgtcatgtaagagtatgttctgggggctgctggtcttccagtcttttttttttgacattcacaactgtgcatgtgcttaatattaaatataaaattgctcatgaccagatgcaatatccagtaccacttcagtggctggaaatcatggctgtataattctatgtcagtgatacatgtattttagagtattctgttgaagtgtttgacagcattactgtaattataaaaaaaaaaaaaaa

SEQ ID NO: 33 - DNA, 1710 bp, Homo sapiens
  source 1..1710: mol_type = other DNA
aacgcacgcctgcttgcactcacactgcggttcacacccgcaggcgctctcgcacacacactgccgctccgcgggctcacactcccccacgcgcgctccgctccggctccagccccgcgcccagcgaaggcgcaggcactgctgccgagagcgccgaggggccccgcggccttcccatggcggacctgagctgcatcgaagataccgtcgccttccccgagaaggaagaggatgaggaggaagaagaggagggtgtggagtggggctacgaggaaggtgttgagtggggtctggtgtttcctgatgctaatggggaataccagtctcctattaacctaaactcaagagaggctaggtatgacccctcgctgttggatgtccgcctctccccaaattatgtggtgtgccgagactgtgaagtcaccaatgatggacataccattcaggttatcctgaagtcaaaatcagttctttcgggaggaccattgcctcaagggcatgattttgaactgtacgaagtgagatttcactggggaagagaaaaccagcgtggttctgagcacacggttaatttcaaagcttttcccatggaggtaagaataacaaatcatcttgtaaaaatcttgttttctgaataaagtattcagcgatttactgaaaatgatttagtttaaaagtagatgcatagctcttgaattttatcagtttatactaaaatttaaaaaatgcttaatgcaatggaagcctagggcagcacatgaaacctcctgtctactctcgtggcttggcgtgtgcgcatgagcacatggccagaaaggcaatctacagtattaaatttcaccctagtgttactattcttgtaaaaattctgcctctgcaaattcagtaggtcatttttgtggatgctttggataggtgacgagctgaagacaagcaaccgttggagaaacctcaacagtaatgaaaagtgtaggtttgctagtttaaaattggtgggttggttttattcaccccaagccacttggggagggagggagaaagagagattttttgagagtgattcttttgtccaaagaattccctccccgacttacgttcttagttaacttctgctgtttctttgatacgcagttgaaatcttatgtcttctgtgggacttttctctagttttttccctaggcagaattcatctctctctctctgctccctgtatgcttgatagggcactgccatggcgctggccccatgggttgtaccatttagtctccacagcgtctttcttgctgcctgtgagctcctgaggaccgaagctccctctcaccccctttttactcccatcatttgcccactgccaggcacagtggagatagacagttcacactggtgaaagtgaggggatgttggattcagtccaggtcttgatgttatttctagaaggaacctcagtttaccccagaaaatagccctttggtgtcatgtaagagtatgttctgggggctgctggtcttccagtcttttttttttgacattcacaactgtgcatgtgcttaatattaaatataaaattgctcatgaccagatgcaatatccagtaccacttcagtggctggaaatcatggctgtataattctatgtcagtgatacatgtattttagagtattctgttgaagtgtttgacagcattactgtaattataaaaaaaaaaaaaaa

SEQ ID NO: 34 - DNA, 1825 bp, Homo sapiens
  source 1..1825: mol_type = other DNA
cctcgctctgctggggcctccggacgcgcttcccacgcgggtctctggaacactcggtccgaacgcacgcctgcttgcactcacactgcggttcacacccggaggcgctctcgcactcacactgccgctcacgcgtgctcacactcccccacgcgcgctccgctccggctccagccccgcgcccagcgaaggcgcaggcactgctgccgagagcgccgaggggccccgcggccttcccatggcggacctgagcttcatcgaagataccgtcgccttccccgagaaggaagaggatgaggaggaagaagaggagggtgtggagtggggctacgaggaaggtgttgagtggggtctggtgtttcctgatgctaatggggaataccagtctcctattaacctaaactcaagagaggctaggtatgacccctcgctgttggatgtccgcctctccccaaattatgtggtgtgccgagactgtgaagtcaccaatgatggacataccattcaggttatcctgaagtcaaaatcagttctttcgggaggaccattgcctcaagggcatgaatttgaactgtacgaagtgagatttcactggggaagagaaaaccagcgtggttctgagcacacggttaatttcaaagcttttcccatggagctccatctgatccactggaactccactctgtttggcagcattgatgaggctgtggggaagccgcacggaatcgccatcattgctctgtttgttcagataggaaaggaacatgttggcttgaaggctgtgactgaaatcctccaagatattcagtataaggggaagtccaaaacaataccttgctttaatcctaacactttattaccagaccctctgctgcgggattactgggtgtatgaaggctctctcaccatcccaccttgcagtgaaggtgtcacctggatattattccgataccctttaactatatcccagctacagatagaagaatttcgaaggctgaggacacatgttaagggggcagaacttgtggaaggctgtgatgggattttgggagacaactttcggcccactcagcctcttagtgacagagtcattagagctgcatttcagtagccaaagaggacaggaacaagtctgtcttcatgagggtatgtccagattaagtaaggaaaatgaggtgcacagctacttaaacctagcgtaccaaaggtacacttattgtgatcctggtaatttagtaataaaagcgtatgtacttcatcttgataaaatttatgacacattcttgagctctgttaattacaggtcaagtaggtattttaattttatgcagtattgaagaaaaactatgattatattttcctatcagagtagcctttaattccaagttttggtggctttcataagaacagacaccctcaggcccagaaaaagtgacatgcatgcatacctctttagtaacttaccatgttgtccccgtgaagcagcaccagtcatgtcccagtgaccaaggagcacataggaggggacttggggagcgtgtgagcctcagtgtggacccttctcgtctcctgcagaattgatcctcttgtttctcaggggagggagcatatctggcatgcattgaaatacttcttctatggaagtgccttttatatgataaattgagaagcacttattaattatctgaatatgatacactattgattgaaacataatctatgtattttaatatatagaattcaatgtatgtattccttactcttaaggaactaacaaaataaaatatcacaaacacggaagagataaaatataaaacaaactata

SEQ ID NO: 35 - DNA, 510 bp, Homo sapiens
  source 1..510: mol_type = other DNA
gaaagataactgaatcaatgtgaagtctgtgagtccctccagttgtcttcctcctcatcaggactaaaactggacttaagaggcagtgatccactaagcactgcttctgttcagaaataagaaggaaggaattcatacagagaactcgtattaagccagtagtttgtgtctacttgtgatcctcttgtgctccatctgatccactggaactccactctgtttggcagcattgatgaggctgtggggaagccgcacggaatcgccatcattgctctgtttgttcagataggaaaggaacatgttggcttgaaggctgtgactgaaatcctccaagatattcagtataaggggaagtccaaaacaataccttgctttaatcctaacactttattaccagaccctctgctgcgggattactgggtgtatgaaggctctctcaccatcccaccttgcagtgaaggtgtcacctggatattattccgataccctttaactatatcccagctacag